SEQUENCE LISTING (49 sequences)

SEQ ID NO: 1 - protein, 495 aa, Artificial Sequence
  Description of Artificial Sequence Synthetic polypeptide
MRCVGIGNRDFVEGLSGATWVDVVLEHGSCVTTMAKNKPTLDIELLKTEVTNPAVLRKLCIEAKISNTTTDSRCPREGEATLVEEQDANFVCRRTFVDRGRGNGCGRFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGVSYVMCTGSFKLEKEVAETQHGTVLVQVKYEGTDAPCKIPISTQDEKGVTQNGRLITANPIVTDKEKPVNIETEPPFGESYIVIGAGEKALKLSWFKKGSSIGKMFEATARGARRMAILGDTAWDFGSIGGVFTSVGKLVHQVFGTAYGVLFSGVSWTMKIGIGILLTWLGLNSRSTSLSMTCIAVGMVTLYLGVMVQA

SEQ ID NO: 2 - protein, 495 aa, Artificial Sequence
  Description of Artificial Sequence Synthetic polypeptide
MRCIGISNRDFVEGVSGGSWVDIVLEHGSCVTTMAKNKPTLDFELIKTEAKQPATLRKYCIEAKLTNTTTASRCPREGEPSLNEEQDKRFVCKHSMVDRGRGNGCGRFGKGGIVTCAMFTCKKNMEGKIVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQA

SEQ ID NO: 3 - protein, 493 aa, Artificial Sequence
  Description of Artificial Sequence Synthetic polypeptide
MRCVGVGNRDFVEGLSGATWVDVVLEHGGCVTTMAKNKPTLDIELQKTEATQLATLRKLCIEGKITNITTDSRCPREGEAVLPEEQDQNYVCKHTYVDRGRGNGCGRFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQA

SEQ ID NO: 4 - protein, 495 aa, Artificial Sequence
  Description of Artificial Sequence Synthetic polypeptide
MRCVGVGNRDFVEGVSGGAWVDLVLEHGGCVTTMAQGKPTLDFELTKTTAKEVALLRTYCIEASISNITTATRCPREGEPYLKEEQDQQYICRRDVVDRGRGNGCGRFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 5 - protein, 504 aa, Artificial Sequence
  Description of Artificial Sequence Synthetic polypeptide
IRCIGVSNRDFVEGMSGGTWVDVVLEHGGCVTVMAQDKPTVDIELVTTTVSNMAEVRSYCYEASISDMASDSRCPREGEAYLDKQSDTQYVCKRTLVDRGRGNGCGRFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 6 - protein, 495 aa, Dengue virus
MRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGVSYVMCTGSFKLEKEVAETQHGTVLVQVKYEGTDAPCKIPISTQDEKGVTQNGRLITANPIVTDKEKPVNIETEPPFGESYIVIGAGEKALKLSWFKKGSSIGKMFEATARGARRMAILGDTAWDFGSIGGVFTSVGKLVHQVFGTAYGVLFSGVSWTMKIGIGILLTWLGLNSRSTSLSMTCIAVGMVTLYLGVMVQA

SEQ ID NO: 7 - protein, 495 aa, Dengue virus
MRCIGISNRDFVEGVSGGSWVDIVLEHGSCVTTMAKNKPTLDFELIKTEAKQPATLRKYCIEAKLTNTTTASRCPTQGEPSLNEEQDKRFVCKHSMVDRGWGNGCGLFGKGGIVTCAMFTCKKNMEGKIVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQA

SEQ ID NO: 8 - protein, 493 aa, Dengue virus
MRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQA

SEQ ID NO: 9 - protein, 495 aa, Dengue virus
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 10 - protein, 504 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 11 - protein, 619 aa, Dengue virus
SGVLWDVPSPPETQKAELEEGVYRIKQQGIFGKTQVGVGVQKEGVFHTMWHVTRGAVLTYNGKRLEPNWASVKKDLISYGGGWRLSAQWQKGEEVQVIAVEPGKNPKNFQTMPGTFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFMGQPLNNDEDHAHWTEAKMLLDNINTPEGIIPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRK

SEQ ID NO: 12 - protein, 619 aa, Dengue virus
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGTFQTTTGEIGAIALDFKPGTSGSPIINKEGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGILPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRK

SEQ ID NO: 13 - protein, 619 aa, Dengue virus
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGRNPKNFQTMPGIFQTTTGEIGAIALDFKPGTSGSPIINREGKVVGLYGNGVVTKNGGYVSGIAQANAEPEGPTPELEEEMFKKRNLTIMDLHPGSGKTRKYLPAIVREAIKRRLRTLILAPTRVVAAEMEEALKGLPIRYQTTATKSEHTGREIVVLMCHATFTMRLLSPVRVPNYNLIIMDEAHFTDPASIAARGYISTRVGMGEAAAIFMTATPPGTAEAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFMGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGERKKLRPRWLDARTYSDPLALKEFKDFAAGRK

SEQ ID NO: 14 - protein, 617 aa, Zika virus
SGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPTRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRYGEKRVLKPRWMDARVCSDHAALKSFKEFAAGKR

SEQ ID NO: 15 - protein, 166 aa, Dengue virus
FHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRTETWMSSEGAWRQIQKVETWALRHPGFTVIALFLAHAIGTSITQKGIIFILLMLVTPSMA

SEQ ID NO: 16 - protein, 166 aa, Dengue virus
FHLTTRNGEPHMIVSRQEKGKSLLFKTEDGVNMCTLMAMDLGELCEDTVTYNCPLLRQNEPEDIDCWCNSTSTWVTYGTCTATGEHRREKRSVALVPHVGMGLETRTETWMSSEGAWKHAQRIETWILRHPGFTIMAAILAYTIGTTYFQRVLIFILLTAVAPSMT

SEQ ID NO: 17 - protein, 166 aa, Dengue virus
FHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMT

SEQ ID NO: 18 - protein, 166 aa, Dengue virus
FHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAHMIGQTGIQRTVFFVLMMLVAPSYG

SEQ ID NO: 19 - protein, 168 aa, Zika virus
AEVTRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 20 - protein, 75 aa, Dengue virus
SVALAPHVGLGLETRTETWMSSEGAWRQIQKVETWALRHPGFTVIALFLAHAIGTSITQKGIIFILLMLVTPSMA

SEQ ID NO: 21 - protein, 75 aa, Dengue virus
SVALVPHVGMGLETRTETWMSSEGAWKHAQRIETWILRHPGFTIMAAILAYTIGTTYFQRVLIFILLTAVAPSMT

SEQ ID NO: 22 - protein, 75 aa, Dengue virus
SVALAPHVGMGLDTRTQTWMSAEGAWRQVEKVETWALRHPGFTILALFLAHYIGTSLTQKVVIFILLMLVTPSMT

SEQ ID NO: 23 - protein, 75 aa, Dengue virus
SVALTPHSGMGLETRAETWMSSEGAWKHAQRVESWILRNPGFALLAGFMAHMIGQTGIQRTVFFVLMMLVAPSYG

SEQ ID NO: 24 - protein, 75 aa, Zika virus
AVTLPSHSTRKLQTRSQTWLESREYTKHLIRVENWIFRNPGFALAAAAIAWLLGSSTSQKVIYLVMILLIAPAYS

SEQ ID NO: 25 - protein, 10 aa, Dengue virus
KTVWFVPSIK

SEQ ID NO: 26 - protein, 9 aa, Dengue virus
TTDISEMGA

SEQ ID NO: 27 - protein, 10 aa, Dengue virus
FVVTTDISEM

SEQ ID NO: 28 - protein, 9 aa, Dengue virus
AARGYISTR

SEQ ID NO: 29 - protein, 9 aa, Dengue virus
RTLILAPTR

SEQ ID NO: 30 - protein, 9 aa, Dengue virus
LMRRGDLPV

SEQ ID NO: 31 - protein, 9 aa, Dengue virus
EAKMLLDNI

SEQ ID NO: 32 - protein, 9 aa, Dengue virus
EIVDLMCHA

SEQ ID NO: 33 - DNA, 2116 bp, Artificial Sequence
  Description of Artificial Sequence Synthetic polynucleotide
gatatcgcaccatggatgccatgaagagaggactgtgttgcgtgctgctgctgtgtggcgccgtgttcgtgagccccttccatttaaccacacgcaatggagaaccacacatgatcgtcagcagacaagagaaagggaaaagtctcttgtttaaaacagaggatggtgtgaacatgtgtaccctcatggccatggaccttggtgaactgtgtgaagacacagtcacttataactgtcctcttctcaggcagaatgaaccagaagacatagactgctggtgcaactccacgtccacatgggtaacttatgggacatgtactgccacaggagaacacagaagggaaaaaagatcagtggcactcgtcccacatgtgggaatgggattggagacacgaactgaaacatggatgtcatcagaaggggcctggaaacacgcccagagaattgaaacctggatcttgagacatccaggttttaccataatggcagcaatcttggcatacaccataggaacaacatatttccaaagagtcctgattttcatcttactgacagctgtcgctccttcaatgacaatgcgctgcataggaatatcaaacagagactttgtggaaggggtttcaggaggaagctgggttgacatagtcttagaacatggaagctgtgtgacaacaatggcaaaaaacaaaccaacattggactttgagctgataaaaacagaagccaagcaacctgccactctaaggaagtactgtatagaggcaaagctgaccaacacaacaacagcatctcgatgcccaacacaaggggaacccagcctaaacgaagagcaggacaaaaggttcgtctgcaaacattccatggtagacagaggatggggaaatggatgtggattatttggaaaaggaggcatcgtgacctgtgccatgttcacatgcaaaaaaaacatggaagggaaaatcgtgcaaccagaaaacctggaatacaccattgtgataacacctcactcaggggaagagaatgcagttggaaatgacacaggaaaacatggcaaggaaatcaaagtaacaccacagagttctattacagaagcagaactgacaggctatggcaccgtcacgatggagtgctctccgagaacgggccttgacttcaatgagatggtgttgctgcaaatggaaaacaaagcttggttggtgcacaggcaatggttcttagacctgccgttaccatggctgcccggagcggacacacaaggatcaaattggatacagaaggagacattggtcaccttcaaaaatccccatgcaaagaaacaggatgttgttgttttaggatctcaagaaggggctatgcatacagcacttacaggggccacggaaatccagatgtcatcaggaaacctactgttcacaggacatctcaagtgcagactgagaatggacaaactacagctcaaaggaatgtcatactctatgtgtacagggaagtttaaagttgtaaaggaaatagcagaaacacaacatggaacaatagttatcagagtacaatatgaaggggatggttctccatgtaaaatcccttttgagataatggacttggaaaaaagacatgtcttaggtcgcctgattacagtcaacccaattgtcacagaaaaggacagcccagtcaacatagaagcagaacctccatttggagacagctacatcattataggagtagaaccgggacaactgaagctcagctggtttaagaaaggaagctcaatcggccaaatgttcgagacaacaatgagaggagcgaagagaatggctattttaggtgacacagcctgggattttggatccctgggaggagtgttcacatctataggaaaggccctccaccaggtttttggagcaatctatggggctgccttcagcggggtctcatggactatgaaaatcctcataggagttgtcatcacatggataggaatgaattcacgcagcacctcactgtccgtgtcgttagtattagtgggagtcgtgacattgtatttgggagtcatggtgcaggcttgattaattgatcgatacagcagcaattggcaagctgcttacatagaaggcgcgcc

SEQ ID NO: 34 - protein, 683 aa, Artificial Sequence
  Description of Artificial Sequence Synthetic polypeptide
MDAMKRGLCCVLLLCGAVFVSPFHLTTRNGEPHMIVSRQEKGKSLLFKTEDGVNMCTLMAMDLGELCEDTVTYNCPLLRQNEPEDIDCWCNSTSTWVTYGTCTATGEHRREKRSVALVPHVGMGLETRTETWMSSEGAWKHAQRIETWILRHPGFTIMAAILAYTIGTTYFQRVLIFILLTAVAPSMTMRCIGISNRDFVEGVSGGSWVDIVLEHGSCVTTMAKNKPTLDFELIKTEAKQPATLRKYCIEAKLTNTTTASRCPTQGEPSLNEEQDKRFVCKHSMVDRGWGNGCGLFGKGGIVTCAMFTCKKNMEGKIVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQA

SEQ ID NO: 35 - DNA, 2149 bp, Artificial Sequence
  Description of Artificial Sequence Synthetic polynucleotide
gatatcgcaccatggatgccatgaagagaggactgtgttgcgtgctgctgctgtgtggcgccgtgttcgtgagccccgcggaggtcactagacgtgggagtgcatactatatgtacttggacagaagcgatgctggggaggccatatcttttccaaccacactggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcagacctggttggagtcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagatacccaatatgtctgcaaaagaacgttagtggacagaggctggggaaatggatgtggactttttggaaaagggagcctggtgacatgcgctaagtttgcatgctccaagaaaatgaccgggaagagcatccagccagagaatctggagtaccggataatgctgtcagtccatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggacaaacttagattgaagggcgtgtcatactccttgtgtaccgcagcgttcacattcaccaagatcccggctgaaacactgcacgggacagtcacagtggaggtacagtacgcagggacagatggaccctgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagtaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggcctgaacacaaagaatggatctatttcccttatgtgcttggccttagggggagtgttgatcttcttatccacagccgtctctgcttgattaattgatcgatacagcagcaattggcaagctgcttacatagaaggcgcgcc

SEQ ID NO: 36 - protein, 694 aa, Artificial Sequence
  Description of Artificial Sequence Synthetic polypeptide
MDAMKRGLCCVLLLCGAVFVSPAEVTRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 37 - DNA, 2116 bp, Artificial Sequence
  Description of Artificial Sequence Synthetic polynucleotide
gatatcgcaccatggatgccatgaagagaggactgtgttgcgtgctgctgctgtgtggcgccgtgttcgtgagcccctttcacctgactacccggaacggcgagccacacatgatcgtgagcagacaggagaagggcaagagcctcctgtttaaaaccgaggacggagtgaacatgtgcactctgatggccatggacctcggagagctgtgtgaggacaccgtgacctacaattgccctctgctgcgccagaacgagcctgaggacatcgattgttggtgcaactctacttctacctgggtgacctacggaacatgtaccgccacaggcgagcataggcgagaaaaacgctctgtggccctggtgcctcacgtgggcatgggcctggagaccaggacagagacatggatgagctctgaaggcgcttggaagcacgcacagcggatcgagacatggatcctgcggcatcctgggtttactatcatggccgctatccttgcctatactatcggcacaacatactttcagagggtgctgatctttatcctgctgacagccgtggccccctctatgaccatgaggtgtattggcatcagcaacagagactttgtagagggcgtgtccgggggcagctgggtggacatcgtgctggagcacgggagctgcgtgactactatggccaaaaacaagcctacactggacttcgagctgatcaagaccgaggccaagcaacccgccacactgaggaagtactgcatcgaggccaagctgaccaacaccaccaccgctagcagatgtcctcgcgagggcgaaccttcactgaacgaggagcaggacaagagattcgtgtgcaagcacagcatggtggacagagggcgggggaatggctgtggcaggttcgggaagggcgggatcgtgacatgcgccatgtttacatgcaagaagaacatggagggcaaaatcgtccagccagagaacctggaatataccatcgtgatcacccctcacagcggagaggagaacgctgtgggaaacgacacaggcaagcatgggaaggaaattaaggtgactccacagagcagcatcacagaggctgagctgaccggctatggcaccgtgaccatggagtgcagcccaagaaccggcctcgatttcaatgagatggtcctgctgcagatggaaaacaaagcctggctggtgcacagacagtggttcctggacctccccctgccttggctgccaggagccgacacccaggggtccaactggatccagaaagagaccctggtgacattcaagaaccctcacgcaaaaaagcaggacgtggtggtgctcggaagccaggagggcgctatgcataccgctctgaccggcgccacagagattcagatgagcagcggcaatctgctcttcaccggacacctgaagtgtcggctgaggatggacaagctgcagctgaagggaatgtcctactccatgtgcaccggcaagttcaaggtggtgaaggaaattgctgagacccagcacggcaccatcgtgattagggtgcagtatgagggtgacggaagcccatgcaagatcccattcgaaatcatggatctggagaagcggcacgtgctggggagactgatcaccgtgaatcctatcgtgaccgagaaagactcccccgtgaacatcgaagccgagccacctttcggggattcttatatcatcattggcgtggagcctggccagctgaagctgtcctggttcaagaagggaagctctatcggacagatgttcgagacaaccatgagaggcgccaagcggatggccatcctgggtgacactgcctgggatttcgggagcctcggcggcgtgttcactagtattggcaaggctctgcatcaagtgttcggcgccatctacggcgctgcattcagcggcgtctcctggacaatgaaaattctgatcggcgtggtgatcacatggataggcatgaacagcaggtctacctctctgagcgtctctctggtgctggtcggggtggtgacactgtatctgggcgtgatggtgcaggcctgattaattgatcgatacagcagcaattggcaagctgcttacatagaaggcgcgcc

SEQ ID NO: 38 - protein, 683 aa, Artificial Sequence
  Description of Artificial Sequence Synthetic polypeptide
MDAMKRGLCCVLLLCGAVFVSPFHLTTRNGEPHMIVSRQEKGKSLLFKTEDGVNMCTLMAMDLGELCEDTVTYNCPLLRQNEPEDIDCWCNSTSTWVTYGTCTATGEHRREKRSVALVPHVGMGLETRTETWMSSEGAWKHAQRIETWILRHPGFTIMAAILAYTIGTTYFQRVLIFILLTAVAPSMTMRCIGISNRDFVEGVSGGSWVDIVLEHGSCVTTMAKNKPTLDFELIKTEAKQPATLRKYCIEAKLTNTTTASRCPREGEPSLNEEQDKRFVCKHSMVDRGRGNGCGRFGKGGIVTCAMFTCKKNMEGKIVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQA

SEQ ID NO: 39 - DNA, 2149 bp, Artificial Sequence
  Description of Artificial Sequence Synthetic polynucleotide
gatatcgcaccatggatgccatgaagagaggactgtgttgcgtgctgctgctgtgtggcgccgtgttcgtgagccccgctgaagtgaccagaagaggctccgcttactacatgtatctggaccggagcgatgccggcgaagctattagcttcccaaccaccctgggcatgaataagtgttacattcagatcatggacctgggccacatgtgcgacgccaccatgtcttacgagtgtcctatgctggacgagggcgtggagcccgacgacgtggactgctggtgcaatactaccagcacctgggtggtgtacggcacctgccaccacaagaagggagaggccagacgctctaggagagcagtgaccctgccctcccactccacaaggaagctgcagacccggtcccagacctggctcgagtctagggagtacactaagcacctgatcagagtcgagaactggatctttagaaatcccgggttcgccctggccgccgccgccatcgcctggctcctggggtccagcacctctcagaaagtgatctatctggtgatgatcctgctgattgcccccgcttacagcatcagatgtatcggagtgagcaacagagacttcgtggagggcatgagcggcgggacatgggtggacgtggtgctggagcacggcggctgtgtgaccgtgatggcccaggacaagcccactgttgacatcgagctggtcaccaccaccgtgagcaacatggccgaggtgcggtcatactgctacgaggccagcatcagcgatatggcctctgacagtaggtgcccaagagagggcgaggcctatctggacaagcagtctgacacccagtacgtctgtaagcgcacccttgtggataggggtaggggcaatggctgcggcagatttggtaagggctccctggtaacttgtgcaaagtttgcttgcagtaagaagatgacaggcaagtctatccagcccgagaacctggaatacagaattatgctgtccgtgcacggcagtcagcatagcggaatgatcgtgaacgacaccggacacgagacagatgagaacagggccaaggtcgagatcacacctaactcccccagggccgaggccaccctgggcgggttcggatctctgggcctggactgcgagcctagaacaggcctggacttcagcgacctgtattatctgacaatgaataacaagcactggctggtgcacaaggagtggttccacgacatccctctgccctggcacgccggagctgacaccgggacccctcactggaacaataaggaggcactggttgagttcaaggacgcccacgccaagcggcagaccgtggtggttctgggtagccaggagggcgccgtgcacaccgcactggctggcgccctcgaggctgaaatggatggcgccaagggaaggctgagttccggtcacctgaagtgtaggctgaagatggacaagctgaggctcaagggcgtgtcctacagcctgtgcacagccgccttcacattcaccaaaatccctgccgaaacactccacgggaccgtgaccgtggaggtgcagtacgccgggaccgatggcccatgcaaggtgcctgcacagatggctgtggatatgcagactctgacccctgtggggagactgattaccgccaatcccgtgatcactgaatcaactgagaacagcaagatgatgctggagctggatccacctttcggcgactcatacatcgtgattggcgtgggcgagaaaaagatcacccaccattggcaccgctctggcagtaccatcggcaaggccttcgaggcaacagtgagaggcgccaagaggatggccgtgctgggagataccgcttgggacttcggctcagtgggaggcgctctgaacagcctcggaaagggcatccatcagatcttcggcgcagctttcaagtctctgttcgggggcatgtcatggttcagccagattctgatcggtacactgctgatgtggctggggctgaatactaagaacgggagcatctccctgatgtgcctggctctgggaggggtgctgatcttcctcagcaccgccgtgagcgcctgattaattgatcgatacagcagcaattggcaagctgcttacatagaaggcgcgcc

SEQ ID NO: 40 - protein, 694 aa, Artificial Sequence
  Description of Artificial Sequence Synthetic polypeptide
MDAMKRGLCCVLLLCGAVFVSPAEVTRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPREGEAYLDKQSDTQYVCKRTLVDRGRGNGCGRFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 41 - DNA, 1990 bp, Artificial Sequence
  Description of Artificial Sequence Synthetic polynucleotide
gatatcgcaccatggatgccatgaagagaggactgtgttgcgtgctgctgctgtgtggcgccgtgttcgtgagcccctccggcgttctatgggacgtacccagccccccagagacacagaaagcagaactggaagagggggtctataggatcaaacagcaaggaatttttgggaaaacccaagtaggggttggagtacagaaagaaggagtcttccacaccatgtggcacgttacaagaggggcggtgttgacatataatgggaaaagactggaaccaaactgggctagtgtgaaaaaagatctgatttcatacggaggaggatggagattgagcgcacaatggcaaaagggggaggaggtgcaggttattgccgtagagcctgggaagaacccaaagaactttcaaaccatgccaggcacttttcagactacaacaggggaaataggagcaattgcactggatttcaagcctggaacttcaggatctcctatcataaacagagagggaaaggtagtgggactgtatggcaatggagtggttacaaagaatggtggctatgtcagcggaatagcgcaaacgaatgcagaaccagatggaccgacaccagaattggaagaagagatgttcaaaaagcgaaatctaaccataatggatcttcatcctgggtcaggaaagacacggaaataccttccagctattgttagagaggcaatcaagagacgtctaagaactctaattttggcaccgacaagggtggttgcagctgagatggaagaagcattgaaagggctcccaataaggtaccaaacaacagcaacaaaatctgaacacacaggaagagagattgttgatctaatgtgccacgcaacgttcacaatgcgcctgctgtcaccagttagggttccaaattataacttgataataatggatgaagcccatttcacagacccagccagtatagcggctagagggtacatatcgactcgtgttggaatgggagaggcagccgcaattttcatgacagcaacgccccctggaacagctgatgcctttcctcagagcaacgctccaattcaagatgaagaaagggacataccggaacgctcatggaattcaggcaatgaatggataaccgacttcgctgggaaaacggtgtggtttgtccccagcattaaagccggaaatgacatagcaaactgcttgcgaaaaaacgggaaaaaggtcattcaacttagtaggaagacttttgacacagaatatcagaaaactaaactgaatgattgggacttcgtagtgacaactgacatttcagaaatgggggccaatttcaaagcagatagagtgatcgacccaagaagatgtctcaaaccagtgatcctgacagatggaccagagcgggtgatcctggctggaccaatgccagtcaccgcggcgagtgctgcgcaaaggagagggagagttggcaggaacccacaaaaagaaaatgaccagtacatattcatgggccaacctctcaataatgatgaagaccacgctcactggacagaagcaaaaatgctgctggacaacattaatacaccagaagggatcataccagctctctttgagccagaaagggagaagtcagccgccatagacggtgagtatcgcttgaaaggtgaatctaggaagactttcgtggaactcatgaggaggggtgaccttccagtctggttagcccataaagtagcatcagaagggatcaaatatacagatagaaaatggtgctttgatggacaacgtaataatcaaattttagaggagaacatggatgtggaaatctggacaaaggaaggagaaaagaaaaaattgagacctaggtggcttgatgcccgcacttattcagatcccttagcactcaaggaattcaaggactttgcggctggcagaaagtgattaattgatcgatacagcagcaattggcaagctgcttacatagaaggcgcgcc

SEQ ID NO: 42 - protein, 641 aa, Artificial Sequence
  Description of Artificial Sequence Synthetic polypeptide
MDAMKRGLCCVLLLCGAVFVSPSGVLWDVPSPPETQKAELEEGVYRIKQQGIFGKTQVGVGVQKEGVFHTMWHVTRGAVLTYNGKRLEPNWASVKKDLISYGGGWRLSAQWQKGEEVQVIAVEPGKNPKNFQTMPGTFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFMGQPLNNDEDHAHWTEAKMLLDNINTPEGIIPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRK

SEQ ID NO: 43 - DNA, 1990 bp, Artificial Sequence
  Description of Artificial Sequence Synthetic polynucleotide
gatatcgcaccatggatgccatgaagagaggactgtgttgcgtgctgctgctgtgtggcgccgtgttcgtgagcccctccggagtcctgtgggacgtgcctagcccacccgaaacccagaaggctgagctggaggagggcgtgtacagaattaagcagcagggcatttttggcaagacccaggtgggggtgggcgtgcagaaggagggggtgttccacaccatgtggcacgtgactcgaggagcagtgctcacacacaacggcaagaggctggagcccaactgggcttccgtgaagaaggacctgatttcttatggcggagggtggagactgtccgcccagtggcagaagggagaggaggtgcaggtgatcgccgtggaacccggcaagaacccaaagaacttccagaccatgcctggcacattccagacaaccacaggcgagatcggagctatcgctctggacttcaagcctggcacctccggctctcccatcatcaacaaggagggcaaagtggtggggctgtacggaaacggcgtcgtgaccaagaacggcggctatgtgagcggcatcgcccagacaaacgccgaacctgacggccctactcccgagctagaggaggaaatgttcaagaaacgcaacctgacaatcatggacctgcaccctgggtccggcaaaacaaggaagtacctgcccgccatcgtgcgcgaggctatcaagaggagactgaggaccctgatcctggccccaacaagagtggtggccgccgagatggaagaggctctgaagggcctgcccatcagataccagaccaccgcaaccaagagcgagcacaccggcagggaaatcgtggacctcatgtgtcatgccacctttaccatgcgcctgctgtctccagtgagggtgcctaactacaacctgatcatcatggacgaggcccatttcaccgaccctgcctccatcgctgcccggggctacatcagcacccgggtgggcatgggcgaggctgccgccatcttcatgacagctaccccaccaggtacagccgatgccttccctcagagtaacgccccaatccaggacgaagaaagagacatccctgaaagatcatggaatagcggcaacgaatggatcacagactttgccggaaagaccgtgtggttcgtgccttccatcaaggctggcaatgacatcgctaactgtctgaggaaaaatggaaagaaggtgatccagctgagcaggaagacatttgacacagagtaccagaagacaaagctgaacgactgggacttcgtcgtgaccacagacatcagcgaaatgggagccaacttcaaggccgacagggtgatcgaccccaggcggtgcctgaaacctgtcatcctgacagacggccccgagcgggtgatcctggcggggccaatgcccgtgactgccgccagcgcagctcagagacgcggcagagtgggccggaacccccagaaggaaaacgaccagtacatcttcacaggccagcccctgaacaatgacgaggatcacgcccattggaccgaagccaaaatgctgctggacaacattaacacccctgagggcatcctgcccgccctgttcgagcctgagagagagaaatccgccgccatcgatggcgagtaccgactgaaaggggagtccagaaagacatttgtggaactgatgcggagaggcgacctgcccgtgtggctggcccacaaggtcgctagcgagggaatcaaatacacagaccgcaagtggtgcttcgatggccagaggaataatcagatcctggaggagaatatggatgtggagatttggactaaggagggcgagaagaagaagctgaggccacgatggctggacgccaggacatacagcgaccctctggctctgaaggagttcaaagacttcgccgcaggcaggaagtgattaattgatcgatacagcagcaattggcaagctgcttacatagaaggcgcgcc

SEQ ID NO: 44 - protein, 641 aa, Artificial Sequence
  Description of Artificial Sequence Synthetic polypeptide
MDAMKRGLCCVLLLCGAVFVSPSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGTFQTTTGEIGAIALDFKPGTSGSPIINKEGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGILPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRK

SEQ ID NO: 45 - DNA, 1990 bp, Artificial Sequence
  Description of Artificial Sequence Synthetic polynucleotide
gatatcgcaccatggatgccatgaagagaggactgtgttgcgtgctgctgctgtgtggcgccgtgttcgtgagcccctccggagtcctgtgggacgtgcctagcccacccgaaacccagaaggctgagctggaggagggcgtgtacagaattaagcagcagggcatttttggcaagacccaggtgggggtgggcgtgcagaaggagggggtgttccacaccatgtggcacgtgactcgaggagcagtgctcacacacaacggcaagaggctggagcccaactgggcttccgtgaagaaggacctgatttcttatggcggagggtggagactgtccgcccagtggcagaagggagaggaggtgcaggtgatcgccgtggaacccggcaggaacccaaagaacttccagaccatgcctggcatattccagacaaccacaggcgagatcggagctatcgctctggacttcaagcctggcacctccggctctcccatcatcaacagggagggcaaagtggtggggctgtacggaaacggcgtcgtgaccaagaacggcggctatgtgagcggcatcgcccaggcaaacgccgaacctgagggccctactcccgagctagaggaggaaatgttcaagaaacgcaacctgacaatcatggacctgcaccctgggtccggcaaaacaaggaagtacctgcccgccatcgtgcgcgaggctatcaagaggagactgaggaccctgatcctggccccaacaagagtggtggccgccgagatggaagaggctctgaagggcctgcccatcagataccagaccaccgcaaccaagagcgagcacaccggcagggaaatcgtggtgctcatgtgtcatgccacctttaccatgcgcctgctgtctccagtgagggtgcctaactacaacctgatcatcatggacgaggcccatttcaccgaccctgcctccatcgctgcccggggctacatcagcacccgggtgggcatgggcgaggctgccgccatcttcatgacagctaccccaccaggtacagccgaggccttccctcagagtaacgccccaatccaggacgaagaaagagacatccctgaaagatcatggaatagcggcaacgaatggatcacagactttgtgggaaagaccgtgtggttcgtgccttccatcaaggctggcaatgacatcgctaactgtctgaggaaaaatggaaagaaggtgatccagctgagcaggaagacatttgacacagagtaccagaagacaaagctgaacgactgggacttcgtcgtgaccacagacatcagcgaaatgggagccaacttcaaggccgacagggtgatcgaccccaggcggtgcctgaaacctgtcatcctgacagacggccccgagcgggtgatcctggcggggccaatgcccgtgactgccgccagcgcagctcagagacgcggcagagtgggccggaacccccagaaggaaaacgaccagtacatcttcatgggccagcccctgaacaatgacgaggatcacgcccattggaccgaagccaaaatgctgctggacaacattaacacccctgagggcatcatccccgccctgttcgagcctgagagagagaaatccgccgccatcgatggcgagtaccgactgaaaggggagtccagaaagacatttgtggaactgatgcggagaggcgacctgcccgtgtggctggcccacaaggtcgctagcgagggaatcaaatacacagaccgcaagtggtgcttcgatggcgaaaggaataatcagatcctggaggagaatatggatgtggagatttggactaaggagggcgagagaaagaagctgaggccacgatggctggacgccaggacatacagcgaccctctggctctgaaggagttcaaagacttcgccgcaggcaggaagtgattaattgatcgatacagcagcaattggcaagctgcttacatagaaggcgcgcc

SEQ ID NO: 46 - protein, 641 aa, Artificial Sequence
  Description of Artificial Sequence Synthetic polypeptide
MDAMKRGLCCVLLLCGAVFVSPSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGRNPKNFQTMPGIFQTTTGEIGAIALDFKPGTSGSPIINREGKVVGLYGNGVVTKNGGYVSGIAQANAEPEGPTPELEEEMFKKRNLTIMDLHPGSGKTRKYLPAIVREAIKRRLRTLILAPTRVVAAEMEEALKGLPIRYQTTATKSEHTGREIVVLMCHATFTMRLLSPVRVPNYNLIIMDEAHFTDPASIAARGYISTRVGMGEAAAIFMTATPPGTAEAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFMGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGERKKLRPRWLDARTYSDPLALKEFKDFAAGRK

SEQ ID NO: 47 - DNA, 1984 bp, Artificial Sequence
  Description of Artificial Sequence Synthetic polynucleotide
gatatcgcaccatggatgccatgaagagaggactgtgttgcgtgctgctgctgtgtggcgccgtgttcgtgagccccagtggtgctctatgggatgtgcctgctcccaaggaagtaaaaaagggggagaccacagatggagtgtacagagtaatgactcgtagactgctaggttcaacacaagttggagtgggagtcatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggactacccagcaggaacttcaggatctccaatcctagacaagtgtgggagagtgataggactctatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaactagggttgtcgctgctgaaatggaggaagcccttagagggcttccagtgcgttatatgacaacagcagtcaatgtcacccactctgggacagaaatcgttgacttaatgtgccatgccaccttcacttcacgtctactacagccaaccagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccggagagagcctggagctcaggctttgattgggtgacggaccattctggaaaaacagtttggtttgttccaagcgtgaggaacggcaatgagatcgcagcttgtctgacgaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgatgaagaccatgcacactggcttgaagcaagaatgctccttgacaatatctacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtctgttcagatcatgcggccctgaagtcattcaaggagtttgccgctgggaaaagatgattaattgatcgatacagcagcaattggcaagctgcttacatagaaggcgcgcc

SEQ ID NO: 48 - protein, 639 aa, Artificial Sequence
  Description of Artificial Sequence Synthetic polypeptide
MDAMKRGLCCVLLLCGAVFVSPSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPTRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRYGEKRVLKPRWMDARVCSDHAALKSFKEFAAGKR

SEQ ID NO: 49 - protein, 9 aa, Dengue virus
SPCKIPFEI

(19) United States
(12) Patent Application Publication
(10) Pub. No.: US 2023/0149526 A1
(43) Pub. Date: May 18, 2023

(54) COMBINATIONS OF FLAVIVIRUS PROTEINS, PEPTIDE SEQUENCES, EPITOPES, AND METHODS AND USES THEREOF

(71) Applicant: LA JOLLA INSTITUTE FOR IMMUNOLOGY, La Jolla, CA (US)

(72) Inventors: Sujan SHRESTA, La Jolla, CA (US); Jason GREENBAUM, La Jolla, CA (US)

(21) Appl. No.: 17/841,267

(22) Filed: Jun. 15, 2022

Related U.S. Application Data
(63) Continuation-in-part of application No. PCT/US2020/065368, filed on Dec. 16, 2020.
(60) Provisional application No. 62/948,653, filed on Dec. 16, 2019.

(51) Int. Cl. A61K 39/12; C07K 14/18; A61P 31/14; C07K 14/705; A61K 39/39; A61P 37/04

(52) U.S. Cl. CPC A61K 39/12; C07K 14/1825; A61P 31/14; C07K 14/70503; A61K 39/39; A61P 37/04; C12N 2770/24122; C12N 2770/24134

(57) ABSTRACT

The present application relates to composition of matter, processes and use of composition of matter relating to flavivirus proteins, peptides, and epitopes, for example, for therapeutic or preventative vaccination against one or more flavivirus serotypes or species, and/or for inducing, enhancing, or sustaining an immune response against at least one flavivirus serotype or species. The flavivirus may be for example the Zika and/or Dengue virus.

### CROSS-REFERENCE

This application is a continuation-in-part application of PCT/US2020/065368 filed Dec. 16, 2020, which claims the benefit of U.S. Provisional Application No. 62/948,653, filed Dec. 16, 2019, both of which are incorporated herein by reference in its entirety.

### TECHNICAL FIELD

The present application relates to compositions of matter, processes and use of compositions of matter relating to flavivirus proteins, peptides, and epitopes.

### BACKGROUND

Flavivirus is a genus of viruses in the family Flaviviridae. This genus includes the West Nile virus, dengue virus (DENV), tick-borne encephalitis (TBE) virus, yellow fever virus, Zika virus (ZIKV) and several other viruses which may cause encephalitis (e.g. Japanese encephalitis), as well as insect-specific flaviviruses (ISFs) such as cell fusing agent virus (CFAV), Palm Creek virus (PCV), and Parramatta River virus (PaRV).

Flaviviruses share several common aspects: common size (40-65 nm), symmetry (enveloped, icosahedral nucleocapsid), nucleic acid (positive-sense, single-stranded RNA around 10,000-11,000 bases), and appearance in the electron microscope. Flaviviruses are globally emerging and cause significant human disease in the form of encephalitis or hemorrhagic fever. Most flaviviruses are maintained in animal reservoirs in nature and are transmitted to humans primarily through the bite of an infected mosquito or tick. Other virus transmission routes can include handling infected animal carcasses, blood transfusion, child birth and through consumption of unpasteurized milk products.

There are, however, fundamental gaps in the understanding of flaviviruses immunology and pathogenesis.

Vaccines are currently available for only yellow fever and Japanese and TBE; however, new vaccines for dengue and West Nile are in clinical trials in humans. In recent years, many studies have shown that flaviviruses, especially dengue virus has the ability to inhibit the innate immune response during the infection (Diamond M S (September 2009), J. Interferon Cytokine Res. 29 (9): 521-30; Jones M, Davidson A, Hibbert L, et al. (May 2005). J. Virol. 79 (9): 5414-20). Indeed, the dengue virus has many nonstructural proteins that allow the inhibition of various mediators of the innate immune system response. Disease diagnosis can be difficult as all flaviviruses are antigenically and genetically closely related. There are no effective antiviral therapies that exist for any flavivirus so the main approach to disease control is through vaccination and vector control.

In recent years, there has been a rise in the spread of Zika infection. Most cases of ZIKA infection have no symptoms, but when present they are usually mild and can resemble dengue fever, and may cause fever, rash, headache, pain behind the eyes, conjunctivitis, muscle or joint pain, nausea, vomiting, or loss of appetite. However, a causal relationship between ZIKV and a congenital syndrome including microcephaly has been confirmed in the 2015 Brazilian outbreak, and signs of microcephaly have been seen in ZIKV-infected mice. ZIKV has also been linked to Guillain-Barre Syndrome (GBS) and case reports of sexual transmission are mounting. Recently there was a major outbreak of ZIKV in the Western Hemisphere, which also was associated with GBS. Additionally, infection of pregnant women was confirmed to cause Congenital ZIKV Syndrome, which includes microcephaly and other birth defects. (Mlakar, J., et al., Zika Virus Associated with Microcephaly. N Engl J Med, 2016. 374(10): p. 951-8; Driggers, R. W., et al., Zika Virus Infection with Prolonged Maternal Viremia and Fetal Brain Abnormalities. N Engl J Med, 2016. 374(22): p. 2142-51; Hennessey, M., M. Fischer, and J. E. Staples, Zika Virus Spreads to New Areas—Region of the Americas, May 2015-January 2016. MMWR Morb Mortal Wkly Rep, 2016. 65(3): p. 55-8; Rasmussen, S. A., et al., Zika Virus and Birth Defects—Reviewing the Evidence for Causality. N Engl J Med, 2016. 374(20): p. 1981-7).

As mosquito control has failed, and with the new disease syndromes caused by and associated with DENV and ZIKV infection, there is an urgent need to address the fundamental gaps in the understanding of flaviviruses immunology and pathogenesis so as to be able to develop more effective flavivirus vaccines and/or treatment approaches.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a composition comprising a protein or peptide, or variant, homologue, derivative or subsequence thereof, that comprises, consists or consists essentially of a flavivirus B cell epitope, or a nucleic acid molecule encoding the protein or peptide, or variant, homologue, derivative or subsequence thereof; and a protein or peptide, or variant, homologue, derivative or subsequence thereof, that comprises, consists or consists essentially of a flavivirus T cell epitope or a nucleic acid molecule encoding the protein or peptide, or variant, homologue, derivative or subsequence thereof; wherein the composition elicits, stimulates, induces, promotes, increases or enhances an antibody response and a T cell response against two or more different serotypes of a flavivirus or two or more different species of flavivirus.

In one non-limiting embodiment, the composition elicits, stimulates, induces, promotes, increases, or enhances an antibody response against two or more different serotypes of a flavivirus and two or more different species of flavivirus and a T cell response against two or more different serotypes of a flavivirus and two or more different species of flavivirus. In further embodiments, the protein or peptide, or variant, homologue, derivative, or subsequence thereof, that comprises, consists, or consists essentially of a flavivirus B cell epitope, is a flavivirus envelope, membrane, premembrane, core, NS1, NS2A, NS2B, NS3, NS4A, NS4B or NS5 protein or peptide. In still other embodiments, the protein or peptide, or variant, homologue, derivative, or subsequence thereof, that comprises, consists, or consists essentially of a flavivirus T cell epitope, is a flavivirus envelope, membrane, premembrane, core, NS1, NS2A, NS2B, NS3, NS4A, NS4B or NS5 protein or peptide

In one non-limiting embodiment, the composition comprises two or more proteins or peptides, or variants, homologues, derivatives or subsequences thereof, from two or more different serotypes of a flavivirus or two or more different species of flavivirus, or nucleic acid molecules encoding two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus B cell epitope. In further embodiments, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus B cell epitope. In still other embodiments, the composition comprises two or more proteins or peptides, or variants, homologues, derivatives or subsequences thereof, from two or more different serotypes of a flavivirus or two or more different species of flavivirus, or nucleic acid molecules encoding the two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus T cell epitope.

In one non-limiting embodiment, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus T cell epitope

In certain non-limiting embodiments, the composition comprises proteins or peptides, or variants, homologues, derivatives, or subsequences thereof from DENV 1-4 Dengue virus serotypes, or nucleic acid molecules encoding the proteins or peptides, or variants, homologues, derivatives, or subsequences thereof, from DENV 1-4 Dengue virus serotypes. In further embodiments, the composition comprises proteins or peptides, or variants, homologues, derivatives, or subsequences thereof from five Dengue virus serotypes, or nucleic acid molecules encoding the proteins or peptides, or variants, homologues, derivatives, or subsequences thereof, from five Dengue virus serotypes. In certain alternative embodiments, the composition further comprises a protein, or variant, homologue, derivatives, or subsequence thereof from Zika virus, or nucleic acid molecules encoding the protein, or variant, homologue, derivative, or subsequence thereof, from Zika virus.

In certain non-limiting embodiments, the composition comprises the premembrane and envelope proteins or peptides, or variants, homologues, derivatives, or subsequences thereof, from DENV 1-4 Dengue virus serotypes, or nucleic acid molecules encoding the premembrane and envelope proteins or peptides, or variants, homologues, derivatives, or subsequences thereof, from DENV 1-4 Dengue virus serotypes. In other embodiments, the composition comprises the premembrane and envelope proteins or peptides, or variants, homologues, derivatives, or subsequences thereof, from five Dengue virus serotypes, or nucleic acid molecules encoding the premembrane and envelope proteins or peptides, or variants, homologues, derivatives, or subsequences thereof, from five Dengue virus serotypes. In certain alternative embodiments, the composition comprises the premembrane and envelope proteins or peptides, or a variants, homologues, derivatives or subsequences thereof, from Zika virus, or nucleic acid molecules encoding the premembrane and envelope proteins or peptides, or variants, homologues, derivatives, or subsequences thereof, from Zika virus.

In one non-limiting embodiment, the composition comprises a protein, or variant, homologue, derivative or subsequence thereof, that comprises, consists or consists essentially of a consensus amino acid sequence derived from proteins or peptides from two or more different serotypes of a flavivirus or proteins or peptides from two or more different species of flavivirus, or nucleic acid molecules encoding the consensus amino acid sequence derived from proteins or peptides from two or more different serotypes of a flavivirus or proteins or peptides from two or more different species of flavivirus. In certain alternative embodiments, the composition comprises a protein, or variant, homologue, derivative or subsequence thereof, that comprises, consists or consists essentially of a consensus sequence derived from proteins or peptides from two or more different serotypes of a flavivirus and two or more different species of flavivirus, or nucleic acid molecules encoding the consensus sequence derived from proteins or peptides from two or more different serotypes of a flavivirus and two or more different species of flavivirus.

In one non-limiting embodiments, the composition comprises a protein, or variant, homologue, derivative or subsequence thereof, that comprises, consists or consists essentially of a consensus sequence derived from the NS3 proteins or peptides from two or more different serotypes of a flavivirus or two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus or two or more different species of flavivirus. In certain alternative embodiments, the composition comprises a protein, or variant, homologue, derivative or subsequence thereof, that comprises, consists or consists essentially of a consensus sequence derived from the NS3 proteins or peptides from two or more different serotypes of a flavivirus and two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus and two or more different species of flavivirus.

In one non-limiting embodiment, the composition comprises a protein, or variant, homologue, derivative or subsequence thereof, that comprises, consists or consists essentially of a consensus sequence derived from the NS3 proteins or peptides from DENV 1-4 serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from DENV 1-4serotypes of Dengue virus and Zika virus. In certain alternative embodiments, the composition comprises a protein, or variant, homologue, derivative or subsequence thereof, that comprises, consists or consists essentially of a consensus sequence derived from the NS3 proteins or peptides from five serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from five serotypes of Dengue virus and Zika virus.

In one non-limiting embodiment, the composition comprises a protein, or variant, homologue, derivative, or subsequence thereof, that comprises, consists, or consists essentially of one or more an amino acid sequence provided in Table 1, or a nucleic acid molecule encoding a protein, or variant, homologue, derivative, or subsequence thereof, that comprises, consists, or consists essentially of one or more an amino acid sequence provided in Table 1.

In one non-limiting embodiment, the composition further comprises a CD70 protein or peptide, or variant, homologue, derivative, or subsequence thereof, or a nucleic acid molecule encoding a CD70 protein, or variant, homologue, derivative, or subsequence thereof. In certain embodiments, the CD70 protein or peptide is a human CD70 protein or peptide, or variant, homologue, derivative, or subsequence thereof, or a nucleic acid molecule encoding a human CD70 protein, or variant, homologue, derivative, or subsequence thereof.

In one non-limiting embodiment, the flavivirus is one or more of Dengue virus, Zika virus, Yellow Fever virus, West Nile Virus, Japanese encephalitis, tick-borne encephalitis, cell fusing agent virus (CFAV), Palm Creek virus (PCV), or Parramatta River virus (PaRV).

In certain embodiments, the composition further comprises an adjuvant.

In one non-limiting embodiment, the composition comprises one or more vectors configured to direct expression of the protein, or variant, homologue, derivative, or subsequence thereof, that comprises, consists, or consists essentially of a flavivirus B cell epitope, and the protein, or variant, homologue, derivative, or subsequence thereof, that comprises, consists, or consists essentially of a flavivirus T cell epitope. In certain alternative embodiments, the composition comprises a vector configured to direct expression of the CD70 protein.

As embodied and broadly described herein, the present disclosure further relates to a method of eliciting, stimulating, inducing, promoting, increasing, or enhancing an immune response against a flavivirus, the method comprising administering the compositions disclosed herein.

In one non-limiting embodiment, the method elicits, stimulates, induces, promotes, increases, or enhances an immune response against two or more different serotypes of a flavivirus or two or more different species of flavivirus. In certain alternative embodiments, the method elicits, stimulates, induces, promotes, increases, or enhances an immune response against two or more different serotypes of a flavivirus and two or more different species of flavivirus.

As embodied and broadly described herein, a method is provided herein to vaccinate a subject against, provide a subject with protection against, or treat a subject for a flavivirus infection.

In one non-limiting example, the method vaccinates against, provides the subject with protection against or treats a subject for infection with two or more different serotypes of a flavivirus or two or more different species of flavivirus. In certain alternative embodiments, the method vaccinates against, provides the subject with protection against or treats a subject for infection with two or more different serotypes of a flavivirus and two or more different species of flavivirus.

In certain non-limiting embodiments, the method prevents, reduces, or inhibits sensitizing the subject to or occurrence in the subject of severe flavivirus disease or disease upon a secondary or subsequent flavivirus infection or following administration of the compositions disclosed herein subsequent to a prior flavivirus infection in the subject or prior to administration to the subject of a vaccine against a flavivirus.

As embodied and broadly described herein, the present disclosure further relates to method of formulating a vaccine against a flavivirus that will not elicit, stimulate, induce, promote, increase, enhance or sensitize a subject to severe flavivirus disease or infection, the method comprising formulating the vaccine to comprise the compositions disclosed herein.

In one non-limiting embodiment, the severe flavivirus disease or infection is severe Dengue disease, severe Dengue infection, severe Zika disease or severe Zika infection.

In non-limiting embodiments, the herein described method of inducing, enhancing, or sustaining an immune response against a flavivirus in a subject may afford one to obtain at least one of the following features: reduce flavivirus titer, increase or stimulate flavivirus clearance, reduce or inhibit flavivirus proliferation, reduce or inhibit increases in flavivirus titer or flavivirus proliferation, reduce the amount of a flavivirus protein or the amount of a flavivirus nucleic acid, or reduce or inhibit synthesis of a flavivirus protein or a flavivirus nucleic acid.

In one non-limiting embodiment, the herein described method of inducing, enhancing, or sustaining an immune response against a flavivirus in a subject includes contacting T cells of the subject with the effective amount of the composition of the present disclosure prior to, substantially contemporaneously with or following exposure to or infection of the subject with the flavivirus. For example, contacting T cells of the subject with the effective amount of the composition of the present disclosure may occur within 2-72 hours, 2-48 hours, 4-24 hours, 4-18 hours, or 6-12 hours after a rash develops.

In one non-limiting embodiment, the flavivirus is a Zika virus.

In one non-limiting embodiment, the flavivirus is a Dengue virus.

In the case where the flavivirus is a Zika or Dengue virus, the herein described method of inducing, enhancing, or sustaining an immune response against a flavivirus in a subject may treat or mitigate symptoms associated with a Zika and/or Dengue virus infection such as, but not limited to, fever, rash, headache, pain behind the eyes, conjunctivitis, muscle or joint pain, nausea, vomiting, or loss of appetite.

In one non-limiting embodiment, the composition of the present disclosure may include one or more acceptable carrier selected from the acceptable carriers described herein. For example, an acceptable carrier may be selected from gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors, and the like.

Additionally or alternatively, the composition of the present disclosure may include one or more pharmaceutically acceptable salt selected from the pharmaceutically acceptable salts described herein. For example, a pharmaceutically acceptable salt may be selected from sodium chloride, potassium chloride, sodium sulfate, ammonium sulfate, or sodium citrate. The concentration of the pharmaceutically acceptable salt can be any suitable concentration known in the art, and may be selected from about 10 mM to about 200 mM.

Additionally or alternatively, the composition of the present disclosure may include one or more adjuvant selected from the adjuvants described herein. In different embodiments, an adjuvant can be a naturally occurring adjuvant or a non-naturally occurring adjuvant. For example, an adjuvant may be selected from aluminum hydroxide or mineral oil, and a stimulator of immune responses, such as Bordetella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In one embodiment, adjuvants may include adjuvants which stimulate a Th1 type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvant.

Additionally or alternatively, the composition of the present disclosure and/or the method of the present disclosure may further include one or more components, such as drugs, immunostimulants (such as α-interferon, β-interferon, γ-interferon, granulocyte macrophage colony stimulator factor (GM-CSF), macrophage colony stimulator factor (M-CSF), and interleukin 2 (IL2)), antioxidants, surfactants, flavoring agents, volatile oils, buffering agents, dispersants, propellants, and preservatives.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical, and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly, or sub-cutaneously by injection, by infusion or per os. Suitable dosages will vary, depending upon factors such as the amount of each of the components in the composition, the desired effect (short or long term), the route of administration, the age, and the weight of the subject to be treated. Any other methods well known in the art may be used for administering the composition of the present disclosure.

The composition of the present disclosure may be formulated as a dry powder (i.e., in lyophilized form). Freeze-drying (also named lyophilization) is often used for preservation and storage of biologically active material because of the low temperature exposure during drying. Typically the liquid antigen is freeze dried in the presence of agents to protect the antigen during the lyophilization process and to yield a cake with desirable powder characteristics. Sugars such as sucrose, mannitol, trehalose, or lactose (present at an initial concentration of 10-200 mg/mL) are commonly used for cryoprotection of protein antigens and to yield lyophilized cake with desirable powder characteristics. Lyophilizing the composition theoretically results in a more stable composition.

In certain embodiments, the composition of the present disclosure may be formulated as a liquid (e.g. aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Where the composition of the present disclosure is intended for delivery to the respiratory (e.g. nasal) mucosa, typically it is formulated as an aqueous solution for administration as an aerosol or nasal drops, or alternatively, as a dry powder, e.g. for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

In one embodiment, the herein described subject can be a mammal, preferably a human.

Provided herein, in one aspect are compositions comprising at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 1-5, and a pharmaceutically acceptable buffer or excipient. In some embodiments, the composition comprises at least two polypeptides having an amino acid sequence at least 90% identical to a sequence set forth as SEQ ID NO: 1-5. In some embodiments, the composition comprises at least three polypeptides having an amino acid sequence at least 90% identical to a sequence set forth as SEQ ID NO: 1-5. In some embodiments, the composition comprises at least four polypeptides having an amino acid sequence at least 90% identical to a sequence set forth as SEQ ID NO: 1-5. In some embodiments, the composition comprises the polypeptides having an amino acid sequence at least 90% identical to SEQ ID NO: 1-5. In some embodiments, the composition comprises at least one polypeptide having an amino acid sequence at least 90% identical to a sequence set forth as SEQ ID NO: 15-19. In some embodiments, the composition comprises at least one polypeptide having an amino acid sequence at least 90% identical to a sequence set forth as SEQ ID NO: 20-24. In some embodiments, the composition comprises comprising an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises a pharmaceutically acceptable buffer or excipient. In some embodiments, the composition elicits at least a B cell or a T cell response when administered to a subject.

In another aspect, there are provided compositions comprising at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 6-10, and a pharmaceutically acceptable buffer or excipient. In some embodiments, the composition comprises at least two polypeptides having an amino acid sequence at least 90% identical to a sequence set forth as SEQ ID NO: 6-10. In some embodiments, the composition comprises at least three polypeptides having an amino acid sequence at least 90% identical to a sequence set forth as SEQ ID NO: 6-10. In some embodiments, the composition comprises at least four polypeptides having an amino acid sequence at least 90% identical to a sequence set forth as SEQ ID NO: 6-10. In some embodiments, the composition comprises the polypeptides having an amino acid sequence at least 90% identical to SEQ ID NO: 6-10. In some embodiments, the composition comprises at least one polypeptide having an amino acid sequence at least 90% identical to a sequence set forth as SEQ ID NO: 15-19. In some embodiments, the composition comprises at least one polypeptide having an amino acid sequence at least 90% identical to a sequence set forth as SEQ ID NO: 20-24. In some embodiments, the composition comprises comprising an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises a pharmaceutically acceptable buffer or excipient. In some embodiments, the composition elicits at least a B cell or a T cell response when administered to a subject. In some embodiments, the composition comprises at least one polynucleotide encoding an amino acid sequence at least 90% identical to a sequence set forth as SEQ ID NO: 15-19. In some embodiments, the composition comprises at least one polynucleotide encoding an amino acid sequence at least 90% identical to a sequence set forth as SEQ ID NO: 20-24. In some embodiments, the composition comprises an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises a pharmaceutically acceptable buffer or excipient. In some embodiments, the composition elicits at least a B cell or a T cell response when administered to a subject.

In additional aspects, there are provided compositions comprising at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 1-5, and a pharmaceutically acceptable buffer or excipient. In some embodiments, the composition comprises at least two polynucleotides encoding an amino acid sequence at least 90% identical to a sequence set forth as SEQ ID NO: 1-5. In some embodiments, the composition comprises at least three polynucleotides encoding an amino acid sequence at least 90% identical to a sequence set forth as SEQ ID NO: 1-5. In some embodiments, the composition comprises at least four polynucleotides encoding an amino acid sequence at least 90% identical to a sequence set forth as SEQ ID NO: 1-5. In some embodiments, the composition comprises the polynucleotides encoding an amino acid sequence at least 90% identical to SEQ ID NO: 1-5.

In a further aspect, there are provided compositions comprising at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 6-10, and a pharmaceutically acceptable buffer or excipient. In some embodiments, the composition comprises at least two polynucleotides encoding an amino acid sequence at least 90% identical to a sequence set forth as SEQ ID NO: 6-10. In some embodiments, the composition comprises at least three polynucleotides encoding an amino acid sequence at least 90% identical to a sequence set forth as SEQ ID NO: 6-10. In some embodiments, the composition comprises at least four polynucleotides encoding an amino acid sequence at least 90% identical to a sequence set forth as SEQ ID NO: 6-10. In some embodiments, the composition comprises the polynucleotides encoding an amino acid sequence at least 90% identical to SEQ ID NO: 6-10. In some embodiments, the composition comprises at least one polynucleotide encoding an amino acid sequence at least 90% identical to a sequence set forth as SEQ ID NO: 15-19. In some embodiments, the composition comprises at least one polynucleotide encoding an amino acid sequence at least 90% identical to a sequence set forth as SEQ ID NO: 20-24. In some embodiments, the composition comprises an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises a pharmaceutically acceptable buffer or excipient. In some embodiments, the composition elicits at least a B cell or a T cell response when administered to a subject.

In a further aspect, there are provided compositions comprising (a) at least one polypeptide having an amino acid sequence at least 90% identical to a sequence set forth as SEQ ID NO: 11-14; (b) at least one polypeptide having an amino acid sequence at least 90% identical to a sequence set forth as SEQ ID NO: 15-19 or at least one polypeptide having an amino acid sequence at least 90% identical to a sequence set forth as SEQ ID NO: 20-24; and (c) an effective amount of an adjuvant. In some embodiments, the composition comprises at least two polypeptides having an amino acid sequence at least 90% identical to a sequence set forth as SEQ ID NO: 15-19. In some embodiments, the composition comprises at least three polypeptides having an amino acid sequence at least 90% identical to a sequence set forth as SEQ ID NO: 15-19. In some embodiments, the composition comprises at least four polypeptides having an amino acid sequence at least 90% identical to a sequence set forth as SEQ ID NO: 15-19. In some embodiments, the composition comprises the polypeptides having an amino acid sequence at least 90% identical to SEQ ID NO: 15-19. In some embodiments, the composition comprises at least two polypeptides having an amino acid sequence at least 90% identical to a sequence set forth as SEQ ID NO: 20-24. In some embodiments, the composition comprises at least three polypeptides having an amino acid sequence at least 90% identical to a sequence set forth as SEQ ID NO: 20-24. In some embodiments, the composition comprises at least four polypeptides having an amino acid sequence at least 90% identical to a sequence set forth as SEQ ID NO: 20-24. In some embodiments, the composition comprises the polypeptides having an amino acid sequence at least 90% identical to SEQ ID NO: 20-24. In some embodiments, the composition comprises an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises a pharmaceutically acceptable buffer or excipient. In some embodiments, the composition elicits at least a B cell or a T cell response when administered to a subject.

In another aspect, there are provided compositions comprising (a) at least one polynucleotide encoding an amino acid sequence at least 90% identical to a sequence set forth as SEQ ID NO: 11-14; (b) at least one polynucleotide encoding an amino acid sequence at least 90% identical to a sequence set forth as SEQ ID NO: 15-19 or at least one polynucleotide encoding an amino acid sequence at least 90% identical to a sequence set forth as SEQ ID NO: 20-24; and (c) an effective amount of an adjuvant. In some embodiments, the composition comprises at least two polynucleotides encoding a polypeptide having an amino acid sequence at least 90% identical to a sequence set forth as SEQ ID NO: 15-19. In some embodiments, the composition comprises at least three polynucleotides encoding a polypeptide having an amino acid sequence at least 90% identical to a sequence set forth as SEQ ID NO: 15-19. In some embodiments, the composition comprises at least four polynucleotides encoding a polypeptide having an amino acid sequence at least 90% identical to a sequence set forth as SEQ ID NO: 15-19. In some embodiments, the composition comprises polynucleotides encoding polypeptides having an amino acid sequence at least 90% identical to SEQ ID NO: 15-19. In some embodiments, the composition comprises at least two polynucleotides encoding a polypeptide having an amino acid sequence at least 90% identical to a sequence set forth as SEQ ID NO: 20-24. In some embodiments, the composition comprises at least three polynucleotides encoding a polypeptide having an amino acid sequence at least 90% identical to a sequence set forth as SEQ ID NO: 20-24. In some embodiments, the composition comprises at least four polynucleotides encoding a polypeptide having an amino acid sequence at least 90% identical to a sequence set forth as SEQ ID NO: 20-24. In some embodiments, the composition comprises polynucleotides encoding polypeptides having an amino acid sequence at least 90% identical to SEQ ID NO: 20-24. In some embodiments, the composition comprises an effective amount of an adjuvant. In some embodiments, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some embodiments, the adjuvant comprises CD70. In some embodiments, the adjuvant comprises a nucleic acid encoding a CD70 polypeptide. In some embodiments, the composition comprises comprising a pharmaceutically acceptable buffer or excipient. In some embodiments, the composition elicits at least a B cell or a T cell response when administered to a subject.

In an additional aspect, there are provided compositions comprising (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 1-5; (c) polynucleotides encoding amino acid sequences at least 90% identical to sequences set forth as SEQ ID NO: 15-19; and (d) a polynucleotide encoding a CD70 polypeptide. In a further aspect, there are provided compositions comprising (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 6-10; (c) polynucleotides encoding amino acid sequences at least 90% identical to sequences set forth as SEQ ID NO: 15-19; and (d) a polynucleotide encoding a CD70 polypeptide. In some embodiments, the composition comprises a pharmaceutically acceptable buffer or excipient. In some embodiments, the composition elicits at least a B cell or a T cell response when administered to a subject.

In a further aspect, there are provided methods of eliciting an immune response against a flavivirus in a subject. In some embodiments, the method comprises administering an effective amount of any one of the compositions provided herein. In some embodiments, the flavivirus comprises a dengue virus or a zika virus. In some embodiments, the dengue virus comprises a serotype of a DENV1, DENV2, DENV3, or DENV4. In some embodiments, the immune response comprises at least a B cell or a T cell response. In some embodiments, the method protects the subject from a flavivirus infection. In some embodiments, the method protects the subject from developing severe dengue disease.

In another aspect, there are provided methods of vaccination against flavivirus infection. In some embodiments, the method comprises administering an effective amount of any one of the compositions provided herein. In some embodiments, the flavivirus infection comprises a dengue virus infection or a zika virus infection. In some embodiments, the dengue virus comprises a serotype of a DENV1, DENV2, DENV3, or DENV4. In some embodiments, the method elicits an immune response comprising at least a B cell or a T cell response. In some embodiments, the method protects the subject from developing severe dengue disease.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows the dynamics between AB and T cell responses. Dengvaxia® does not elicit DENV NS protein-specific CD8 T cell responses. A higher incidence of severe dengue was observed among individuals that were DENV-naïve and vaccinated with Dengvaxia® compared with individuals that were DENV-naïve and unvaccinated during subsequent primary DENV infection¹, suggesting that the vaccine primed the naïve individuals for development of severe dengue disease.

FIG. 2 shows an adoptive transfer of DENV-primed T cells can protect mice from antigen-induced ADE. AG129 mice lacking type I and II IFN receptors were left unimmunized or immunized with 10¹¹ genomic equivalents of UV irradiated-DENV2 in alum i.p. on days −14 and −5. One day before infection with 10⁴ PFU of DENV2 (strain S221), mice received 5×10⁷ T cells from AG129 mice infected 7 days prior with S221. Viral RNA levels in the liver quantified on day 3. ***p<0.0001 (from Zellweger et al., Journal of Immunology 2014).

FIG. 3 shows the percentage of survival and morbidity degree of BALB/c mice immunized with pE1D2 and pE2D2 and challenged with DENV2. Mice were immunized intramuscularly (i.m.) with two doses of DNA (100 μg/dose; 2 weeks apart) and then challenged two weeks after the second plasmid injection with DENV2 NGC via an i.e. route. Control groups included mice that were not immunized or injected with pcTPA (control plasmid). Mice were monitored on a daily basis, and (Left) deaths and (Right) clinical signs of infection were recorded. (Left) Differences between pE1D2- and pE2D2 vaccinated animals were statistically significant (p=0.0027), as well as between these groups and control animals (p=0.0001). (Right) The semi-quantitative analysis of morbidity degrees after virus challenge were performed using a subjective scale ranging from 0 to 3 (0=none, 1=mild paralysis in one hind leg or alteration of the spinal column with a small hump, 2=one severe hind leg paralysis and alteration of the spinal column with a small hump or two severe hind leg paralysis, 3=two severe hind leg paralysis and deformed spinal column or death). Asterisks indicate statistically significant differences; *p=0.0051; **p=0.0002; ***p=0.0001. Data represent compilation of two independent experiments, with groups of 10 animals in each test (n=20 mice per group). (from Azevedo et al., PLoS One 2011).

FIG. 4 shows nAb titers in BALB/c mice immunized with pE1D2 and pE2D2. The nAb titers (PRNT₅₀) against DENV2 strain 44-2 was evaluated in serum samples collected from pE1D2- and pE2D2-vaccinated mice (n=20 per group) on 1 day 1 before and 21 days after virus challenge. Mice were immunized twice and challenged with DENV2 NGC two weeks after the last immunization, as described in FIG. 3. Individual samples were serially diluted from 1:5 to 1:640 and standard Vero cell-based PRNT assays were performed in 96-well plates. Asterisks indicate differences that are statistically significant; *p=0.0029; ***p=0.0001. (from Azevedo et al., PLoS One 2011).

FIGS. 5A-B show the protective efficacy of the pE1D2 and YF17D-D2 vaccines in combination. Groups of BALB/c mice (n=10) were immunized with pE1D2 and YF17D-D2 using (FIG. 5A) a prime-boost (i.m. administration of two doses of 100 μg of pE1D2, given two weeks apart, and one subcutaneous (s.c.) injection of the chimeric YF17D-D2 virus (10⁵ PFU) 10 days after the second DNA dose) or (FIG. 5B) simultaneous (injection of a mix of 100 μg of pE1D2 and 10⁵ PFU of YF17D-D2 via i.m. route, designated as Mix) vaccination regimen. Animals were challenged with the DENV2 NGC (4 LD₅₀/4.3 log₁₀; i.c. route) and monitored for 21 days. Morbidity scores were recorded as described in FIG. 3. Asterisks indicate statistically significant differences based on the Mann-Whitney test; *p<0.0⁵, **p<0.01, and ***p<0.001). Id=One dose; 2d=two doses. (from Azevedo et al., PLoS One 2013).

FIGS. 6A-6B show nAb responses against DENV2 in BALB/c mice vaccinated with pE1D2 and YF17D-D2 in combination. FIG. 6A and FIG. 6B refer to samples tested from animals immunized with prime-boost protocols and simultaneous inoculations, respectively. Groups of BALB/c mice were immunized as described in FIGS. 5A-5B, and serum samples were collected 24 h before virus challenge. Sera were serially diluted from 1:5 to 1:640 and the standard Vero cell-based PRNT assay was performed. Asterisks indicate differences that are statistically significant based on the Mann-Whitney test; *p<0.05, **p<0.01, and ***p<0.001. id=One dose; 2d=two doses. (from Azevedo et al., PLoS One 2013).

FIG. 7 shows induction of IFNγ-producing CD8 T cell responses in mice immunized with pE1D2 but not YF17D-D2 vaccine. BALB/c mice were immunized with pE1D2 and YF17D-D2, together or alone, as described in FIG. 5. Spleens were collected from mice (n=5 per group) and processed for IFNγ ELISpot following overnight stimulation with a previously identified H-2d-restricted CD8 T cell epitope (SPCKIPFEI). The number of spot-forming cells (SFC) were quantified. Asterisks indicate statistically significant differences based on the Mann-Whitney test; *p<0.05 and **p<0.01. id=One dose; 2d=two doses. (from Azevedo et al., PLoS One 2013).

FIGS. 8A-8B show the survival and morbidity degree of BALB/c mice immunized with the pcTPANS3, pcTPANS3H and pcTPANS3P DNA vaccine and challenged with DENV2. Mice were immunized with each plasmid twice (100 μg of DNA per dose, given two weeks apart) and challenged two weeks after the second plasmid injection. Non-immunized (naive) animals and mice injected with the control plasmid (pcTPA) received the same virus challenge. Mice were monitored for 21 days. (FIG. 8A) Death and (FIG. 8B) clinical signs of infection were recorded as described in FIG. 3. Asterisks indicate statistically significant differences; *** p<0.0001, ** p<0.01, and * p<0.05. Data represent compilation of two independent experiments, with groups of 10 animals in each test (n=20 mice per group). (from Costa et al., PLoS One 2013).

FIGS. 9A-9D shows a reduced virus-specific CD8 T cell response in Cd27^{-/-} mice with primary DENV2 infection. WT and Cd27^{-/-} mice (4- to 5-week-old; both males and females) were infected with 2×10⁵ focus forming units (FFU) of DENV2 strain JHA1 via an intra-footpad (i.f.) injection route. On day 7 after infection, intracellular cytokine staining (ICS) was performed on splenocytes stimulated with the H-2D^{b} restricted DENV peptide NS4B₉₉₋₁₀₇. CD8 T cells that were (FIG. 9A) CD8α^{low}CD11a⁺ and producing (FIG. 9B) IFNγ, (FIG. 9C) IFNγ and TNF, and (FIG. 9D) granzyme B (Gzm B) were enumerated. n=4-6 mice/group; ***p<0.001; ****p<0.0001.

FIGS. 10A-10D show a reduced virus-specific CD8 T cell response in Cd27^{-/-} mice with primary DENV2 infection. WT and Cd27^{-/-} mice (4- to 5-week-old; both males and females) were infected with 2×10⁵ FFU of DENV2 strain JHA1 via an i.f. route. On day 30 after primary infection, mice were challenged with 2×10⁶ FFU of DENV2 JHA1 via an i.f. route. On day 3 after secondary infection, intracellular cytokine staining (ICS) was performed on splenocytes stimulated with the H-2D^{b} restricted DENV2 peptide NS4B₉₉₋₁₀₇. CD8 T cells that were (FIG. 10A) CD8α^{low}CD11a⁺ and producing (FIG. 10B) IFNγ, (FIG. 10C) IFNγ and TNF, and (FIG. 10D) granzyme B (Gzm B) were enumerated. n=4-6 mice/group; *p<0.05; **p<0.01; ***p<0.001.

FIG. 11 shows the susceptibility of WT and Cd27^{-/-} mice to a lethal DENV2 infection. Four- to 5-week-old male and female WT (n=8) and Cd27^{-/-} mice (n=10) were infected with 2×10⁶ FFU of DENV2 JHA1 (if. route). Mice were monitored daily for survival; p=0.0145, log-rank Mantel-Cox test.

FIG. 12 shows a schematic representation of the DENV genome and DENV1-4 prME expression cassettes. The upper panel shows organization of the RNA genome of DENV. The lower panel shows organization of the DENV1-4 prME expression cassettes inserted into pcDNA3.1 under the CMV promoter. The light grey box indicates the 12-residue signal sequence of DENV capsid sequence. The dark grey box indicates the bovine growth hormone polyadenylation signal (BGHpA).

FIG. 13 shows the detection of antigen-specific CD8 T cells in DENV2-infected mice by tetramer staining. WT C57BL/6 mice that were untreated or injected with 1 mg of Ifnar1-blocking Ab (clone MAR1-5A3; i.p. route; on day 1 before DENV2 infection) were infected with 10⁶ PFU of DENV2 strain S221 via an i.v. route. Controls included uninfected (naïve) mice. Spleens were harvested 7 days after infection and processed for tetramer staining using our previously identified immunodominant epitope NS4B₉₉. A representative FACS plot of each group of 5 mice is shown.

FIGS. 14A-14B show the distribution of DENV1-4 NS3 consensus sequence-derived epitopes that are predicted to bind human MHC class I alleles present in Brazil. In FIG. 14A, the predicted epitopes that are 100% conserved among the four DENV serotypes are located within the helicase domain. Epitopes were predicted using the Immune Epitope Database (IEDB) website tools. FIG. 14B is a graphic representation of the predicted epitopes that are 100% conserved among the four DENV serotypes, and restricted by most frequently represented HLA alleles in Brazil. The cNS3 protein 3D model was generated using the PyMOL program.

FIGS. 15A-15B show fetal weight and size following ZIKV infection of allogeneically pregnant DENV-immune Ifnar1^{-/-} mice with or without depletion of CD8 T cells. DENV2-immune Ifnar1^{-/-} dams were administered isotype control Ab (Isotype) or anti-CD8 Ab (Anti-CD8) and then infected via a retroorbital route at embryonic gestation day 7.5 (E7.5) with 10⁴ FFU of ZIKV SD001 or 10% FBS-PBS as Mock. To generate DENV2-immune mice, mice were inoculated via an intraperitoneal route (i.p.) with 103 FFU of DENV2 S221 for 30 days prior to mating with a BALB/c male. Fetal body weight (FIG. 15A) and size (FIG. 15B) were measured at E14.5. n=38 fetuses from 5 separate mothers (DENV2-immune-Mock), n=8 fetuses from 1 mother (DENV2-immune-ZIKV+isotype), and n=21 fetuses from 3 separate mothers (DENV2-immune-ZIKV+Anti-CD8). Weight and size were determined individually on the residual placenta if fetal resorption was observed. Kruskal-Wallis test; ****P<0.0001.

FIG. 16A-16B show enhanced accumulation of DENV-specific T cells in Ifnar1^{-/-} mice treated with agonist anti-OX40 or anti-4-1BB. Mice (5-week-old males and females) were i.v. infected with 10⁴ FFU of DENV2 S221, followed by i.p. injection of agonist anti-OX40 or anti-4-1BB on day 1. Splenocytes were harvested 7 days p.i. and processed for ICS. FIG. 16A shows numbers of splenic CD8 T cells that were IFNγ+ after stimulation with H-2db restricted peptide NS4B99. FIG. 16B shows numbers of CD4 T cells that were IFNγ+ with I-Ab restricted peptide NS2B108. n=4 mice/group; *P<0.05.

FIG. 17 shows agonist anti-OX40 and anti-4-1BB protect Ifnar1^{-/-} mice from a lethal DENV2 infection. Mice (5-week-old males and females) were i.v. infected with 2×10⁵ FFU of DENV2 S221 and injected with isotype control or agonist anti-OX40 or anti-4-1BB (100 μg i.p.) on day 1 after infection. Survival was assessed over time. n=9 mice/group.

FIG. 18 shows a schematic of T cell and antibody transfer studies.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

Provided herein are compositions and methods for use in eliciting an immune response or vaccinating against a flavivirus, such as a Zika virus, a Dengue virus, or combinations thereof. In some cases, the composition or method provides vaccination against multiple flaviviruses in a single composition.

### Flavivirus Vaccine Compositions

In an aspect, provided herein are compositions comprising a polypeptide comprising a flavivirus B cell epitope, a flavivirus T cell epitope, and a pharmaceutically acceptable buffer or excipient. In some cases, the composition elicits an antibody response and a T cell response against two or more serotypes or species of a flavivirus. In some cases, the composition comprises at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 1-5, and a pharmaceutically acceptable buffer or excipient. In some cases, the composition comprises at least two polypeptides having an amino acid sequence at least 90% identical to a sequence set forth as SEQ ID NO: 1-5. In some cases, the composition comprises at least three polypeptides having an amino acid sequence at least 90% identical to a sequence set forth as SEQ ID NO: 1-5. In some cases, the composition comprises at least four polypeptides having an amino acid sequence at least 90% identical to a sequence set forth as SEQ ID NO: 1-5. In some cases, the composition comprises the polypeptides having an amino acid sequence at least 90% identical to SEQ ID NO: 1-5. In some cases, the composition comprises at least one polypeptide having an amino acid sequence at least 90% identical to a sequence set forth as SEQ ID NO: 15-19. In some cases, the composition comprises at least one polypeptide having an amino acid sequence at least 90% identical to a sequence set forth as SEQ ID NO: 20-24.

In another aspect, there are provided compositions comprising at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 6-10, and a pharmaceutically acceptable buffer or excipient. In some cases, the composition comprises least two polypeptides having an amino acid sequence at least 90% identical to a sequence set forth as SEQ ID NO: 6-10. In some cases, the composition comprises at least three polypeptides having an amino acid sequence at least 90% identical to a sequence set forth as SEQ ID NO: 6-10. In some cases, the composition comprises at least four polypeptides having an amino acid sequence at least 90% identical to a sequence set forth as SEQ ID NO: 6-10. In some cases, the composition comprises the polypeptides having an amino acid sequence at least 90% identical to SEQ ID NO: 6-10. In some cases, the composition comprises at least one polypeptide having an amino acid sequence at least 90% identical to a sequence set forth as SEQ ID NO: 15-19. In some cases, the composition comprises at least one polypeptide having an amino acid sequence at least 90% identical to a sequence set forth as SEQ ID NO: 20-24.

In an additional aspect, there are provided compositions comprising (a) at least one polypeptide having an amino acid sequence at least 90% identical to a sequence set forth as SEQ ID NO: 11-14; (b) at least one polypeptide having an amino acid sequence at least 90% identical to a sequence set forth as SEQ ID NO: 15-19 or at least one polypeptide having an amino acid sequence at least 90% identical to a sequence set forth as SEQ ID NO: 20-24; and (c) an effective amount of an adjuvant. In some cases, the composition comprises at least two polypeptides having an amino acid sequence at least 90% identical to a sequence set forth as SEQ ID NO: 15-19. In some cases, the composition comprises at least three polypeptides having an amino acid sequence at least 90% identical to a sequence set forth as SEQ ID NO: 15-19. In some cases, the composition comprises at least four polypeptides having an amino acid sequence at least 90% identical to a sequence set forth as SEQ ID NO: 15-19. In some cases, the composition comprises the polypeptides having an amino acid sequence at least 90% identical to SEQ ID NO: 15-19. In some cases, the composition comprises at least two polypeptides having an amino acid sequence at least 90% identical to a sequence set forth as SEQ ID NO: 20-24. In some cases, the composition comprises at least three polypeptides having an amino acid sequence at least 90% identical to a sequence set forth as SEQ ID NO: 20-24. In some cases, the composition comprises at least four polypeptides having an amino acid sequence at least 90% identical to a sequence set forth as SEQ ID NO: 20-24. In some cases, the composition comprises the polypeptides having an amino acid sequence at least 90% identical to SEQ ID NO: 20-24.

In aspects of compositions herein, in some cases, the composition comprises at least one, two, three, or four polypeptides having an amino acid sequence at least 90% identical to a sequence set forth as SEQ ID NO: 11-14. In some cases, the composition comprises at least one, two, three, or four polypeptides at least 95% identical to a sequence set forth as SEQ ID NO: 11-14. In some cases, the composition comprises at least one, two, three, or four polypeptides at least 96% identical to a sequence set forth as SEQ ID NO: 11-14. In some cases, the composition comprises at least one, two, three, or four polypeptides at least 97% identical to a sequence set forth as SEQ ID NO: 11-14. In some cases, the composition comprises at least one, two, three, or four polypeptides at least 98% identical to a sequence set forth as SEQ ID NO: 11-14. In some cases, the composition comprises at least one, two, three, or four polypeptides at least 99% identical to a sequence set forth as SEQ ID NO: 11-14. In some cases, the composition comprises at least one, two, three, or four polypeptides 100% identical to a sequence set forth as SEQ ID NO: 11-14.

In aspects of compositions herein, in some cases, the composition comprises at least one, two, three, four, or five polypeptides having an amino acid sequence at least 90% identical to a sequence set forth as SEQ ID NO: 1-5. In some cases, the composition comprises at least one, two, three, four, or five polypeptides at least 95% identical to a sequence set forth as SEQ ID NO: 1-5. In some cases, the composition comprises at least one, two, three, four, or five polypeptides at least 96% identical to a sequence set forth as SEQ ID NO: 1-5. In some cases, the composition comprises at least one, two, three, four, or five polypeptides at least 97% identical to a sequence set forth as SEQ ID NO: 1-5. In some cases, the composition comprises at least one, two, three, four, or five polypeptides at least 98% identical to a sequence set forth as SEQ ID NO: 1-5. In some cases, the composition comprises at least one, two, three, four, or five polypeptides at least 99% identical to a sequence set forth as SEQ ID NO: 1-5. In some cases, the composition comprises at least one, two, three, four, or five polypeptides 100% identical to a sequence set forth as SEQ ID NO: 1-5.

In aspects of compositions herein, in some cases, the composition comprises at least one, two, three, four, or five polypeptides having an amino acid sequence at least 90% identical to a sequence set forth as SEQ ID NO: 6-10. In some cases, the composition comprises at least one, two, three, four, or five polypeptides at least 95% identical to a sequence set forth as SEQ ID NO: 6-10. In some cases, the composition comprises at least one, two, three, four, or five polypeptides at least 96% identical to a sequence set forth as SEQ ID NO: 6-10. In some cases, the composition comprises at least one, two, three, four, or five polypeptides at least 97% identical to a sequence set forth as SEQ ID NO: 6-10. In some cases, the composition comprises at least one, two, three, four, or five polypeptides at least 98% identical to a sequence set forth as SEQ ID NO: 6-10. In some cases, the composition comprises at least one, two, three, four, or five polypeptides at least 99% identical to a sequence set forth as SEQ ID NO: 6-10. In some cases, the composition comprises at least one, two, three, four, or five polypeptides 100% identical to a sequence set forth as SEQ ID NO: 6-10.

In aspects of compositions herein, in some cases, the composition comprises at least one, two, three, four, or five polypeptides having an amino acid sequence at least 90% identical to a sequence set forth as SEQ ID NO: 15-19. In some cases, the composition comprises at least one, two, three, four, or five polypeptides at least 95% identical to a sequence set forth as SEQ ID NO: 15-19. In some cases, the composition comprises at least one, two, three, four, or five polypeptides at least 96% identical to a sequence set forth as SEQ ID NO: 15-19. In some cases, the composition comprises at least one, two, three, four, or five polypeptides at least 97% identical to a sequence set forth as SEQ ID NO: 15-19. In some cases, the composition comprises at least one, two, three, four, or five polypeptides at least 98% identical to a sequence set forth as SEQ ID NO: 15-19. In some cases, the composition comprises at least one, two, three, four, or five polypeptides at least 99% identical to a sequence set forth as SEQ ID NO: 15-19. In some cases, the composition comprises at least one, two, three, four, or five polypeptides 100% identical to a sequence set forth as SEQ ID NO: 15-19.

In aspects of compositions herein, in some cases, the composition comprises at least one, two, three, four, or five polypeptides having an amino acid sequence at least 90% identical to a sequence set forth as SEQ ID NO: 20-24. In some cases, the composition comprises at least one, two, three, four, or five polypeptides at least 95% identical to a sequence set forth as SEQ ID NO: 20-24. In some cases, the composition comprises at least one, two, three, four, or five polypeptides at least 96% identical to a sequence set forth as SEQ ID NO: 20-24. In some cases, the composition comprises at least one, two, three, four, or five polypeptides at least 97% identical to a sequence set forth as SEQ ID NO: 20-24. In some cases, the composition comprises at least one, two, three, four, or five polypeptides at least 98% identical to a sequence set forth as SEQ ID NO: 20-24. In some cases, the composition comprises at least one, two, three, four, or five polypeptides at least 99% identical to a sequence set forth as SEQ ID NO: 20-24. In some cases, the composition comprises at least one, two, three, four, or five polypeptides 100% identical to a sequence set forth as SEQ ID NO: 20-24.

In an aspect, provided herein are compositions comprising a polynucleotide encoding a polypeptide comprising a flavivirus B cell epitope, a flavivirus T cell epitope, and a pharmaceutically acceptable buffer or excipient. In some cases, the composition elicits an antibody response and a T cell response against two or more serotypes or species of a flavivirus. In some cases, the composition comprises at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 1-5, and a pharmaceutically acceptable buffer or excipient. In some cases, the composition comprises at least two polynucleotides encoding an amino acid sequence at least 90% identical to a sequence set forth as SEQ ID NO: 1-5. In some cases, the composition comprises at least three polynucleotides encoding an amino acid sequence at least 90% identical to a sequence set forth as SEQ ID NO: 1-5. In some cases, the composition comprises at least four polynucleotides encoding an amino acid sequence at least 90% identical to a sequence set forth as SEQ ID NO: 1-5. In some cases, the composition comprises the polynucleotides encoding an amino acid sequence at least 90% identical to SEQ ID NO: 1-5. In some cases, the composition comprises at least one polynucleotide encoding an amino acid sequence at least 90% identical to a sequence set forth as SEQ ID NO: 15-19. In some cases, the composition comprises comprising at least one polynucleotide encoding an amino acid sequence at least 90% identical to a sequence set forth as SEQ ID NO: 20-24.

In another aspect, there are provided compositions comprising at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 6-10, and a pharmaceutically acceptable buffer or excipient. In some cases, the composition comprises at least two polynucleotides encoding an amino acid sequence at least 90% identical to a sequence set forth as SEQ ID NO: 6-10. In some cases, the composition comprises at least three polynucleotides encoding an amino acid sequence at least 90% identical to a sequence set forth as SEQ ID NO: 6-10. In some cases, the composition comprises at least four polynucleotides encoding an amino acid sequence at least 90% identical to a sequence set forth as SEQ ID NO: 6-10. In some cases, the composition comprises the polynucleotides encoding an amino acid sequence at least 90% identical to SEQ ID NO: 6-10. In some cases, the composition comprises at least one polynucleotide encoding an amino acid sequence at least 90% identical to a sequence set forth as SEQ ID NO: 15-19. In some cases, the composition comprises comprising at least one polynucleotide encoding an amino acid sequence at least 90% identical to a sequence set forth as SEQ ID NO: 20-24.

In another aspect, there are provided compositions comprising (a) at least one polynucleotide encoding an amino acid sequence at least 90% identical to a sequence set forth as SEQ ID NO: 11-14; (b) at least one polynucleotide encoding an amino acid sequence at least 90% identical to a sequence set forth as SEQ ID NO: 15-19 or at least one polynucleotide encoding an amino acid sequence at least 90% identical to a sequence set forth as SEQ ID NO: 20-24; and (c) an effective amount of an adjuvant. In some cases, the composition comprises at least two polynucleotides encoding a polypeptide having an amino acid sequence at least 90% identical to a sequence set forth as SEQ ID NO: 15-19. In some cases, the composition comprises at least three polynucleotides encoding a polypeptide having an amino acid sequence at least 90% identical to a sequence set forth as SEQ ID NO: 15-19. In some cases, the composition comprises at least four polynucleotides encoding a polypeptide having an amino acid sequence at least 90% identical to a sequence set forth as SEQ ID NO: 15-19. In some cases, the composition comprises polynucleotides encoding polypeptides having an amino acid sequence at least 90% identical to SEQ ID NO: 15-19. In some cases, the composition comprises at least two polynucleotides encoding a polypeptide having an amino acid sequence at least 90% identical to a sequence set forth as SEQ ID NO: 20-24. In some cases, the composition comprises at least three polynucleotides encoding a polypeptide having an amino acid sequence at least 90% identical to a sequence set forth as SEQ ID NO: 20-24. In some cases, the composition comprises at least four polynucleotides encoding a polypeptide having an amino acid sequence at least 90% identical to a sequence set forth as SEQ ID NO: 20-24. In some cases, the composition comprises polynucleotides encoding polypeptides having an amino acid sequence at least 90% identical to SEQ ID NO: 20-24.

In another aspect, there are provided compositions comprising (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 1-5; (c) polynucleotides encoding amino acid sequences at least 90% identical to sequences set forth as SEQ ID NO: 15-19; and (d) a polynucleotide encoding a CD70 polypeptide.

In another aspect, there are provided compositions comprising (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 6-10; (c) polynucleotides encoding amino acid sequences at least 90% identical to sequences set forth as SEQ ID NO: 15-19; and (d) a polynucleotide encoding a CD70 polypeptide.

In aspects of compositions herein, in some cases, the composition comprises at least one, two, three, or four polynucleotides encoding a polypeptide having an amino acid sequence at least 90% identical to a sequence set forth as SEQ ID NO: 11-14. In some cases, the composition comprises at least one, two, three, or four polynucleotides encoding a polypeptide at least 95% identical to a sequence set forth as SEQ ID NO: 11-14. In some cases, the composition comprises at least one, two, three, or four polynucleotides encoding a polypeptide at least 96% identical to a sequence set forth as SEQ ID NO: 11-14. In some cases, the composition comprises at least one, two, three, or four polynucleotides encoding a polypeptide at least 97% identical to a sequence set forth as SEQ ID NO: 11-14. In some cases, the composition comprises at least one, two, three, or four polynucleotides encoding a polypeptide at least 98% identical to a sequence set forth as SEQ ID NO: 11-14. In some cases, the composition comprises at least one, two, three, or four polynucleotides encoding a polypeptide at least 99% identical to a sequence set forth as SEQ ID NO: 11-14. In some cases, the composition comprises at least one, two, three, or four polynucleotides encoding a polypeptide 100% identical to a sequence set forth as SEQ ID NO: 11-14.

In aspects of compositions herein, in some cases, the composition comprises at least one, two, three, four, or five polynucleotides encoding a polypeptide having an amino acid sequence at least 90% identical to a sequence set forth as SEQ ID NO: 1-5. In some cases, the composition comprises at least one, two, three, four, or five polynucleotides encoding a polypeptide at least 95% identical to a sequence set forth as SEQ ID NO: 1-5. In some cases, the composition comprises at least one, two, three, four, or five polynucleotides encoding a polypeptide at least 96% identical to a sequence set forth as SEQ ID NO: 1-5. In some cases, the composition comprises at least one, two, three, four, or five polynucleotides encoding a polypeptide at least 97% identical to a sequence set forth as SEQ ID NO: 1-5. In some cases, the composition comprises at least one, two, three, four, or five polynucleotides encoding a polypeptide at least 98% identical to a sequence set forth as SEQ ID NO: 1-5. In some cases, the composition comprises at least one, two, three, four, or five polynucleotides encoding a polypeptide at least 99% identical to a sequence set forth as SEQ ID NO: 1-5. In some cases, the composition comprises at least one, two, three, four, or five polynucleotides encoding a polypeptide 100% identical to a sequence set forth as SEQ ID NO: 1-5.

In aspects of compositions herein, in some cases, the composition comprises at least one, two, three, four, or five polynucleotides encoding a polypeptide having an amino acid sequence at least 90% identical to a sequence set forth as SEQ ID NO: 6-10. In some cases, the composition comprises at least one, two, three, four, or five polynucleotides encoding a polypeptide at least 95% identical to a sequence set forth as SEQ ID NO: 6-10. In some cases, the composition comprises at least one, two, three, four, or five polynucleotides encoding a polypeptide at least 96% identical to a sequence set forth as SEQ ID NO: 6-10. In some cases, the composition comprises at least one, two, three, four, or five polynucleotides encoding a polypeptide at least 97% identical to a sequence set forth as SEQ ID NO: 6-10. In some cases, the composition comprises at least one, two, three, four, or five polynucleotides encoding a polypeptide at least 98% identical to a sequence set forth as SEQ ID NO: 6-10. In some cases, the composition comprises at least one, two, three, four, or five polynucleotides encoding a polypeptide at least 99% identical to a sequence set forth as SEQ ID NO: 6-10. In some cases, the composition comprises at least one, two, three, four, or five polynucleotides encoding a polypeptide 100% identical to a sequence set forth as SEQ ID NO: 6-10.

In aspects of compositions herein, in some cases, the composition comprises at one, two, three, four, or five polynucleotides encoding a polypeptide having an amino acid sequence at least 90% identical to a sequence set forth as SEQ ID NO: 15-19. In some cases, the composition comprises at least one, two, three, four, or five polynucleotides encoding a polypeptide at least 95% identical to a sequence set forth as SEQ ID NO: 15-19. In some cases, the composition comprises at least one, two, three, four, or five polynucleotides encoding a polypeptide at least 96% identical to a sequence set forth as SEQ ID NO: 15-19. In some cases, the composition comprises at least one, two, three, four, or five polynucleotides encoding a polypeptide at least 97% identical to a sequence set forth as SEQ ID NO: 15-19. In some cases, the composition comprises at least one, two, three, four, or five polynucleotides encoding a polypeptide at least 98% identical to a sequence set forth as SEQ ID NO: 15-19. In some cases, the composition comprises at least one, two, three, four, or five polynucleotides encoding a polypeptide at least 99% identical to a sequence set forth as SEQ ID NO: 15-19. In some cases, the composition comprises at least one, two, three, four, or five polynucleotides encoding a polypeptide 100% identical to a sequence set forth as SEQ ID NO: 15-19.

In aspects of compositions herein, in some cases, the composition comprises at least one, two, three, four, or five polynucleotides encoding a polypeptide having an amino acid sequence at least 90% identical to a sequence set forth as SEQ ID NO: 20-24. In some cases, the composition comprises at least one, two, three, four, or five polynucleotides encoding a polypeptide at least 95% identical to a sequence set forth as SEQ ID NO: 20-24. In some cases, the composition comprises at least one, two, three, four, or five polynucleotides encoding a polypeptide at least 96% identical to a sequence set forth as SEQ ID NO: 20-24. In some cases, the composition comprises at least one, two, three, four, or five polynucleotides encoding a polypeptide at least 97% identical to a sequence set forth as SEQ ID NO: 20-24. In some cases, the composition comprises at least one, two, three, four, or five polynucleotides encoding a polypeptide at least 98% identical to a sequence set forth as SEQ ID NO: 20-24. In some cases, the composition comprises at least one, two, three, four, or five polynucleotides encoding a polypeptide at least 99% identical to a sequence set forth as SEQ ID NO: 20-24. In some cases, the composition comprises at least one, two, three, four, or five polynucleotides encoding a polypeptide 100% identical to a sequence set forth as SEQ ID NO: 20-24.

In aspects of compositions herein, in some cases, the composition comprises a polynucleotide having a sequence at least 90% identical to a sequence set forth as SEQ ID NO: 33, 35, 37, 39, 41, 43, 45, or 47. In some embodiments, the composition comprises at least one, two, three, four, or five, or more polynucleotides having a sequence at least 90% identical to a sequence set forth as SEQ ID NO: 33, 35, 37, 39, 41, 43, 45, or 47. In some embodiments, the composition comprises a polynucleotide having a sequence at least 91, 92, 93, 94, 95, 96, 97, 98, or 99% identical to a sequence set forth as SEQ ID NO: 33, 35, 37, 39, 41, 43, 45, or 47. In some embodiments, the polynucleotide is a messenger ribonucleic acid (mRNA) molecule.

In aspects of compositions herein, in some cases, the composition comprises a polynucleotide encoding an amino acid sequence at least 90% identical to a sequence set forth as SEQ ID NO: 34, 36, 38, 40, 42, 44, 46, or 48. In some embodiments, the composition comprises at least one, two, three, four, or five, or more polynucleotides encoding an amino acid sequence at least 90% identical to a sequence set forth as SEQ ID NO: 34, 36, 38, 40, 42, 44, 46, or 48. In some embodiments, the composition comprises a polynucleotide encoding an amino acid sequence at least 91, 92, 93, 94, 95, 96, 97, 98, or 99% identical to a sequence set forth as SEQ ID NO: 34, 36, 38, 40, 42, 44, 46, or 48. In some embodiments, the polynucleotide is a messenger ribonucleic acid (mRNA) molecule.

In embodiments, of compositions herein, the polypeptide comprises one of a flavivirus envelope, membrane, premembrane, core, NS1, NS2A, NS2B, NS3, NS4A, NS4B, NS5 protein or a subsequence thereof. In some cases, the polypeptide comprises at least two of a flavivirus envelope, membrane, premembrane, core, NS1, NS2A, NS2B, NS3, NS4A, NS4B, NS5 protein or a subsequence thereof. In some cases, the polypeptide comprises at least three of a flavivirus envelope, membrane, premembrane, core, NS1, NS2A, NS2B, NS3, NS4A, NS4B, NS5 protein or a subsequence thereof. In some cases, the polypeptide comprises at least four of a flavivirus envelope, membrane, premembrane, core, NS1, NS2A, NS2B, NS3, NS4A, NS4B, NS5 protein or a subsequence thereof. In some cases, the polypeptide comprises at least five of a flavivirus envelope, membrane, premembrane, core, NS1, NS2A, NS2B, NS3, NS4A, NS4B, NS5 protein or a subsequence thereof. In some cases, the polypeptide comprises at least six of a flavivirus envelope, membrane, premembrane, core, NS1, NS2A, NS2B, NS3, NS4A, NS4B, NS5 protein or a subsequence thereof. In some cases, the polypeptide comprises at least six of a flavivirus envelope, membrane, premembrane, core, NS1, NS2A, NS2B, NS3, NS4A, NS4B, NS5 protein or a subsequence thereof. In some cases, the polypeptide comprises at least seven of a flavivirus envelope, membrane, premembrane, core, NS1, NS2A, NS2B, NS3, NS4A, NS4B, NS5 protein or a subsequence thereof.

In embodiments of compositions herein, in some cases the composition comprises an effective amount of an adjuvant. Suitable adjuvants include, but are not limited to those provided herein. In some cases, the adjuvant increases the immune response to the polypeptide when administered to an individual compared to a polypeptide administered without the adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, Zymosan, or a combination thereof. In some cases, the adjuvant comprises CD70. In some cases the adjuvant comprises a nucleic acid encoding a CD70 polypeptide.

In aspects of compositions herein, in some cases, the composition comprises a pharmaceutically acceptable buffer or excipient. Suitable pharmaceutically acceptable buffers or excipients include, but are not limited to those provided herein. In some cases, the pharmaceutically acceptable buffer or excipient acts to solubilize, stabilize, or preserve the polypeptide or polynucleotide and other components of the composition. In some cases, the pharmaceutically acceptable buffer or excipient comprises maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, surfactant polyoxyethylene-sorbitan monooleate, or combinations thereof. In some cases, solutions or suspensions used for parenteral administration include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Compositions herein are formulated for an appropriate route of administration for achieving the most effective immune response. Suitable routes of administration include but are not limited to intradermal, intramuscular, subcutaneous, intravenous, intra-arterial, oral, sublingual, or a combination thereof.

In aspects of compositions herein, in some cases, the composition elicits an immune response, such as at least a B cell or a T cell response when administered to a subject. In some cases, the composition elicits at least a B cell response against at least one flavivirus when administered to a subject. In some cases, the composition elicits at least a B cell response against at least two flaviviruses when administered to a subject. In some cases, the composition elicits at least a B cell response against at least three flaviviruses when administered to a subject. In some cases, the composition elicits at least a B cell response against at least four flaviviruses when administered to a subject. In some cases, the composition elicits at least a B cell response against at least five flaviviruses when administered to the subject. In some cases, the composition elicits at least a T cell response against at least one flavivirus when administered to a subject. In some cases, the composition elicits at least a T cell response against at least two flaviviruses when administered to a subject. In some cases, the composition elicits at least a T cell response against at least three flaviviruses when administered to a subject. In some cases, the composition elicits at least a T cell response against at least four flaviviruses when administered to a subject. In some cases, the composition elicits at least a T cell response against at least five flaviviruses when administered to the subject.

In aspects of compositions herein, in some cases, the composition elicits an immune response, such as a B cell response or a T cell response against a flavivirus when administered to a subject, wherein the flavivirus comprises one or more of Dengue virus, Zika virus, Yellow Fever virus, West Nile Virus, Japanese encephalitis, tick-borne encephalitis, cell fusing agent virus (CFAV), Palm Creek virus (PCV), or Parramatta River virus (PaRV). In some cases the flavivirus is a Dengue virus serotype 1 (DENV1), Dengue virus serotype 2 (DENV2), Dengue virus serotype 3 (DENV3), or Dengue virus serotype 4 (DENV4).

In another aspect, provided herein are compositions comprising a protein or peptide, or variant, homologue, derivative or subsequence thereof, that comprises, consists, or consists essentially of a flavivirus B cell epitope, or a nucleic acid molecule encoding the protein or peptide, or variant, homologue, derivative or subsequence thereof, and a protein or peptide, or variant, homologue, derivative or subsequence thereof, that comprises, consists or consists essentially of a flavivirus T cell epitope or a nucleic acid molecule encoding the protein or peptide, or variant, homologue, derivative or subsequence thereof. In some cases, the composition elicits, stimulates, induces, promotes, increases, or enhances an antibody response and a T cell response against two or more different serotypes of a flavivirus or two or more different species of flavivirus. In some cases, the composition elicits, stimulates, induces, promotes, increases, or enhances an antibody response against two or more different serotypes of a flavivirus and two or more different species of flavivirus and a T cell response against two or more different serotypes of a flavivirus and two or more different species of flavivirus. In some cases, the protein or peptide, or variant, homologue, derivative, or subsequence thereof, that comprises, consists, or consists essentially of a flavivirus B cell epitope, is a flavivirus envelope, membrane, premembrane, core, NS1, NS2A, NS2B, NS3, NS4A, NS4B or NS5 protein or peptide. In some cases, the protein or peptide, or variant, homologue, derivative, or subsequence thereof, that comprises, consists, or consists essentially of a flavivirus T cell epitope, is a flavivirus envelope, membrane, premembrane, core, NS1, NS2A, NS2B, NS3, NS4A, NS4B or NS5 protein or peptide.

In aspects of compositions provided herein, in some cases, the composition comprises a flavivirus B cell epitope. In some cases, the composition comprises two or more proteins or peptides, or variants, homologues, derivatives or subsequences thereof, from two or more different serotypes of a flavivirus or two or more different species of flavivirus, or nucleic acid molecules encoding two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus B cell epitope. In some cases, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus B cell epitope.

In aspects of compositions provided herein, in some cases, the composition comprises a flavivirus T cell epitope. In some cases, the composition comprises two or more proteins or peptides, or variants, homologues, derivatives or subsequences thereof, from two or more different serotypes of a flavivirus or two or more different species of flavivirus, or nucleic acid molecules encoding the two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus T cell epitope. In some cases, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus T cell epitope.

In aspects of compositions provided herein, in some cases compositions comprise proteins or peptides, or variants, homologues, derivatives or subsequences thereof from DENV 1-4 Dengue virus serotypes, or nucleic acid molecules encoding the proteins or peptides, or variants, homologues, derivatives or subsequences thereof, from DENV 1-4 Dengue virus serotypes. In some cases, compositions comprise proteins or peptides, or variants, homologues, derivatives, or subsequences thereof from five Dengue virus serotypes, or nucleic acid molecules encoding the proteins or peptides, or variants, homologues, derivatives, or subsequences thereof, from five Dengue virus serotypes. In some cases, compositions further comprise a protein, or variant, homologue, derivatives, or subsequence thereof from Zika virus, or nucleic acid molecules encoding the protein, or variant, homologue, derivative, or subsequence thereof, from Zika virus. In some cases, compositions comprise the premembrane and envelope proteins or peptides, or variants, homologues, derivatives, or subsequences thereof, from DENV 1-4 Dengue virus serotypes, or nucleic acid molecules encoding the premembrane and envelope proteins or peptides, or variants, homologues, derivatives, or subsequences thereof, from DENV 1-4 Dengue virus serotypes. In some cases, compositions comprise the premembrane and envelope proteins or peptides, or variants, homologues, derivatives, or subsequences thereof, from five Dengue virus serotypes, or nucleic acid molecules encoding the premembrane and envelope proteins or peptides, or variants, homologues, derivatives, or subsequences thereof, from five Dengue virus serotypes. In some cases, compositions comprise comprising the premembrane and envelope proteins or peptides, or a variants, homologues, derivatives, or subsequences thereof, from Zika virus, or nucleic acid molecules encoding the premembrane and envelope proteins or peptides, or variants, homologues, derivatives, or subsequences thereof, from Zika virus. In some cases, compositions comprise a protein, or variant, homologue, derivative or subsequence thereof, that comprises, consists or consists essentially of a consensus amino acid sequence derived from proteins or peptides from two or more different serotypes of a flavivirus or proteins or peptides from two or more different species of flavivirus, or nucleic acid molecules encoding the consensus amino acid sequence derived from proteins or peptides from two or more different serotypes of a flavivirus or proteins or peptides from two or more different species of flavivirus. In some cases, compositions comprise a protein, or variant, homologue, derivative or subsequence thereof, that comprises, consists or consists essentially of a consensus sequence derived from proteins or peptides from two or more different serotypes of a flavivirus and two or more different species of flavivirus, or nucleic acid molecules encoding the consensus sequence derived from proteins or peptides from two or more different serotypes of a flavivirus and two or more different species of flavivirus. In some cases, compositions comprise a protein, or variant, homologue, derivative or subsequence thereof, that comprises, consists or consists essentially of a consensus sequence derived from the NS3 proteins or peptides from two or more different serotypes of a flavivirus or two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus or two or more different species of flavivirus. In some cases, compositions comprise a protein, or variant, homologue, derivative or subsequence thereof, that comprises, consists or consists essentially of a consensus sequence derived from the NS3 proteins or peptides from two or more different serotypes of a flavivirus and two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus and two or more different species of flavivirus. In some cases, compositions comprise a protein, or variant, homologue, derivative or subsequence thereof, that comprises, consists or consists essentially of a consensus sequence derived from the NS3 proteins or peptides from DENV 1-4 serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from DENV 1-4serotypes of Dengue virus and Zika virus. In some cases, compositions comprise a protein, or variant, homologue, derivative or subsequence thereof, that comprises, consists or consists essentially of a consensus sequence derived from the NS3 proteins or peptides from five serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from five serotypes of Dengue virus and Zika virus.

In aspects of compositions provided herein, in some cases, compositions comprise a protein, or variant, homologue, derivative or subsequence thereof, that comprises, consists, or consists essentially of one or more an amino acid sequence provided in Table 1, or a nucleic acid molecule encoding a protein, or variant, homologue, derivative or subsequence thereof, that comprises, consists, or consists essentially of one or more an amino acid sequence provided in Table 1.

In aspects of compositions provided herein, in some cases the polynucleotide encoding a polypeptide according to any embodiment herein comprises a vector sequence. Vectors and vector sequences herein are contemplated to include one or more promoters or enhancers. In some cases, the vector sequence further comprises an origin of replication for propagating the vector in a microorganism, such as bacteria. In some cases, the vector is a viral vector such as an adenoviral vector or an adeno-associated viral vector. In some cases, the composition comprises one or more vectors configured to direct expression of the protein, or variant, homologue, derivative, or subsequence thereof, that comprises, consists, or consists essentially of a flavivirus B cell epitope, and the protein, or variant, homologue, derivative, or subsequence thereof, that comprises, consists, or consists essentially of a flavivirus T cell epitope according to any embodiment provided herein. In some cases, the vector further comprises a polynucleotide encoding an immunostimulatory polypeptide. In some cases, the immunostimulatory polypeptide comprises CD70. In some cases, the composition comprises a vector configured to direct expression of the CD70 protein.

### Methods and Uses for Flavivirus Vaccination

In another aspect, provided herein are methods of vaccination against a flavivirus infection, eliciting an immune response against a flavivirus, or otherwise protecting an individual against a flavivirus, such as a Dengue virus or a Zika virus. In some cases, methods herein comprise administering an effective amount of a composition provided herein. In some cases, methods herein comprise administration of a T cell epitope and/or a B cell epitope from a Dengue virus polypeptide and/or a Zika virus polypeptide.

In aspects of methods of vaccination provided herein, in some cases, a composition is administered to an individual, wherein the composition comprises a protein, or variant, homologue, derivative or subsequence thereof, that comprises, consists or consists essentially of one or more an amino acid sequence provided in Table 1, or a nucleic acid molecule encoding a protein, or variant, homologue, derivative or subsequence thereof, that comprises, consists or consists essentially of one or more an amino acid sequence provided in Table 1.

In aspects of methods of vaccination provided herein, in some cases, a composition is administered to an individual, wherein the composition comprises a polynucleotide encoding a polypeptide according to any embodiment herein comprises a vector sequence. Vectors and vector sequences herein are contemplated to include one or more promoters or enhancers. In some cases, the vector sequence further comprises an origin of replication for propagating the vector in a microorganism, such as bacteria. In some cases, the vector is a viral vector such as an adenoviral vector or an adeno-associated viral vector. In some cases, the composition comprises one or more vectors configured to direct expression of the protein, or variant, homologue, derivative, or subsequence thereof, that comprises, consists, or consists essentially of a flavivirus B cell epitope, and the protein, or variant, homologue, derivative, or subsequence thereof, that comprises, consists, or consists essentially of a flavivirus T cell epitope according to any embodiment provided herein. In some cases, the vector further comprises a polynucleotide encoding an immunostimulatory polypeptide. In some cases, the immunostimulatory polypeptide comprises CD70. In some cases, the composition comprises a vector configured to direct expression of the CD70 protein.

In aspects of methods of vaccination provided herein, in some embodiments, protect a subject who is vaccinated from developing a flavivirus infection. In some cases, the flavivirus comprises a dengue virus or a zika virus. In some cases, the dengue virus comprises a serotype of a DENV1, DENV2, DENV3, or DENV4. In some cases, the method protects the subject from a flavivirus infection. In some cases, the method protects the subject from developing severe dengue disease.

In aspects of methods of vaccination provided herein, in some cases, an immune response against a flavivirus is elicited., wherein the immune response comprises at least a B cell or a T cell response. In some cases, the method protects the subject from a flavivirus infection. In some cases, the method protects the subject from developing severe dengue disease.

In aspects of methods of vaccination against flavivirus infection provided herein, in some cases, the method comprises administering to a subject at risk of flavivirus infection an effective amount a composition provided herein. In some cases, the flavivirus infection comprises a dengue virus infection or a zika virus infection. In some cases, the dengue virus comprises a serotype of a DENV1, DENV2, DENV3, or DENV4. In some cases, the method elicits an immune response comprising at least a B cell or a T cell response. In some cases, the method protects the subject from developing severe dengue disease.

In aspects of methods provided herein, in some cases the method comprises a method of eliciting, stimulating, inducing, promoting, increasing, or enhancing an immune response against a flavivirus, the method comprising administering a composition provided herein. In some cases, the method elicits, stimulates, induces, promotes, increases, or enhances an immune response against two or more different serotypes of a flavivirus or two or more different species of flavivirus. In some cases, the method elicits, stimulates, induces, promotes, increases, or enhances an immune response against two or more different serotypes of a flavivirus and two or more different species of flavivirus.

In additional methods provided herein, in some cases the method comprises a method of vaccinating against, providing a subject with protection against, or treating a subject for a flavivirus infection, the method comprising administering a composition provided herein. In some cases, the method vaccinates against, provides the subject with protection against or treats a subject for infection with two or more different serotypes of a flavivirus or two or more different species of flavivirus. In some cases, the method vaccinates against, provides the subject with protection against or treats a subject for infection with two or more different serotypes of a flavivirus and two or more different species of flavivirus. In some cases, the method prevents, reduces, or inhibits sensitizing the subject to or occurrence in the subject of severe flavivirus disease or disease upon a secondary or subsequent flavivirus infection or following administration of a composition herein subsequent to a prior flavivirus infection in the subject or prior to administration to the subject of a vaccine against a flavivirus.

In an aspect of methods of vaccination provided herein, in some cases, a composition is administered to an individual, wherein the composition comprises a polypeptide comprising a flavivirus B cell epitope, a flavivirus T cell epitope, and a pharmaceutically acceptable buffer or excipient. In some cases, the composition elicits an antibody response and a T cell response against two or more serotypes or species of a flavivirus. In some cases, the composition comprises at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 1-5, and a pharmaceutically acceptable buffer or excipient. In some cases, the composition comprises at least two polypeptides having an amino acid sequence at least 90% identical to a sequence set forth as SEQ ID NO: 1-5. In some cases, the composition comprises at least three polypeptides having an amino acid sequence at least 90% identical to a sequence set forth as SEQ ID NO: 1-5. In some cases, the composition comprises at least four polypeptides having an amino acid sequence at least 90% identical to a sequence set forth as SEQ ID NO: 1-5. In some cases, the composition comprises the polypeptides having an amino acid sequence at least 90% identical to SEQ ID NO: 1-5. In some cases, the composition comprises at least one polypeptide having an amino acid sequence at least 90% identical to a sequence set forth as SEQ ID NO: 15-19. In some cases, the composition comprises at least one polypeptide having an amino acid sequence at least 90% identical to a sequence set forth as SEQ ID NO: 20-24.

In another aspect of methods of vaccination provided herein, in some cases, a composition is administered to an individual, wherein the composition comprises at least one polypeptide having an amino acid sequence at least 90% identical to a sequence set forth as SEQ ID NO: 11-14, at least one polypeptide having an amino acid sequence at least 90% identical to a sequence set forth as SEQ ID NO: 6-10, and a pharmaceutically acceptable buffer or excipient. In some cases, the composition comprises least two polypeptides having an amino acid sequence at least 90% identical to a sequence set forth as SEQ ID NO: 6-10. In some cases, the composition comprises at least three polypeptides having an amino acid sequence at least 90% identical to a sequence set forth as SEQ ID NO: 6-10. In some cases, the composition comprises at least four polypeptides having an amino acid sequence at least 90% identical to a sequence set forth as SEQ ID NO: 6-10. In some cases, the composition comprises the polypeptides having an amino acid sequence at least 90% identical to SEQ ID NO: 6-10. In some cases, the composition comprises at least one polypeptide having an amino acid sequence at least 90% identical to a sequence set forth as SEQ ID NO: 15-19. In some cases, the composition comprises at least one polypeptide having an amino acid sequence at least 90% identical to a sequence set forth as SEQ ID NO: 20-24.

In an additional aspect of methods of vaccination provided herein, in some cases, a composition is administered to an individual, wherein the composition comprises (a) at least one polypeptide having an amino acid sequence at least 90% identical to a sequence set forth as SEQ ID NO: 11-14; (b) at least one polypeptide having an amino acid sequence at least 90% identical to a sequence set forth as SEQ ID NO: 15-19 or at least one polypeptide having an amino acid sequence at least 90% identical to a sequence set forth as SEQ ID NO: 20-24; and (c) an effective amount of an adjuvant. In some cases, the composition comprises at least two polypeptides having an amino acid sequence at least 90% identical to a sequence set forth as SEQ ID NO: 15-19. In some cases, the composition comprises at least three polypeptides having an amino acid sequence at least 90% identical to a sequence set forth as SEQ ID NO: 15-19. In some cases, the composition comprises at least four polypeptides having an amino acid sequence at least 90% identical to a sequence set forth as SEQ ID NO: 15-19. In some cases, the composition comprises the polypeptides having an amino acid sequence at least 90% identical to SEQ ID NO: 15-19. In some cases, the composition comprises at least two polypeptides having an amino acid sequence at least 90% identical to a sequence set forth as SEQ ID NO: 20-24. In some cases, the composition comprises at least three polypeptides having an amino acid sequence at least 90% identical to a sequence set forth as SEQ ID NO: 20-24. In some cases, the composition comprises at least four polypeptides having an amino acid sequence at least 90% identical to a sequence set forth as SEQ ID NO: 20-24. In some cases, the composition comprises the polypeptides having an amino acid sequence at least 90% identical to SEQ ID NO: 20-24.

In aspects of methods of vaccination provided herein, in some cases, a composition is administered to an individual, wherein the composition comprises at least one, two, three, or four polypeptides having an amino acid sequence at least 90% identical to a sequence set forth as SEQ ID NO: 11-14. In some cases, the composition comprises at least one, two, three, or four polypeptides at least 95% identical to a sequence set forth as SEQ ID NO: 11-14. In some cases, the composition comprises at least one, two, three, or four polypeptides at least 96% identical to a sequence set forth as SEQ ID NO: 11-14. In some cases, the composition comprises at least one, two, three, or four polypeptides at least 97% identical to a sequence set forth as SEQ ID NO: 11-14. In some cases, the composition comprises at least one, two, three, or four polypeptides at least 98% identical to a sequence set forth as SEQ ID NO: 11-14. In some cases, the composition comprises at least one, two, three, or four polypeptides at least 99% identical to a sequence set forth as SEQ ID NO: 11-14. In some cases, the composition comprises at least one, two, three, or four polypeptides 100% identical to a sequence set forth as SEQ ID NO: 11-14.

In aspects of methods of vaccination provided herein, in some cases, a composition is administered to an individual, wherein the composition comprises at least one, two, three, four, or five polypeptides having an amino acid sequence at least 90% identical to a sequence set forth as SEQ ID NO: 1-5. In some cases, the composition comprises at least one, two, three, four, or five polypeptides at least 95% identical to a sequence set forth as SEQ ID NO: 1-5. In some cases, the composition comprises at least one, two, three, four, or five polypeptides at least 96% identical to a sequence set forth as SEQ ID NO: 1-5. In some cases, the composition comprises at least one, two, three, four, or five polypeptides at least 97% identical to a sequence set forth as SEQ ID NO: 1-5. In some cases, the composition comprises at least one, two, three, four, or five polypeptides at least 98% identical to a sequence set forth as SEQ ID NO: 1-5. In some cases, the composition comprises at least one, two, three, four, or five polypeptides at least 99% identical to a sequence set forth as SEQ ID NO: 1-5. In some cases, the composition comprises at least one, two, three, four, or five polypeptides 100% identical to a sequence set forth as SEQ ID NO: 1-5.

In aspects of methods of vaccination provided herein, in some cases, a composition is administered to an individual, wherein the composition comprises at least one, two, three, four, or five polypeptides having an amino acid sequence at least 90% identical to a sequence set forth as SEQ ID NO: 6-10. In some cases, the composition comprises at least one, two, three, four, or five polypeptides at least 95% identical to a sequence set forth as SEQ ID NO: 6-10. In some cases, the composition comprises at least one, two, three, four, or five polypeptides at least 96% identical to a sequence set forth as SEQ ID NO: 6-10. In some cases, the composition comprises at least one, two, three, four, or five polypeptides at least 97% identical to a sequence set forth as SEQ ID NO: 6-10. In some cases, the composition comprises at least one, two, three, four, or five polypeptides at least 98% identical to a sequence set forth as SEQ ID NO: 6-10. In some cases, the composition comprises at least one, two, three, four, or five polypeptides at least 99% identical to a sequence set forth as SEQ ID NO: 6-10. In some cases, the composition comprises at least one, two, three, four, or five polypeptides 100% identical to a sequence set forth as SEQ ID NO: 6-10.

In aspects of methods of vaccination provided herein, in some cases, a composition is administered to an individual, wherein the composition comprises at least one, two, three, four, or five polypeptides having an amino acid sequence at least 90% identical to a sequence set forth as SEQ ID NO: 15-19. In some cases, the composition comprises at least one, two, three, four, or five polypeptides at least 95% identical to a sequence set forth as SEQ ID NO: 15-19. In some cases, the composition comprises at least one, two, three, four, or five polypeptides at least 96% identical to a sequence set forth as SEQ ID NO: 15-19. In some cases, the composition comprises at least one, two, three, four, or five polypeptides at least 97% identical to a sequence set forth as SEQ ID NO: 15-19. In some cases, the composition comprises at least one, two, three, four, or five polypeptides at least 98% identical to a sequence set forth as SEQ ID NO: 15-19. In some cases, the composition comprises at least one, two, three, four, or five polypeptides at least 99% identical to a sequence set forth as SEQ ID NO: 15-19. In some cases, the composition comprises at least one, two, three, four, or five polypeptides 100% identical to a sequence set forth as SEQ ID NO: 15-19.

In aspects of methods of vaccination provided herein, in some cases, a composition is administered to an individual, wherein the composition comprises at least one, two, three, four, or five polypeptides having an amino acid sequence at least 90% identical to a sequence set forth as SEQ ID NO: 20-24. In some cases, the composition comprises at least one, two, three, four, or five polypeptides at least 95% identical to a sequence set forth as SEQ ID NO: 20-24. In some cases, the composition comprises at least one, two, three, four, or five polypeptides at least 96% identical to a sequence set forth as SEQ ID NO: 20-24. In some cases, the composition comprises at least one, two, three, four, or five polypeptides at least 97% identical to a sequence set forth as SEQ ID NO: 20-24. In some cases, the composition comprises at least one, two, three, four, or five polypeptides at least 98% identical to a sequence set forth as SEQ ID NO: 20-24. In some cases, the composition comprises at least one, two, three, four, or five polypeptides at least 99% identical to a sequence set forth as SEQ ID NO: 20-24. In some cases, the composition comprises at least one, two, three, four, or five polypeptides 100% identical to a sequence set forth as SEQ ID NO: 20-24.

In an aspect of methods of vaccination provided herein, in some cases, a composition is administered to an individual, wherein the composition comprises a polynucleotide encoding a polypeptide comprising a flavivirus B cell epitope, a flavivirus T cell epitope, and a pharmaceutically acceptable buffer or excipient. In some cases, the composition elicits an antibody response and a T cell response against two or more serotypes or species of a flavivirus. In some cases, the composition comprises at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 1-5, and a pharmaceutically acceptable buffer or excipient. In some cases, the composition comprises at least two polynucleotides encoding an amino acid sequence at least 90% identical to a sequence set forth as SEQ ID NO: 1-5. In some cases, the composition comprises at least three polynucleotides encoding an amino acid sequence at least 90% identical to a sequence set forth as SEQ ID NO: 1-5. In some cases, the composition comprises at least four polynucleotides encoding an amino acid sequence at least 90% identical to a sequence set forth as SEQ ID NO: 1-5. In some cases, the composition comprises the polynucleotides encoding an amino acid sequence at least 90% identical to SEQ ID NO: 1-5. In some cases, the composition comprises at least one polynucleotide encoding an amino acid sequence at least 90% identical to a sequence set forth as SEQ ID NO: 15-19. In some cases, the composition comprises comprising at least one polynucleotide encoding an amino acid sequence at least 90% identical to a sequence set forth as SEQ ID NO: 20-24.

In another aspect of methods of vaccination provided herein, in some cases, a composition is administered to an individual, wherein the composition comprises at least one polynucleotide encoding an amino acid sequence at least 90% identical to a sequence set forth as SEQ ID NO: 11-14, at least one polynucleotide encoding an amino acid sequence at least 90% identical to a sequence set forth as SEQ ID NO: 6-10, and a pharmaceutically acceptable buffer or excipient. In some cases, the composition comprises at least two polynucleotides encoding an amino acid sequence at least 90% identical to a sequence set forth as SEQ ID NO: 6-10. In some cases, the composition comprises at least three polynucleotides encoding an amino acid sequence at least 90% identical to a sequence set forth as SEQ ID NO: 6-10. In some cases, the composition comprises at least four polynucleotides encoding an amino acid sequence at least 90% identical to a sequence set forth as SEQ ID NO: 6-10. In some cases, the composition comprises the polynucleotides encoding an amino acid sequence at least 90% identical to SEQ ID NO: 6-10. In some cases, the composition comprises at least one polynucleotide encoding an amino acid sequence at least 90% identical to a sequence set forth as SEQ ID NO: 15-19. In some cases, the composition comprises comprising at least one polynucleotide encoding an amino acid sequence at least 90% identical to a sequence set forth as SEQ ID NO: 20-24.

In another aspect of methods of vaccination provided herein, in some cases, a composition is administered to an individual, wherein the composition comprises (a) at least one polynucleotide encoding an amino acid sequence at least 90% identical to a sequence set forth as SEQ ID NO: 11-14; (b) at least one polynucleotide encoding an amino acid sequence at least 90% identical to a sequence set forth as SEQ ID NO: 15-19 or at least one polynucleotide encoding an amino acid sequence at least 90% identical to a sequence set forth as SEQ ID NO: 20-24; and (c) an effective amount of an adjuvant. In some cases, the composition comprises at least two polynucleotides encoding a polypeptide having an amino acid sequence at least 90% identical to a sequence set forth as SEQ ID NO: 15-19. In some cases, the composition comprises at least three polynucleotides encoding a polypeptide having an amino acid sequence at least 90% identical to a sequence set forth as SEQ ID NO: 15-19. In some cases, the composition comprises at least four polynucleotides encoding a polypeptide having an amino acid sequence at least 90% identical to a sequence set forth as SEQ ID NO: 15-19. In some cases, the composition comprises polynucleotides encoding polypeptides having an amino acid sequence at least 90% identical to SEQ ID NO: 15-19. In some cases, the composition comprises at least two polynucleotides encoding a polypeptide having an amino acid sequence at least 90% identical to a sequence set forth as SEQ ID NO: 20-24. In some cases, the composition comprises at least three polynucleotides encoding a polypeptide having an amino acid sequence at least 90% identical to a sequence set forth as SEQ ID NO: 20-24. In some cases, the composition comprises at least four polynucleotides encoding a polypeptide having an amino acid sequence at least 90% identical to a sequence set forth as SEQ ID NO: 20-24. In some cases, the composition comprises polynucleotides encoding polypeptides having an amino acid sequence at least 90% identical to SEQ ID NO: 20-24.

In another aspect of methods of vaccination provided herein, in some cases, a composition is administered to an individual, wherein the composition comprises (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 1-5; (c) polynucleotides encoding amino acid sequences at least 90% identical to sequences set forth as SEQ ID NO: 15-19; and (d) a polynucleotide encoding a CD70 polypeptide.

In another aspect of methods of vaccination provided herein, in some cases, a composition is administered to an individual, wherein the composition comprises (a) at least one polynucleotide encoding an amino acid sequence at least 90% identical to a sequence set forth as SEQ ID NO: 11-14; (b) polynucleotides encoding amino acid sequences at least 90% identical to sequences set forth as SEQ ID NO: 6-10; (c) polynucleotides encoding amino acid sequences at least 90% identical to sequences set forth as SEQ ID NO: 15-19; and (d) a polynucleotide encoding a CD70 polypeptide.

In aspects of methods of vaccination provided herein, in some cases, a composition is administered to an individual, wherein the composition comprises at least one, two, three, or four polynucleotides encoding a polypeptide having an amino acid sequence at least 90% identical to a sequence set forth as SEQ ID NO: 11-14. In some cases, the composition comprises at least one, two, three, or four polynucleotides encoding a polypeptide at least 95% identical to a sequence set forth as SEQ ID NO: 11-14. In some cases, the composition comprises at least one, two, three, or four polynucleotides encoding a polypeptide at least 96% identical to a sequence set forth as SEQ ID NO: 11-14. In some cases, the composition comprises at least one, two, three, or four polynucleotides encoding a polypeptide at least 97% identical to a sequence set forth as SEQ ID NO: 11-14. In some cases, the composition comprises at least one, two, three, or four polynucleotides encoding a polypeptide at least 98% identical to a sequence set forth as SEQ ID NO: 11-14. In some cases, the composition comprises at least one, two, three, or four polynucleotides encoding a polypeptide at least 99% identical to a sequence set forth as SEQ ID NO: 11-14. In some cases, the composition comprises at least one, two, three, or four polynucleotides encoding a polypeptide 100% identical to a sequence set forth as SEQ ID NO: 11-14.

In aspects of methods of vaccination provided herein, in some cases, a composition is administered to an individual, wherein the composition comprises at least one, two, three, four, or five polynucleotides encoding a polypeptide having an amino acid sequence at least 90% identical to a sequence set forth as SEQ ID NO: 1-5. In some cases, the composition comprises at least one, two, three, four, or five polynucleotides encoding a polypeptide at least 95% identical to a sequence set forth as SEQ ID NO: 1-5. In some cases, the composition comprises at least one, two, three, four, or five polynucleotides encoding a polypeptide at least 96% identical to a sequence set forth as SEQ ID NO: 1-5. In some cases, the composition comprises at least one, two, three, four, or five polynucleotides encoding a polypeptide at least 97% identical to a sequence set forth as SEQ ID NO: 1-5. In some cases, the composition comprises at least one, two, three, four, or five polynucleotides encoding a polypeptide at least 98% identical to a sequence set forth as SEQ ID NO: 1-5. In some cases, the composition comprises at least one, two, three, four, or five polynucleotides encoding a polypeptide at least 99% identical to a sequence set forth as SEQ ID NO: 1-5. In some cases, the composition comprises at least one, two, three, four, or five polynucleotides encoding a polypeptide 100% identical to a sequence set forth as SEQ ID NO: 1-5.

In aspects of methods of vaccination provided herein, in some cases, a composition is administered to an individual, wherein the composition comprises at least one, two, three, four, or five polynucleotides encoding a polypeptide having an amino acid sequence at least 90% identical to a sequence set forth as SEQ ID NO: 6-10. In some cases, the composition comprises at least one, two, three, four, or five polynucleotides encoding a polypeptide at least 95% identical to a sequence set forth as SEQ ID NO: 6-10. In some cases, the composition comprises at least one, two, three, four, or five polynucleotides encoding a polypeptide at least 96% identical to a sequence set forth as SEQ ID NO: 6-10. In some cases, the composition comprises at least one, two, three, four, or five polynucleotides encoding a polypeptide at least 97% identical to a sequence set forth as SEQ ID NO: 6-10. In some cases, the composition comprises at least one, two, three, four, or five polynucleotides encoding a polypeptide at least 98% identical to a sequence set forth as SEQ ID NO: 6-10. In some cases, the composition comprises at least one, two, three, four, or five polynucleotides encoding a polypeptide at least 99% identical to a sequence set forth as SEQ ID NO: 6-10. In some cases, the composition comprises at least one, two, three, four, or five polynucleotides encoding a polypeptide 100% identical to a sequence set forth as SEQ ID NO: 6-10.

In aspects of methods of vaccination provided herein, in some cases, a composition is administered to an individual, wherein the composition comprises at one, two, three, four, or five polynucleotides encoding a polypeptide having an amino acid sequence at least 90% identical to a sequence set forth as SEQ ID NO: 15-19. In some cases, the composition comprises at least one, two, three, four, or five polynucleotides encoding a polypeptide at least 95% identical to a sequence set forth as SEQ ID NO: 15-19. In some cases, the composition comprises at least one, two, three, four, or five polynucleotides encoding a polypeptide at least 96% identical to a sequence set forth as SEQ ID NO: 15-19. In some cases, the composition comprises at least one, two, three, four, or five polynucleotides encoding a polypeptide at least 97% identical to a sequence set forth as SEQ ID NO: 15-19. In some cases, the composition comprises at least one, two, three, four, or five polynucleotides encoding a polypeptide at least 98% identical to a sequence set forth as SEQ ID NO: 15-19. In some cases, the composition comprises at least one, two, three, four, or five polynucleotides encoding a polypeptide at least 99% identical to a sequence set forth as SEQ ID NO: 15-19. In some cases, the composition comprises at least one, two, three, four, or five polynucleotides encoding a polypeptide 100% identical to a sequence set forth as SEQ ID NO: 15-19.

In aspects of methods of vaccination provided herein, in some cases, a composition is administered to an individual, wherein the composition comprises at least one, two, three, four, or five polynucleotides encoding a polypeptide having an amino acid sequence at least 90% identical to a sequence set forth as SEQ ID NO: 20-24. In some cases, the composition comprises at least one, two, three, four, or five polynucleotides encoding a polypeptide at least 95% identical to a sequence set forth as SEQ ID NO: 20-24. In some cases, the composition comprises at least one, two, three, four, or five polynucleotides encoding a polypeptide at least 96% identical to a sequence set forth as SEQ ID NO: 20-24. In some cases, the composition comprises at least one, two, three, four, or five polynucleotides encoding a polypeptide at least 97% identical to a sequence set forth as SEQ ID NO: 20-24. In some cases, the composition comprises at least one, two, three, four, or five polynucleotides encoding a polypeptide at least 98% identical to a sequence set forth as SEQ ID NO: 20-24. In some cases, the composition comprises at least one, two, three, four, or five polynucleotides encoding a polypeptide at least 99% identical to a sequence set forth as SEQ ID NO: 20-24. In some cases, the composition comprises at least one, two, three, four, or five polynucleotides encoding a polypeptide 100% identical to a sequence set forth as SEQ ID NO: 20-24.

In aspects of methods of vaccination herein, in some cases, the composition is administered to an individual, wherein the composition comprises a polynucleotide having a sequence at least 90% identical to a sequence set forth as SEQ ID NO: 33, 35, 37, 39, 41, 43, 45, or 47. In some embodiments, the composition comprises at least one, two, three, four, or five, or more polynucleotides having a sequence at least 90% identical to a sequence set forth as SEQ ID NO: 33, 35, 37, 39, 41, 43, 45, or 47. In some embodiments, the composition comprises a polynucleotide having a sequence at least 91, 92, 93, 94, 95, 96, 97, 98, or 99% identical to a sequence set forth as SEQ ID NO: 33, 35, 37, 39, 41, 43, 45, or 47. In some embodiments, the polynucleotide is a messenger ribonucleic acid (mRNA) molecule.

In aspects of methods of vaccination herein, in some cases, the composition is administered to an individual, wherein the composition comprises a polynucleotide encoding an amino acid sequence at least 90% identical to a sequence set forth as SEQ ID NO: 34, 36, 38, 40, 42, 44, 46, or 48. In some embodiments, the composition comprises at least one, two, three, four, or five, or more polynucleotides encoding an amino acid sequence at least 90% identical to a sequence set forth as SEQ ID NO: 34, 36, 38, 40, 42, 44, 46, or 48. In some embodiments, the composition comprises a polynucleotide encoding an amino acid sequence at least 91, 92, 93, 94, 95, 96, 97, 98, or 99% identical to a sequence set forth as SEQ ID NO: 34, 36, 38, 40, 42, 44, 46, or 48. In some embodiments, the polynucleotide is a messenger ribonucleic acid (mRNA) molecule.

In embodiments of methods of vaccination provided herein, in some cases, a composition is administered to an individual, wherein the composition comprises one of a flavivirus envelope, membrane, premembrane, core, NS1, NS2A, NS2B, NS3, NS4A, NS4B, NS5 protein or a subsequence thereof. In some cases, the polypeptide comprises at least two of a flavivirus envelope, membrane, premembrane, core, NS1, NS2A, NS2B, NS3, NS4A, NS4B, NS5 protein or a subsequence thereof. In some cases, the polypeptide comprises at least three of a flavivirus envelope, membrane, premembrane, core, NS1, NS2A, NS2B, NS3, NS4A, NS4B, NS5 protein or a subsequence thereof. In some cases, the polypeptide comprises at least four of a flavivirus envelope, membrane, premembrane, core, NS1, NS2A, NS2B, NS3, NS4A, NS4B, NS5 protein or a subsequence thereof. In some cases, the polypeptide comprises at least five of a flavivirus envelope, membrane, premembrane, core, NS1, NS2A, NS2B, NS3, NS4A, NS4B, NS5 protein or a subsequence thereof. In some cases, the polypeptide comprises at least six of a flavivirus envelope, membrane, premembrane, core, NS1, NS2A, NS2B, NS3, NS4A, NS4B, NS5 protein or a subsequence thereof. In some cases, the polypeptide comprises at least six of a flavivirus envelope, membrane, premembrane, core, NS1, NS2A, NS2B, NS3, NS4A, NS4B, NS5 protein or a subsequence thereof. In some cases, the polypeptide comprises at least seven of a flavivirus envelope, membrane, premembrane, core, NS1, NS2A, NS2B, NS3, NS4A, NS4B, NS5 protein or a subsequence thereof.

In of methods of vaccination provided herein, in some cases, a composition is administered to an individual, wherein the composition comprises an effective amount of an adjuvant. Suitable adjuvants include, but are not limited to those provided herein. In some cases, the adjuvant increases the immune response to the polypeptide when administered to an individual compared to a polypeptide administered without the adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, Zymosan, or a combination thereof. In some cases, the adjuvant comprises CD70. In some cases the adjuvant comprises a nucleic acid encoding a CD70 polypeptide.

In aspects of methods of vaccination provided herein, in some cases, a composition is administered to an individual, wherein the composition comprises a pharmaceutically acceptable buffer or excipient. Suitable pharmaceutically acceptable buffers or excipients include, but are not limited to those provided herein. In some cases, the pharmaceutically acceptable buffer or excipient acts to solubilize, stabilize, or preserve the polypeptide or polynucleotide and other components of the composition. In some cases, the pharmaceutically acceptable buffer or excipient comprises maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, surfactant polyoxyethylene-sorbitan monooleate, or combinations thereof. In some cases, solutions or suspensions used for parenteral administration include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Methods of vaccination herein use an appropriate route of administration for achieving the most effective immune response. Suitable routes of administration include but are not limited to intradermal, intramuscular, subcutaneous, intravenous, intra-arterial, oral, sublingual, or a combination thereof.

In aspects of compositions herein, in some cases, the composition elicits an immune response, such as at least a B cell or a T cell response when administered to a subject. In some cases, the composition elicits at least a B cell response against at least one flavivirus when administered to a subject. In some cases, the composition elicits at least a B cell response against at least two flaviviruses when administered to a subject. In some cases, the composition elicits at least a B cell response against at least three flaviviruses when administered to a subject. In some cases, the composition elicits at least a B cell response against at least four flaviviruses when administered to a subject. In some cases, the composition elicits at least a B cell response against at least five flaviviruses when administered to the subject. In some cases, the composition elicits at least a T cell response against at least one flavivirus when administered to a subject. In some cases, the composition elicits at least a T cell response against at least two flaviviruses when administered to a subject. In some cases, the composition elicits at least a T cell response against at least three flaviviruses when administered to a subject. In some cases, the composition elicits at least a T cell response against at least four flaviviruses when administered to a subject. In some cases, the composition elicits at least a T cell response against at least five flaviviruses when administered to the subject.

In aspects of methods of vaccination provided herein, the method elicits an immune response, such as a B cell response or a T cell response against a flavivirus when administered to a subject, wherein the flavivirus comprises one or more of Dengue virus, Zika virus, Yellow Fever virus, West Nile Virus, Japanese encephalitis, tick-borne encephalitis, cell fusing agent virus (CFAV), Palm Creek virus (PCV), or Parramatta River virus (PaRV). In some cases the flavivirus is a Dengue virus serotype 1 (DENV1), Dengue virus serotype 2 (DENV2), Dengue virus serotype 3 (DENV3), or Dengue virus serotype 4 (DENV4).

In another aspect of methods of vaccination provided herein, in some cases, a composition is administered to an individual, wherein the composition comprises a protein or peptide, or variant, homologue, derivative or subsequence thereof, that comprises, consists, or consists essentially of a flavivirus B cell epitope, or a nucleic acid molecule encoding the protein or peptide, or variant, homologue, derivative or subsequence thereof, and a protein or peptide, or variant, homologue, derivative or subsequence thereof, that comprises, consists or consists essentially of a flavivirus T cell epitope or a nucleic acid molecule encoding the protein or peptide, or variant, homologue, derivative or subsequence thereof. In some cases, the composition elicits, stimulates, induces, promotes, increases, or enhances an antibody response and a T cell response against two or more different serotypes of a flavivirus or two or more different species of flavivirus. In some cases, the composition elicits, stimulates, induces, promotes, increases, or enhances an antibody response against two or more different serotypes of a flavivirus and two or more different species of flavivirus and a T cell response against two or more different serotypes of a flavivirus and two or more different species of flavivirus. In some cases, the protein or peptide, or variant, homologue, derivative, or subsequence thereof, that comprises, consists, or consists essentially of a flavivirus B cell epitope, is a flavivirus envelope, membrane, premembrane, core, NS1, NS2A, NS2B, NS3, NS4A, NS4B or NS5 protein or peptide. In some cases, the protein or peptide, or variant, homologue, derivative, or subsequence thereof, that comprises, consists, or consists essentially of a flavivirus T cell epitope, is a flavivirus envelope, membrane, premembrane, core, NS1, NS2A, NS2B, NS3, NS4A, NS4B or NS5 protein or peptide.

In aspects of compositions provided herein, in some cases, the composition comprises a flavivirus B cell epitope. In some cases, the composition comprises two or more proteins or peptides, or variants, homologues, derivatives or subsequences thereof, from two or more different serotypes of a flavivirus or two or more different species of flavivirus, or nucleic acid molecules encoding two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus B cell epitope. In some cases, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus B cell epitope.

In aspects of methods of vaccination provided herein, in some cases, a composition is administered to an individual, wherein the composition comprises a flavivirus T cell epitope. In some cases, the composition comprises two or more proteins or peptides, or variants, homologues, derivatives or subsequences thereof, from two or more different serotypes of a flavivirus or two or more different species of flavivirus, or nucleic acid molecules encoding the two or more proteins or peptides, or variants, homologues, derivatives or subsequences thereof, from two or more different serotypes of a flavivirus or two or more different species of flavivirus, wherein the two or more proteins or peptides, or variants, homologues, derivatives or subsequences thereof, comprise, consist or consist essentially of a flavivirus T cell epitope. In some cases, the composition comprises two or more proteins or peptides, or variants, homologues, derivatives or subsequences thereof, from the same serotype of a flavivirus or the same species of flavivirus, or nucleic acid molecules encoding the two or more proteins or peptides, or variants, homologues, derivatives or subsequences thereof, from the same serotype of a flavivirus or the same species of flavivirus, wherein the two or more proteins or peptides, or variants, homologues, derivatives or subsequences thereof, comprise, consist or consist essentially of a flavivirus T cell epitope.

In aspects of methods of vaccination provided herein, in some cases, a composition is administered to an individual, wherein the composition comprises proteins or peptides, or variants, homologues, derivatives or subsequences thereof from DENV 1-4 Dengue virus serotypes, or nucleic acid molecules encoding the proteins or peptides, or variants, homologues, derivatives or subsequences thereof, from DENV 1-4 Dengue virus serotypes. In some cases, compositions comprise proteins or peptides, or variants, homologues, derivatives, or subsequences thereof from five Dengue virus serotypes, or nucleic acid molecules encoding the proteins or peptides, or variants, homologues, derivatives, or subsequences thereof, from five Dengue virus serotypes. In some cases, compositions further comprise a protein, or variant, homologue, derivatives, or subsequence thereof from Zika virus, or nucleic acid molecules encoding the protein, or variant, homologue, derivative, or subsequence thereof, from Zika virus. In some cases, compositions comprise the premembrane and envelope proteins or peptides, or variants, homologues, derivatives, or subsequences thereof, from DENV 1-4 Dengue virus serotypes, or nucleic acid molecules encoding the premembrane and envelope proteins or peptides, or variants, homologues, derivatives, or subsequences thereof, from DENV 1-4 Dengue virus serotypes. In some cases, compositions comprise the premembrane and envelope proteins or peptides, or variants, homologues, derivatives, or subsequences thereof, from five Dengue virus serotypes, or nucleic acid molecules encoding the premembrane and envelope proteins or peptides, or variants, homologues, derivatives, or subsequences thereof, from five Dengue virus serotypes. In some cases, compositions comprise comprising the premembrane and envelope proteins or peptides, or a variants, homologues, derivatives, or subsequences thereof, from Zika virus, or nucleic acid molecules encoding the premembrane and envelope proteins or peptides, or variants, homologues, derivatives, or subsequences thereof, from Zika virus. In some cases, compositions comprise a protein, or variant, homologue, derivative or subsequence thereof, that comprises, consists or consists essentially of a consensus amino acid sequence derived from proteins or peptides from two or more different serotypes of a flavivirus or proteins or peptides from two or more different species of flavivirus, or nucleic acid molecules encoding the consensus amino acid sequence derived from proteins or peptides from two or more different serotypes of a flavivirus or proteins or peptides from two or more different species of flavivirus. In some cases, compositions comprise a protein, or variant, homologue, derivative or subsequence thereof, that comprises, consists or consists essentially of a consensus sequence derived from proteins or peptides from two or more different serotypes of a flavivirus and two or more different species of flavivirus, or nucleic acid molecules encoding the consensus sequence derived from proteins or peptides from two or more different serotypes of a flavivirus and two or more different species of flavivirus. In some cases, compositions comprise a protein, or variant, homologue, derivative or subsequence thereof, that comprises, consists or consists essentially of a consensus sequence derived from the NS3 proteins or peptides from two or more different serotypes of a flavivirus or two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus or two or more different species of flavivirus. In some cases, compositions comprise a protein, or variant, homologue, derivative or subsequence thereof, that comprises, consists or consists essentially of a consensus sequence derived from the NS3 proteins or peptides from two or more different serotypes of a flavivirus and two or more different species of flavivirus, or a nucleic acid molecule encoding the protein, or variant, homologue, derivative or subsequence thereof, that comprises, consists or consists essentially of a consensus sequence derived from the NS3 proteins or peptides from two or more different serotypes of a flavivirus and two or more different species of flavivirus. In some cases, compositions comprise a protein, or variant, homologue, derivative or subsequence thereof, that comprises, consists or consists essentially of a consensus sequence derived from the NS3 proteins or peptides from DENV 1-4 serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from DENV 1-4serotypes of Dengue virus and Zika virus. In some cases, compositions comprise a protein, or variant, homologue, derivative or subsequence thereof, that comprises, consists or consists essentially of a consensus sequence derived from the NS3 proteins or peptides from five serotypes of Dengue virus and Zika virus, or a nucleic acid molecule encoding the protein, or variant, homologue, derivative or subsequence thereof, that comprises, consists or consists essentially of a consensus sequence derived from the NS3 proteins or peptides from five serotypes of Dengue virus and Zika virus.

### Methods of Vaccine Formulation

In another aspect, there are provided methods of formulating a vaccine against a flavivirus that will not elicit, stimulate, induce, promote, increase, enhance or sensitize a subject to severe flavivirus disease or infection, the method comprising formulating the vaccine to comprise a composition according to any embodiment provided herein. In some cases, the severe flavivirus disease or infection is severe Dengue disease, severe Dengue infection, severe Zika disease or severe Zika infection.

The present application describes experimental results and line of reasoning which supports the development of more effective flavivirus vaccine and/or treatment approach, than what has been previously described.

In one embodiment, the flavivirus vaccine and/or treatment approach relates to ZIKV.

In one embodiment, the flavivirus vaccine and/or treatment approach relates to DENV.

In one embodiment, the flavivirus vaccine and/or treatment approach relates to ZIKV and DENV.

ZIKV is a positive-sense, single-stranded, enveloped RNA flavivirus that was first isolated in 1947 in Uganda from a sentinel rhesus macaque, and until recently was known to cause mild, self-limiting, and sporadic disease in Africa and Southeast Asia.

DENV contains about 11,000 nucleotide bases, which code for the three different types of protein molecules (C, prM, and E) that form the virus particle and seven other types of protein molecules (NS1, NS2a, NS2b, NS3, NS4a, NS4b, NS5) that are found in infected host cells only and are required for replication of the virus. There are five strains of the virus (i.e., serotypes), where the distinctions between the serotypes are based on their antigenicity.

CD8⁺ cytotoxic T cells play a key role in the defense against intracellular pathogens and tumor cells. CD8⁺ T cell immune responses are driven by the recognition of foreign peptides presented by major histocompatibility complex class I (MHC I) molecules at the cell surface. The identification of these peptides (CD8⁺ T cell epitopes) is therefore important for understanding disease pathogenesis and etiology as well as for vaccine design.

A large body of literature has provided evidence for a potential dual role for CD8⁺ T cells in protection and pathogenesis during dengue virus (DENV) infection (Screaton et al., 2015; Tang et al., 2015; Weiskopf and Sette, 2014; Zellweger and Shresta, 2014). Epidemiologic studies indicate that Severe Dengue is most often seen in individuals experiencing a heterotypic DENV infection after prior seroconversion to at least one of the other three serotypes (Guzman et al., 2000; Sangkawibha et al., 1984). Some studies showed cross-reactive CD8⁺ T cells are more activated during secondary infection (Mongkolsapaya et al., 2003) with a suboptimal T cell phenotype (Mongkolsapaya et al., 2006) (Imrie et al., 2007; Mangada and Rothman, 2005) suggesting a possible pathogenic role for cross-reactive T cells. However, recently emerging literature points to a protective role for T cells in DENV infection (Weiskopf et al., 2013; Weiskopf et al., 2015), and our previous work on DENV using mouse models (Prestwood et al., 2012b; Yauch et al., 2010; Yauch et al., 2009; Zellweger et al., 2014; Zellweger et al., 2013; Zellweger et al., 2015) in C57BL/6 and 129/Sv mice lacking type I IFN receptor (IFNAR) alone or both type I and II IFN receptors (AB6, A129, and AG129) has provided multiple lines of evidence indicating a protective role for CD8⁺ T cells. In addition to the role of CD8⁺ T cells, CD4⁺ T cells play a role in eliciting a protective immune response in a flavivirus vaccination setting.

Signs of clinical Zika disease have historically been similar to signs of dengue fever, and ZIKV's immunologic similarity to DENV has also been documented. Blast search results show that ZIKV and DENV have about 52%-57% amino acid sequence homology. Indeed serologic cross-reactivity of these two viruses has probably contributed to misdiagnosis and under-diagnosis of ZIKV, and cases of concurrent infection with ZIKV and DENV have also been documented. Cellular immunity to flaviviruses is also cross-reactive, and cross-reactive T cells can play a dual role in protection and pathogenesis.

Epidemiologic and laboratory studies from the relatively large body of knowledge on the serotypes of DENV indicate that the severe and potentially fatal form of dengue disease occurs most commonly when patients are infected with a second DENV serotype after infection by and recovery from a first heterologous DENV serotype. One hypothesis deemed “original T cell antigenic sin” suggests that disease severity increases in secondary infection because T cells primed during the first DENV infection predominate in the subsequent infection with a different DENV serotype, and these serotype-cross-reactive T cells fail to mount an appropriate immune response to the second DENV serotype. In certain cases, similar T cell cross-reactivity may exist between ZIKV and DENV, as ZIKV and DENV share high amino acid identity. Consistent with this homology, several recent studies have revealed cross-reactivity between ZIKV and DENV at the antibody response level. In particular, both plasma and monoclonal antibodies isolated from DENV-exposed donors can have potent neutralizing activity against ZIKV and can mediate antibody-dependent enhancement (ADE) of ZIKV infection. In fact, monoclonal antibodies isolated from ZIKV-immune donors can induce ADE of DENV infection in vitro and in vivo in mice.

Very little is known, however, about T cell-mediated responses to ZIKV at present. As ZIKV and DENV will continue to co-circulate in many regions of the world due to their common vectors and geographical distributions, it is critical to start exploring the protective vs. potentially pathogenic influence of T cells induced by prior DENV exposure on ZIKV infection.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

“Administering” an expression vector, nucleic acid molecule, or a delivery vehicle (such as a chitosan nanoparticle) to a cell comprises transducing, transfecting, electroporation, translocating, fusing, phagocytosing, shooting or ballistic methods, etc., i.e., any means by which a protein or nucleic acid can be transported across a cell membrane and preferably into the nucleus of a cell.

The term “recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide. The nucleotide sequences are displayed herein in the conventional 5′-3′ orientation.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. The expression “amino acid analogs” refers to compounds that have the same basic chemical structure as a naturally occurring amino acid, i.e., an alpha. carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions, or additions that alter, add, or delete a single amino acid or -nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W.H. Freeman, New York, pages 6-20, for a discussion of amino acid properties):

Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y),
Tryptophan (W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in Table 1 without undue effort.

TABLE 1

| Peptide Sequence | SEQ ID NO: |
|---|---|
| KTVWFVPSIK | 25 |
| TTDISEMGA | 26 |
| FVVTTDISEM | 27 |
| AARGYISTR | 28 |
| RTLILAPTR | 29 |
| LMRRGDLPV | 30 |
| EAKMLLDNI | 31 |
| EIVDLMCHA | 32 |

“Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the present invention refer to their use for amplification of a target nucleic acid sequence, e.g., by the polymerase chain reaction (PCR) or other conventional nucleic-acid amplification methods, such as qPCR.

The phrases “coding sequence,” “structural sequence,” and “structural nucleic acid sequence” refer to a physical structure comprising an orderly arrangement of nucleic acids. The nucleic acids are arranged in a series of nucleic acid triplets that each form a codon. Each codon encodes for a specific amino acid. Thus, the coding sequence, structural sequence, and structural nucleic acid sequence encode a series of amino acids forming a protein, polypeptide, or peptide sequence. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The term “expression” refers to the transcription of a gene to produce the corresponding mRNA and translation of this mRNA to produce the corresponding gene product (i.e., a peptide, polypeptide, or protein).

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

The term “treating” or “treatment” refers to a process by which an infection or a disease or the symptoms of an infection or a disease associated with a flavivirus strain are prevented, alleviated, or completely eliminated. As used herein, the term “prevented” or “preventing” refers to a process by which an infection or a disease or symptoms of an infection or a disease associated with a flavivirus are obstructed or delayed.

In accordance with the invention, treatment methods are provided that include therapeutic (following infection) and prophylactic (prior to flavivirus exposure, infection, or pathology) methods. For example, therapeutic and prophylactic methods of treating a subject for a flavivirus infection include treatment of a subject having or at risk of having a flavivirus infection or pathology, treating a subject with a flavivirus infection, and methods of protecting a subject from a flavivirus infection (e.g., provide the subject with protection against flavivirus infection), to decrease or reduce the probability of a flavivirus infection in a subject, to decrease or reduce susceptibility of a subject to a flavivirus infection, or to inhibit or prevent a flavivirus infection in a subject, and to decrease, reduce, inhibit or suppress transmission of the flavivirus from a host (e.g., a mosquito) to a subject.

Such methods include administering flavivirus protein, peptide, or a variant, modification, homologue, derivative, or subsequence thereof to therapeutically or prophylactically treat (vaccinate or immunize) a subject having or at risk of having a flavivirus infection or pathology. Accordingly, methods can treat the flavivirus infection or pathology, or provide the subject with protection from infection (e.g., prophylactic protection).

In one embodiment, a method includes administering to a subject an amount of flavivirus protein, peptide, or a variant, modification, homologue, derivative, or subsequence thereof sufficient to treat the subject for the flavivirus infection or pathology. In another embodiment, a method includes administering to a subject an amount of a flavivirus B cell epitope and/or T cell epitope sufficient to provide the subject with protection against the flavivirus infection or pathology, or one or more physiological conditions, disorders, illness, diseases, or symptoms caused by or associated with the virus infection or pathology. In a further embodiment, a method includes administering a subject an amount of a flavivirus B cell epitope and/or T cell epitope sufficient to treat the subject for the flavivirus infection.

flavivirus proteins, peptides, or a variant, modification, homologue, derivative, or subsequence thereof to include B cell epitopes and/or T cell epitopes. In one embodiment, a method includes administering an amount of flavivirus protein, peptide, or a variant, modification, homologue, derivative, or subsequence thereof (e.g., a B cell and/or T cell epitope) to a subject in need thereof, sufficient to provide the subject with protection against flavivirus infection or pathology. In another embodiment, a method includes administering an amount of a flavivirus protein, peptide, or a variant, modification, homologue, derivative, or subsequence thereof (e.g., a B cell epitope and/or T cell epitope) to a subject in need thereof sufficient to treat, vaccinate or immunize the subject against the flavivirus infection or pathology.

In accordance with the invention, methods of inducing, increasing, promoting, or stimulating anti-flavivirus activity of CD8⁺ T cells or CD4⁺ T cells in a subject are provided. In one embodiment, a method includes administering to a subject an amount of a flavivirus T cell epitope sufficient to induce, increase, promote or stimulate anti-flavivirus activity of CD8⁺ T cells or CD4⁺ T cells in the subject.

In accordance with the invention, methods of inducing, increasing, promoting, or stimulating anti-flavivirus activity of B cells in a subject are provided. In one embodiment, a method includes administering to a subject an amount of a flavivirus B cell epitope sufficient to induce, increase, promote or stimulate anti-flavivirus activity of B cells in the subject.

In methods of the invention, any appropriate flavivirus protein, peptide, or a variant, modification, homologue, derivative, or subsequence thereof can be administered. Non-limiting examples include flavivirus peptide, subsequence, portion, or modification thereof of a ZIKV or DENV1, DENV2, DENV3, DENV4 or DENV5 serotype. Additional non-limiting examples include a Dengue or Zika virus structural protein (e.g., C, M or E) or non-structural (NS) protein (e.g., NS1, NS2A, NS2B, NS3, NS4A, NS4B or NS5) T cell epitope, such as a subsequence, portion, or modification of a sequence in such structural and non-structural (NS) proteins. Particular non-limiting examples include a peptide sequence set forth in Tables 1, a subsequence thereof or a modification thereof.

In particular methods embodiments, one or more disorders, diseases, physiological conditions, pathologies, and symptoms associated with or caused by a flavivirus infection or pathology will respond to treatment. In particular methods embodiments, treatment methods reduce, decrease, suppress, limit, control or inhibit flavivirus numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a flavivirus nucleic acid. In additional particular methods embodiments, treatment methods include an amount of a flavivirus peptide, subsequence or portion thereof sufficient to increase, induce, enhance, augment, promote or stimulate an immune response against a flavivirus; increase, induce, enhance, augment, promote or stimulate flavivirus clearance or removal; or decrease, reduce, inhibit, suppress, prevent, control, or limit transmission of flavivirus to a subject (e.g., transmission from a host, such as a mosquito, to a subject). In further particular methods embodiments, treatment methods include an amount of flavivirus peptide, subsequence, or portion thereof sufficient to protect a subject from a flavivirus infection or pathology, or reduce, decrease, limit, control or inhibit susceptibility to flavivirus infection or pathology.

Methods of the invention include treatment methods, which result in any therapeutic or beneficial effect. In various methods embodiments, flavivirus infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with flavivirus infection, proliferation or replication, or pathology (e.g., fever, rash, headache, pain behind the eyes, muscle or joint pain, nausea, vomiting, loss of appetite). In additional various particular embodiments, treatment methods include reducing, decreasing, inhibiting, delaying, or preventing onset, progression, frequency, duration, severity, probability or susceptibility of one or more adverse symptoms, disorders, illnesses, diseases or complications caused by or associated with flavivirus infection, proliferation or replication, or pathology (e.g., fever, rash, headache, pain behind the eyes, muscle or joint pain, nausea, vomiting, loss of appetite). In further various particular embodiments, treatment methods include improving, accelerating, facilitating, enhancing, augmenting, or hastening recovery of a subject from a flavivirus infection or pathogenesis, or one or more adverse symptoms, disorders, illnesses, diseases or complications caused by or associated with flavivirus infection, proliferation or replication, or pathology (e.g., fever, rash, headache, pain behind the eyes, muscle or joint pain, nausea, vomiting, loss of appetite). In yet additional various embodiments, treatment methods include stabilizing infection, proliferation, replication, pathogenesis, or an adverse symptom, disorder, illness, disease, or complication caused by or associated with flavivirus infection, proliferation or replication, or pathology, or decreasing, reducing, inhibiting, suppressing, limiting, or controlling transmission of flavivirus from a host (e.g., mosquito) to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease, or complication caused by or associated with flavivirus infection, proliferation or replication, or pathology (e.g., fever, rash, headache, pain behind the eyes, muscle or joint pain, nausea, vomiting, loss of appetite). Thus, a satisfactory clinical endpoint is achieved when there is an incremental improvement or a partial reduction in an adverse symptom, disorder, illness, disease or complication caused by or associated with flavivirus infection, proliferation or replication, or pathology, or an inhibition, decrease, reduction, suppression, prevention, limit or control of worsening or progression of one or more adverse symptoms, disorders, illnesses, diseases or complications caused by or associated with flavivirus infection, flavivirus numbers, titers, proliferation or replication, flavivirus protein or nucleic acid, or flavivirus pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a flavivirus infection or pathology. For example, reducing an amount of an adjunct therapy, for example, a reduction or decrease of a treatment for a flavivirus infection or pathology, or a vaccination or immunization protocol is considered a beneficial effect. In addition, reducing or decreasing an amount of a flavivirus antigen used for vaccination or immunization of a subject to provide protection to the subject is considered a beneficial effect.

Adverse symptoms and complications associated with flavivirus infection and pathology include, for example, e.g., fever, rash, headache, pain behind the eyes, muscle or joint pain, nausea, vomiting, loss of appetite, etc. Other symptoms of flavivirus infection or pathogenesis are known to one of skill in the art and treatment thereof in accordance with the invention is provided. Thus, the aforementioned symptoms and complications are treatable in accordance with the invention.

Methods and compositions of the invention also include increasing, stimulating, promoting, enhancing, inducing, or augmenting an anti-DENV and/or anti-ZIKV B cell, CD4⁺ and/or CD8⁺ T cell responses in a subject, such as a subject with or at risk of a Dengue virus or Zika virus infection or pathology. In one embodiment, a method includes administering to a subject an amount of flavivirus protein, peptide, or a variant, modification, homologue, derivative, or subsequence thereof sufficient to increase, stimulate, promote, enhance, augment, or induce anti-DENV and/or anti-ZIKV B cell, CD4⁺ and/or CD8⁺ T cell response in the subject. In another embodiment, a method includes administering to a subject an amount of flavivirus protein, peptide, or a variant, modification, homologue, derivative or subsequence thereof and administering a flavivirus antigen, live or attenuated flavivirus, or a nucleic acid encoding all or a portion (e.g., a B cell or T cell epitope) of any protein or proteinaceous flavivirus antigen sufficient to increase, stimulate, promote, enhance, augment or induce anti-flavivirus B cell, CD4⁺ T cell or CD8⁺ T cell response in the subject.

Methods of the invention additionally include, among other things, increasing production of a Th1 cytokine (e.g., IFN-gamma, TNF-alpha, IL-1alpha, IL-2, IL-6, IL-8, etc.) or other signaling molecule (e.g., CD40L) in vitro or in vivo. In one embodiment, a method includes administering to a subject in need thereof an amount of flavivirus protein, peptide, or a variant, modification, homologue, derivative, or subsequence thereof sufficient to increase production of a Th1 cytokine in the subject (e.g., IFN-gamma, TNF-alpha, IL-1alpha, IL-2, IL-6, IL-8, etc.) or other signaling molecule (e.g., CD40L).

Methods, uses and compositions of the invention include administration of flavivirus, protein, peptide, or a variant, modification, homologue, derivative or subsequence thereof to a subject prior to contact, exposure or infection by a flavivirus (e.g. Dengue virus or Zika virus), administration prior to, substantially contemporaneously with or after a subject has been contacted by, exposed to or infected with a flavivirus (e.g. Dengue virus or Zika virus), and administration prior to, substantially contemporaneously with or after flavivirus (e.g. Dengue virus or Zika virus) pathology or development of one or more adverse symptoms, disorders, illness or diseases caused by or associated with a flavivirus infection, or pathology. A subject infected with a flavivirus may have an infection over a period of 1-5, 5-10, 10-20, 20-30, 30-50, 50-100 hours, days, months, or years.

Invention compositions (e.g., flavivirus protein peptide, or a variant, modification, homologue, derivative, or subsequence thereof, including B cell epitopes and T cell epitopes) and uses and methods can be combined with any compound, agent, drug, treatment or other therapeutic regimen or protocol having a desired therapeutic, beneficial, additive, synergistic or complementary activity or effect. Exemplary combination compositions and treatments include multiple T cell epitopes as set for the herein, second actives, such as anti-flavivirus compounds, agents, and drugs, as well as agents that assist, promote, stimulate, or enhance efficacy. Such anti-flavivirus drugs, agents, treatments, and therapies can be administered or performed prior to, substantially contemporaneously with or following any other method of the invention, for example, a therapeutic method of treating a subject for a flavivirus infection or pathology, or a method of prophylactic treatment of a subject for a flavivirus infection.

flavivirus proteins, peptides, or variants, modifications, homologues, derivatives, or subsequences thereof can be administered as a combination composition, or administered separately, such as concurrently or in series or sequentially (prior to or following) administering a second active, to a subject. The invention therefore provides combinations in which a method or use of the invention is used in a combination with any compound, agent, drug, therapeutic regimen, treatment protocol, process, remedy, or composition, such as an anti-viral (e.g., flavivirus) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of one or more flavivirus proteins, peptides, or variants, modifications, homologues, derivatives or subsequences thereof, or a nucleic acid encoding all or a portion (e.g., a B cell or T cell epitope) of a flavivirus protein, peptide, or a variant, modification, homologue, derivative or subsequence thereof, to a subject. Specific non-limiting examples of combination embodiments therefore include the foregoing or other compound, agent, drug, therapeutic regimen, treatment protocol, process, remedy, or composition.

An exemplary combination is a flavivirus protein, peptide, variant, modification, homologue, derivative, or subsequence thereof (e.g., a B cell, CD4⁺ T cell, or CD8⁺ T cell epitope) and a different flavivirus protein, peptide, variant, modification, homologue, derivative, or subsequence thereof (e.g., a different B or T cell epitope) such as a B cell epitope, T cell epitope, antigen (e.g., flavivirus extract), or live or attenuated flavivirus (e.g., inactivated flavivirus). Such flavivirus antigens and epitopes set forth herein or known to one skilled in the art include a flavivirus antigen that increases, stimulates, enhances, promotes, augments or induces a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c cells), an anti-flavivirus B cell, CD4⁺ T cell or CD8⁺ T cell response, production of a Th1 cytokine, a T cell mediated immune response, a B cell mediated immune response etc.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

In certain instances, as will be apparent to a person of skill in the art, references to a flavivirus protein, peptide, or a variant, modification, homologue, derivative, or subsequence thereof as used herein also encompasses a nucleic acid molecule encoding the flavivirus protein, peptide, or the variant, modification, homologue, derivative, or subsequence thereof. For example, descriptions methods and composition of the present invention comprising administration of a flavivirus protein, peptide, or a variant, modification, homologue, derivative, or subsequence thereof encompasses administration of a nucleic acid molecule encoding the flavivirus protein, peptide, or the variant, modification, homologue, derivative, or subsequence thereof.

Methods of the invention also include, among other things, methods that result in a reduced need or use of another compound, agent, drug, therapeutic regimen, treatment protocol, process, or remedy. For example, for a flavivirus infection or pathology, vaccination or immunization, a method of the invention has a therapeutic benefit if in a given subject a less frequent or reduced dose or elimination of an anti-flavivirus treatment results. Thus, in accordance with the invention, methods of reducing need or use of a treatment or therapy for a flavivirus infection or pathology, or vaccination or immunization, are provided.

In invention methods in which there is a desired outcome, such as a therapeutic or prophylactic method that provides a benefit from treatment, vaccination, or immunization flavivirus protein, peptide, or a variant, modification, homologue, derivative, or subsequence thereof can be administered in a sufficient or effective amount. As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by administration of a flavivirus protein, peptide, or a variant, modification, homologue, derivative, or subsequence thereof alone or in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment, or therapeutic regimens may be included in order to provide a given subject with a detectable or measurable improvement or benefit to the subject. For example, to increase, enhance, improve, or optimize immunization and/or vaccination, after an initial or primary administration of one or more flavivirus proteins peptides, or variants, modifications, homologues, derivatives, or subsequences thereof to a subject, the subject can be administered one or more additional “boosters” of one or more flavivirus peptides, subsequences, portions, or modifications thereof. Such subsequent “booster” administrations can be of the same or a different formulation, dose or concentration, route, etc.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

The expression “an acceptable carrier” may refer to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

The expression “ELISPOT” refers to the known Enzyme-Linked ImmunoSpot assay which typically allows visualization of the secretory product(s) of individual activated or responding cells. Each spot that develops in the assay represents a single reactive cell. Thus, the ELISPOT assay provides both qualitative (regarding the specific cytokine or other secreted immune molecule) and quantitative (the frequency of responding cells within the test population) information. Generally speaking, in an ELISPOT assay, the membrane surfaces in a 96-well PVDF-membrane microtiter plate are coated with capture antibody that binds a specific epitope of the cytokine being assayed. During the cell incubation and stimulation step, a biological sample (typically containing PBMCs) is seeded into the wells of the plate along with the antigen (which can be a peptide as described in the present disclosure), and forms a monolayer on the membrane surface of the well. As the antigen-specific cells are activated, they release the cytokine, which is captured directly on the membrane surface by the immobilized antibody. The cytokine is thus “captured” in the area directly surrounding the secreting cell, before it has a chance to diffuse into the culture media, or to be degraded by proteases and bound by receptors on bystander cells. Subsequent detection steps visualize the immobilized cytokine as an ImmunoSpot; essentially the secretory footprint of the activated cell.

The terms “determining,” “measuring,” “evaluating,” “assessing,” and “assaying,” as used herein, generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

The expression “biological sample” includes in the present disclosure any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

The expression “treatment” includes inducing, enhancing, or sustaining an immune response against a flavivirus infection or symptoms associated thereto. For example, the treatment may induce, increase, promote or stimulate anti-flavivirus activity of immune system cells in a subject following the treatment. For example, the immune system cells may include T cells, including CD4⁺ T cells, CD8⁺ T cells, and/or B cells.

The expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

TABLE 2
Protein Sequences for Vaccines

| | | | SEQ |
|---|---|---|---|
| Protein | | Accession | ID |
| Name | PROTEIN SEQUENCE | Number | NO: |
| E | MRCVGIGNRDFVEGLSGATWVDVVLEHGSCVTTMAKNKPTL | MG721060 | 1 |
| w/fusion | DIELLKTEVTNPAVLRKLCIEAKISNTTTDSRCPREGEATLVEE | | |
| loop | QDANFVCRRTFVDRGRGNGCGRFGKGSLLTCAKFKCVTKLE | | |
| mutations- | GKIVQYENLKYSVIVTVHTGDQHQVGNETTEHGTIATITPQAP | | |
| DENV1 | TSEIQLTDYGALTLDCSPRTGLDFNEMVLLTMKEKSWLVHKQ | | |
| | WFLDLPLPWTSGASTSQETWNRQDLLVTFKTAHAKKQEVVV | | |
| | LGSQEGAMHTALTGATEIQTSGTTTIFAGHLKCRLKMDKLTL | | |
| | KGVSYVMCTGSFKLEKEVAETQHGTVLVQVKYEGTDAPCKIP | | |
| | ISTQDEKGVTQNGRLITANPIVTDKEKPVNIETEPPFGESYIVIG | | |
| | AGEKALKLSWFKKGSSIGKMFEATARGARRMAILGDTAWDF | | |
| | GSIGGVFTSVGKLVHQVFGTAYGVLFSGVSWTMKIGIGILLTW | | |
| | LGLNSRSTSLSMTCIAVGMVTLYLGVMVQA | | |
| E | MRCIGISNRDFVEGVSGGSWVDIVLEHGSCVTTMAKNKPTLD | MG560144 | 2 |
| w/fusion | FELIKTEAKQPATLRKYCIEAKLTNTTTASRCPREGEPSLNEEQ | | |
| loop | DKRFVCKHSMVDRGRGNGCGRFGKGGIVTCAMFTCKKNME | | |
| mutations- | GKIVQPENLEYTIVITPHSGEENAVGNDTGKHGKEIKVTPQSSI | | |
| DENV2 | TEAELTGYGTVTMECSPRTGLDFNEMVLLQMENKAWLVHRQ | | |
| | WFLDLPLPWLPGADTQGSNWIQKETLVTFKNPHAKKQDVVV | | |
| | LGSQEGAMHTALTGATEIQMSSGNLLFTGHLKCRLRMDKLQL | | |
| | KGMSYSMCTGKFKVVKEIAETQHGTIVIRVQYEGDGSPCKIPF | | |
| | EIMDLEKRHVLGRLITVNPIVTEKDSPVNIEAEPPFGDSYIIIGV | | |
| | EPGQLKLSWFKKGSSIGQMFETTMRGAKRMAILGDTAWDFG | | |
| | SLGGVFTSIGKALHQVFGAIYGAAFSGVSWTMKILIGVVITWI | | |
| | GMNSRSTSLSVSLVLVGVVTLYLGVMVQA | | |
| E | MRCVGVGNRDFVEGLSGATWVDVVLEHGGCVTTMAKNKPT | MH822957 | 3 |
| w/fusion | LDIELQKTEATQLATLRKLCIEGKITNITTDSRCPREGEAVLPE | | |
| loop | EQDQNYVCKHTYVDRGRGNGCGRFGKGSLVTCAKFQCLEPI | | |
| mutations- | EGKVVQYENLKYTVIITVHTGDQHQVGNETQGVTAEITPQAS | | |
| DENV3 | TTEAILPEYGTLGLECSPRTGLDFNEMILLTMKNKAWMVHRQ | | |
| | WFFDLPLPWTSGATTETPTWNRKELLVTFKNAHAKKQEVVV | | |
| | LGSQEGAMHTALTGATEIQNSGGTSIFAGHLKCRLKMDKLEL | | |
| | KGMSYAMCTNTFVLKKEVSETQHGTILIKVEYKGEDAPCKIPF | | |
| | STEDGQGKAHNGRLITANPVVTKKEEPVNIEAEPPFGESNIVIG | | |
| | IGDNALKINWYKKGSSIGKMFEATARGARRMAILGDTAWDF | | |
| | GSVGGVLNSLGKMVHQIFGSAYTALFSGVSWVMKIGIGVLLT | | |
| | WIGLNSKNTSMSFSCIAIGIITLYLGAVVQA | | |
| E | MRCVGVGNRDFVEGVSGGAWVDLVLEHGGCVTTMAQGKPT | KU523872 | 4 |
| w/fusion | LDFELTKTTAKEVALLRTYCIEASISNITTATRCPREGEPYLKE | | |
| loop | EQDQQYICRRDVVDRGRGNGCGRFGKGGVVTCAKFSCSGKI | | |
| mutations- | TGNLVQIENLEYTVVVTVHNGDTHAVGNDTSNHGVTATITPR | | |
| DENV4 | SPSVEVKLPDYGELTLDCEPRSGIDFNEMILMKMKKKTWLVH | | |
| | KQWFLDLPLPWTAGADTSEVHWNYKERMVTFKVPHAKRQD | | |
| | VTVLGSQEGAMHSALAGATEVDSGDGNHMFAGHLKCKVRM | | |
| | EKLRIKGMSYTMCSGKFSIDKEMAETQHGTTVVKVKYEGAG | | |
| | APCKVPIEIRDVNKEKVVGRIISSTPFAENTNSVTNIELEPPFGD | | |
| | SYIVIGVGDSALTLHWFRKGSSIGKMFESTYRGAKRMAILGET | | |
| | AWDFGSVGGLFTSLGKAVHQVFGSVYTTMFGGVSWMIRILIG | | |
| | FLVLWIGTNSRNTSMAMTCIAVGGITLFLGFTVQA | | |
| E | IRCIGVSNRDFVEGMSGGTWVDVVLEHGGCVTVMAQDKPTV | KY241737 | 5 |
| w/fusion | DIELVTTTVSNMAEVRSYCYEASISDMASDSRCPREGEAYLD | | |
| loop | KQSDTQYVCKRTLVDRGRGNGCGRFGKGSLVTCAKFACSKK | | |
| mutations- | MTGKSIQPENLEYRIMLSVHGSQHSGMIVNDTGHETDENRAK | | |
| Zika | VEITPNSPRAEATLGGFGSLGLDCEPRTGLDFSDLYYLTMNNK | | |
| | HWLVHKEWFHDIPLPWHAGADTGTPHWNNKEALVEFKDAH | | |
| | AKRQTVVVLGSQEGAVHTALAGALEAEMDGAKGRLSSGHLK | | |
| | CRLKMDKLRLKGVSYSLCTAAFTFTKIPAETLHGTVTVEVQY | | |
| | AGTDGPCKVPAQMAVDMQTLTPVGRLITANPVITESTENSKM | | |
| | MLELDPPFGDSYIVIGVGEKKITHHWHRSGSTIGKAFEATVRG | | |
| | AKRMAVLGDTAWDFGSVGGALNSLGKGIHQIFGAAFKSLFG | | |
| | GMSWFSQILIGTLLMWLGLNTKNGSISLMCLALGGVLIFLSTA | | |
| | VSA | | |
| E | MRCVGIGNRDFVEGLSGATWVDVVLEHGSCVTTMAKNKPTL | MG721060 | 6 |
| DENV1 | DIELLKTEVTNPAVLRKLCIEAKISNTTTDSRCPTQGEATLVEE | | |
| (without | QDANFVCRRTFVDRGWGNGCGLFGKGSLLTCAKFKCVTKLE | | |
| fusion | GKIVQYENLKYSVIVTVHTGDQHQVGNETTEHGTIATITPQAP | | |
| loop | TSEIQLTDYGALTLDCSPRTGLDFNEMVLLTMKEKSWLVHKQ | | |
| mutations) | WFLDLPLPWTSGASTSQETWNRQDLLVTFKTAHAKKQEVVV | | |
| | LGSQEGAMHTALTGATEIQTSGTTTIFAGHLKCRLKMDKLTL | | |
| | KGVSYVMCTGSFKLEKEVAETQHGTVLVQVKYEGTDAPCKIP | | |
| | ISTQDEKGVTQNGRLITANPIVTDKEKPVNIETEPPFGESYIVIG | | |
| | AGEKALKLSWFKKGSSIGKMFEATARGARRMAILGDTAWDF | | |
| | GSIGGVFTSVGKLVHQVFGTAYGVLFSGVSWTMKIGIGILLTW | | |
| | LGLNSRSTSLSMTCIAVGMVTLYLGVMVQA | | |
| E | MRCIGISNRDFVEGVSGGSWVDIVLEHGSCVTTMAKNKPTLD | MG560144 | 7 |
| DENV2 | FELIKTEAKQPATLRKYCIEAKLTNTTTASRCPTQGEPSLNEEQ | | |
| (without | DKRFVCKHSMVDRGWGNGCGLFGKGGIVTCAMFTCKKNME | | |
| fusion | GKIVQPENLEYTIVITPHSGEENAVGNDTGKHGKEIKVTPQSSI | | |
| loop | TEAELTGYGTVTMECSPRTGLDFNEMVLLQMENKAWLVHRQ | | |
| mutations) | WFLDLPLPWLPGADTQGSNWIQKETLVTFKNPHAKKQDVVV | | |
| | LGSQEGAMHTALTGATEIQMSSGNLLFTGHLKCRLRMDKLQL | | |
| | KGMSYSMCTGKFKVVKEIAETQHGTIVIRVQYEGDGSPCKIPF | | |
| | EIMDLEKRHVLGRLITVNPIVTEKDSPVNIEAEPPFGDSYIIIGV | | |
| | EPGQLKLSWFKKGSSIGQMFETTMRGAKRMAILGDTAWDFG | | |
| | SLGGVFTSIGKALHQVFGAIYGAAFSGVSWTMKILIGVVITWI | | |
| | GMNSRSTSLSVSLVLVGVVTLYLGVMVQA | | |
| E | MRCVGVGNRDFVEGLSGATWVDVVLEHGGCVTTMAKNKPT | MH822957 | 8 |
| DENV3 | LDIELQKTEATQLATLRKLCIEGKITNITTDSRCPTQGEAVLPE | | |
| (without | EQDQNYVCKHTYVDRGWGNGCGLFGKGSLVTCAKFQCLEPI | | |
| fusion | EGKVVQYENLKYTVIITVHTGDQHQVGNETQGVTAEITPQAS | | |
| loop | TTEAILPEYGTLGLECSPRTGLDFNEMILLTMKNKAWMVHRQ | | |
| mutations) | WFFDLPLPWTSGATTETPTWNRKELLVTFKNAHAKKQEVVV | | |
| | LGSQEGAMHTALTGATEIQNSGGTSIFAGHLKCRLKMDKLEL | | |
| | KGMSYAMCTNTFVLKKEVSETQHGTILIKVEYKGEDAPCKIPF | | |
| | STEDGQGKAHNGRLITANPVVTKKEEPVNIEAEPPFGESNIVIG | | |
| | IGDNALKINWYKKGSSIGKMFEATARGARRMAILGDTAWDF | | |
| | GSVGGVLNSLGKMVHQIFGSAYTALFSGVSWVMKIGIGVLLT | | |
| | WIGLNSKNTSMSFSCIAIGIITLYLGAVVQA | | |
| E | MRCVGVGNRDFVEGVSGGAWVDLVLEHGGCVTTMAQGKPT | KU523872 | 9 |
| DENV4 | LDFELTKTTAKEVALLRTYCIEASISNITTATRCPTQGEPYLKE | | |
| (without | EQDQQYICRRDVVDRGWGNGCGLFGKGGVVTCAKFSCSGKI | | |
| fusion | TGNLVQIENLEYTVVVTVHNGDTHAVGNDTSNHGVTATITPR | | |
| loop | SPSVEVKLPDYGELTLDCEPRSGIDFNEMILMKMKKKTWLVH | | |
| mutations) | KQWFLDLPLPWTAGADTSEVHWNYKERMVTFKVPHAKRQD | | |
| | VTVLGSQEGAMHSALAGATEVDSGDGNHMFAGHLKCKVRM | | |
| | EKLRIKGMSYTMCSGKFSIDKEMAETQHGTTVVKVKYEGAG | | |
| | APCKVPIEIRDVNKEKVVGRIISSTPFAENTNSVTNIELEPPFGD | | |
| | SYIVIGVGDSALTLHWFRKGSSIGKMFESTYRGAKRMAILGET | | |
| | AWDFGSVGGLFTSLGKAVHQVFGSVYTTMFGGVSWMIRILIG | | |
| | FLVLWIGTNSRNTSMAMTCIAVGGITLFLGFTVQA | | |
| EZIKV | IRCIGVSNRDFVEGMSGGTWVDVVLEHGGCVTVMAQDKPTV | KY241737 | 10 |
| (without | DIELVTTTVSNMAEVRSYCYEASISDMASDSRCPTQGEAYLD | | |
| fusion | KQSDTQYVCKRTLVDRGWGNGCGLFGKGSLVTCAKFACSK | | |
| loop | KMTGKSIQPENLEYRIMLSVHGSQHSGMIVNDTGHETDENRA | | |
| mutations) | KVEITPNSPRAEATLGGFGSLGLDCEPRTGLDFSDLYYLTMNN | | |
| | KHWLVHKEWFHDIPLPWHAGADTGTPHWNNKEALVEFKDA | | |
| | HAKRQTVVVLGSQEGAVHTALAGALEAEMDGAKGRLSSGHL | | |
| | KCRLKMDKLRLKGVSYSLCTAAFTFTKIPAETLHGTVTVEVQ | | |
| | YAGTDGPCKVPAQMAVDMQTLTPVGRLITANPVITESTENSK | | |
| | MMLELDPPFGDSYIVIGVGEKKITHHWHRSGSTIGKAFEATVR | | |
| | GAKRMAVLGDTAWDFGSVGGALNSLGKGIHQIFGAAFKSLF | | |
| | GGMSWFSQILIGTLLMWLGLNTKNGSISLMCLALGGVLIFLST | | |
| | AVSA | | |
| NS3 for | SGVLWDVPSPPETQKAELEEGVYRIKQQGIFGKTQVGVGVQK | FJ182038 | 11 |
| PF | EGVFHTMWHVTRGAVLTYNGKRLEPNWASVKKDLISYGGG | | |
| | WRLSAQWQKGEEVQVIAVEPGKNPKNFQTMPGTFQTTTGEIG | | |
| | AIALDFKPGTSGSPIINREGKVVGLYGNGVVTKNGGYVSGIAQ | | |
| | TNAEPDGPTPELEEEMFKKRNLTIMDLHPGSGKTRKYLPAIVR | | |
| | EAIKRRLRTLILAPTRVVAAEMEEALKGLPIRYQTTATKSEHT | | |
| | GREIVDLMCHATFTMRLLSPVRVPNYNLIIMDEAHFTDPASIA | | |
| | ARGYISTRVGMGEAAAIFMTATPPGTADAFPQSNAPIQDEERD | | |
| | IPERSWNSGNEWITDFAGKTVWFVPSIKAGNDIANCLRKNGK | | |
| | KVIQLSRKTFDTEYQKTKLNDWDFVVTTDISEMGANFKADRV | | |
| | IDPRRCLKPVILTDGPERVILAGPMPVTAASAAQRRGRVGRNP | | |
| | QKENDQYIFMGQPLNNDEDHAHWTEAKMLLDNINTPEGIIPA | | |
| | LFEPEREKSAAIDGEYRLKGESRKTFVELMRRGDLPVWLAHK | | |
| | VASEGIKYTDRKWCFDGQRNNQILEENMDVEIWTKEG- | | |
| | EKKKLRPRWLDARTYSDPLALKEFKDFAAGRK | | |
| NS3 for | SGVLWDVPSPPETQKAELEEGVYRIKQQGIFGKTQVGVGVQK | LT898452 | 12 |
| PFZ | EGVFHTMWHVTRGAVLTHNGKRLEPNWASVKKDLISYGGG | | |
| | WRLSAQWQKGEEVQVIAVEPGKNPKNFQTMPGTFQTTTGEIG | | |
| | AIALDFKPGTSGSPIINKEGKVVGLYGNGVVTKNGGYVSGIAQ | | |
| | TNAEPDGPTPELEEEMFKKRNLTIMDLHPGSGKTRKYLPAIVR | | |
| | EAIKRRLRTLILAPTRVVAAEMEEALKGLPIRYQTTATKSEHT | | |
| | GREIVDLMCHATFTMRLLSPVRVPNYNLIIMDEAHFTDPASIA | | |
| | ARGYISTRVGMGEAAAIFMTATPPGTADAFPQSNAPIQDEERD | | |
| | IPERSWNSGNEWITDFAGKTVWFVPSIKAGNDIANCLRKNGK | | |
| | KVIQLSRKTFDTEYQKTKLNDWDFVVTTDISEMGANFKADRV | | |
| | IDPRRCLKPVILTDGPERVILAGPMPVTAASAAQRRGRVGRNP | | |
| | QKENDQYIFTGQPLNNDEDHAHWTEAKMLLDNINTPEGILPA | | |
| | LFEPEREKSAAIDGEYRLKGESRKTFVELMRRGDLPVWLAHK | | |
| | VASEGIKYTDRKWCFDGQRNNQILEENMDVEIWTKEG- | | |
| | EKKKLRPRWLDARTYSDPLALKEFKDFAAGRK | | |
| NS3 for | SGVLWDVPSPPETQKAELEEGVYRIKQQGIFGKTQVGVGVQK | KF824902 | 13 |
| PD | EGVFHTMWHVTRGAVLTHNGKRLEPNWASVKKDLISYGGG | | |
| | WRLSAQWQKGEEVQVIAVEPGRNPKNFQTMPGIFQTTTGEIG | | |
| | AIALDFKPGTSGSPIINREGKVVGLYGNGVVTKNGGYVSGIAQ | | |
| | ANAEPEGPTPELEEEMFKKRNLTIMDLHPGSGKTRKYLPAIVR | | |
| | EAIKRRLRTLILAPTRVVAAEMEEALKGLPIRYQTTATKSEHT | | |
| | GREIVVLMCHATFTMRLLSPVRVPNYNLIIMDEAHFTDPASIA | | |
| | ARGYISTRVGMGEAAAIFMTATPPGTAEAFPQSNAPIQDEERD | | |
| | IPERSWNSGNEWITDFVGKTVWFVPSIKAGNDIANCLRKNGK | | |
| | KVIQLSRKTFDTEYQKTKLNDWDFVVTTDISEMGANFKADRV | | |
| | IDPRRCLKPVILTDGPERVILAGPMPVTAASAAQRRGRVGRNP | | |
| | QKENDQYIFMGQPLNNDEDHAHWTEAKMLLDNINTPEGIIPA | | |
| | LFEPEREKSAAIDGEYRLKGESRKTFVELMRRGDLPVWLAHK | | |
| | VASEGIKYTDRKWCFDGERNNQILEENMDVEIWTKEGERKKL | | |
| | RPRWLDARTYSDPLALKEFKDFAAGRK | | |
| NS3 for | SGALWDVPAPKEVKKGETTDGVYRVMTRRLLGSTQVGVGV | KY241714 | 14 |
| PZ | MQEGVFHTMWHVTKGSALRSGEGRLDPYWGDVKQDLVSYC | | |
| | GPWKLDAAWDGHSEVQLLAVPPGERARNIQTLPGIFKTKDGD | | |
| | IGAVALDYPAGTSGSPILDKCGRVIGLYGNGVVIKNGSYVSAI | | |
| | TQGRREEETPVECFEPSMLKKKQLTVLDLHPGAGKTRRVLPEI | | |
| | VREAIKTRLRTVILAPTRVVAAEMEEALRGLPVRYMTTAVNV | | |
| | THSGTEIVDLMCHATFTSRLLQPTRVPNYNLYIMDEAHFTDPS | | |
| | SIAARGYISTRVEMGEAAAIFMTATPPGTRDAFPDSNSPIMDTE | | |
| | VEVPERAWSSGFDWVTDHSGKTVWFVPSVRNGNEIAACLTK | | |
| | AGKRVIQLSRKTFETEFQKTKHQEWDFVVTTDISEMGANFKA | | |
| | DRVIDSRRCLKPVILDG-- | | |
| | ERVILAGPMPVTHASAAQRRGRIGRNPNKPGDEYLYGGGCAE | | |
| | TDEDHAHWLEARMLLDNIYLQDGLIASLYRPEADKVAAIEGE | | |
| | FKLRTEQRKTFVELMKRGDLPVWLAYQVASAGITYTDRRWC | | |
| | FDGTTNNTIMEDSVPAEVWTRYG- | | |
| | EKRVLKPRWMDARVCSDHAALKSFKEFAAGKR | | |
| prM | FHLTTRGGEPHMIVSKQERGKSLLFKTSAGVNMCTLIAMDLG | MG721060 | 15 |
| DENV1 | ELCEDTMTYKCPRITEAEPDDVDCWCNATDTWVTYGTCSQT | | |
| | GEHRRDKRSVALAPHVGLGLETRTETWMSSEGAWRQIQKVE | | |
| | TWALRHPGFTVIALFLAHAIGTSITQKGIIFILLMLVTPSMA | | |
| prM | FHLTTRNGEPHMIVSRQEKGKSLLFKTEDGVNMCTLMAMDL | MG560144 | 16 |
| DENV2 | GELCEDTVTYNCPLLRQNEPEDIDCWCNSTSTWVTYGTCTAT | | |
| | GEHRREKRSVALVPHVGMGLETRTETWMSSEGAWKHAQRIE | | |
| | TWILRHPGFTIMAAILAYTIGTTYFQRVLIFILLTAVAPSMT | | |
| prM | FHLTSRDGEPRMIVGKNERGKSLLFKTASGINMCTLIAMDLGE | MH822957 | 17 |
| DENV3 | MCDDTVTYKCPHITEVEPEDIDCWCNLTSTWVTYGTCNQAGE | | |
| | HRRDKRSVALAPHVGMGLDTRTQTWMSAEGAWRQVEKVET | | |
| | WALRHPGFTILALFLAHYIGTSLTQKVVIFILLMLVTPSMT | | |
| prM | FHLSTRDGEPLMIVAKHERGRPLLFKTTEGINKCTLIAMDLGE | KU523872 | 18 |
| DENV4 | MCEDTVTYKCPLLVNTEPEDIDCWCNLTSTWVMYGTCTQSG | | |
| | ERRREKRSVALTPHSGMGLETRAETWMSSEGAWKHAQRVES | | |
| | WILRNPGFALLAGFMAHMIGQTGIQRTVFFVLMMLVAPSYG | | |
| prM Zika | AEVTRRGSAYYMYLDRSDAGEAISFPTTLGMNKCYIQIMDLG | KY241737 | 19 |
| | HMCDATMSYECPMLDEGVEPDDVDCWCNTTSTWVVYGTCH | | |
| | HKKGEARRSRRAVTLPSHSTRKLQTRSQTWLESREYTKHLIR | | |
| | VENWIFRNPGFALAAAAIAWLLGSSTSQKVIYLVMILLIAPAY | | |
| | S | | |
| M | SVALAPHVGLGLETRTETWMSSEGAWRQIQKVETWALRHPG | MG721060 | 20 |
| DENV1 | FTVIALFLAHAIGTSITQKGIIFILLMLVTPSMA | | |
| M | SVALVPHVGMGLETRTETWMSSEGAWKHAQRIETWILRHPG | MG560144 | 21 |
| DENV2 | FTIMAAILAYTIGTTYFQRVLIFILLTAVAPSMT | | |
| M | SVALAPHVGMGLDTRTQTWMSAEGAWRQVEKVETWALRHP | MH822957 | 22 |
| DENV3 | GFTILALFLAHYIGTSLTQKVVIFILLMLVTPSMT | | |
| M | SVALTPHSGMGLETRAETWMSSEGAWKHAQRVESWILRNPG | KU523872 | 23 |
| DENV4 | FALLAGFMAHMIGQTGIQRTVFFVLMMLVAPSYG | | |
| M Zika | AVTLPSHSTRKLQTRSQTWLESREYTKHLIRVENWIFRNPGFA | KY241737 | 24 |
| | LAAAAIAWLLGSSTSQKVIYLVMILLIAPAYS | | |

TABLE 3
mRNA Vaccine Sequences

| | | SEQ |
|---|---|---|
| | | ID |
| Name | Sequence | NO: |
| tPA prME | gatatcgcaccatggatgccatgaagagaggactgtgttgcgtgctgctgctgtgtggcgccgtgttcgtgagccccttc | 33 |
| WT | catttaaccacacgcaatggagaaccacacatgatcgtcagcagacaagagaaagggaaaagtctcttgtttaaaacag | |
| DENV2 | aggatggtgtgaacatgtgtaccctcatggccatggaccttggtgaactgtgtgaagacacagtcacttataactgtcctc | |
| | ttctcaggcagaatgaaccagaagacatagactgctggtgcaactccacgtccacatgggtaacttatgggacatgtact | |
| | gccacaggagaacacagaagggaaaaaagatcagtggcactcgtcccacatgtgggaatgggattggagacacgaa | |
| | ctgaaacatggatgtcatcagaaggggcctggaaacacgcccagagaattgaaacctggatcttgagacatccaggttt | |
| | taccataatggcagcaatcttggcatacaccataggaacaacatatttccaaagagtcctgattttcatcttactgacagct | |
| | gtcgctccttcaatgacaatgcgctgcataggaatatcaaacagagactttgtggaaggggtttcaggaggaagctggg | |
| | ttgacatagtcttagaacatggaagctgtgtgacaacaatggcaaaaaacaaaccaacattggactttgagctgataaaa | |
| | acagaagccaagcaacctgccactctaaggaagtactgtatagaggcaaagctgaccaacacaacaacagcatctcg | |
| | atgcccaacacaaggggaacccagcctaaacgaagagcaggacaaaaggttcgtctgcaaacattccatggtagaca | |
| | gaggatggggaaatggatgtggattatttggaaaaggaggcatcgtgacctgtgccatgttcacatgcaaaaaaaacat | |
| | ggaagggaaaatcgtgcaaccagaaaacctggaatacaccattgtgataacacctcactcaggggaagagaatgcag | |
| | ttggaaatgacacaggaaaacatggcaaggaaatcaaagtaacaccacagagttctattacagaagcagaactgacag | |
| | gctatggcaccgtcacgatggagtgctctccgagaacgggccttgacttcaatgagatggtgttgctgcaaatggaaaa | |
| | caaagcttggttggtgcacaggcaatggttcttagacctgccgttaccatggctgcccggagcggacacacaaggatc | |
| | aaattggatacagaaggagacattggtcaccttcaaaaatccccatgcaaagaaacaggatgttgttgttttaggatctca | |
| | agaaggggctatgcatacagcacttacaggggccacggaaatccagatgtcatcaggaaacctactgttcacaggaca | |
| | tctcaagtgcagactgagaatggacaaactacagctcaaaggaatgtcatactctatgtgtacagggaagtttaaagttgt | |
| | aaaggaaatagcagaaacacaacatggaacaatagttatcagagtacaatatgaaggggatggttctccatgtaaaatc | |
| | ccttttgagataatggacttggaaaaaagacatgtcttaggtcgcctgattacagtcaacccaattgtcacagaaaaggac | |
| | agcccagtcaacatagaagcagaacctccatttggagacagctacatcattataggagtagaaccgggacaactgaag | |
| | ctcagctggtttaagaaaggaagctcaatcggccaaatgttcgagacaacaatgagaggagcgaagagaatggctattt | |
| | taggtgacacagcctgggattttggatccctgggaggagtgttcacatctataggaaaggccctccaccaggtttttgga | |
| | gcaatctatggggctgccttcagcggggtctcatggactatgaaaatcctcataggagttgtcatcacatggataggaat | |
| | gaattcacgcagcacctcactgtccgtgtcgttagtattagtgggagtcgtgacattgtatttgggagtcatggtgcaggct | |
| | tgattaattgatcgatacagcagcaattggcaagctgcttacatagaaggcgcgcc | |
| tPA prME | MDAMKRGLCCVLLLCGAVFVSPFHLTTRNGEPHMIVSRQEKGKSLLFKTE | 34 |
| WT | DGVNMCTLMAMDLGELCEDTVTYNCPLLRQNEPEDIDCWCNSTSTWVTY | |
| DENV2 | GTCTATGEHRREKRSVALVPHVGMGLETRTETWMSSEGAWKHAQRIETWI | |
| (aa) | LRHPGFTIMAAILAYTIGTTYFQRVLIFILLTAVAPSMTMRCIGISNRDFVEG | |
| | VSGGSWVDIVLEHGSCVTTMAKNKPTLDFELIKTEAKQPATLRKYCIEAKL | |
| | TNTTTASRCPTQGEPSLNEEQDKRFVCKHSMVDRGWGNGCGLFGKGGIVT | |
| | CAMFTCKKNMEGKIVQPENLEYTIVITPHSGEENAVGNDTGKHGKEIKVTP | |
| | QSSITEAELTGYGTVTMECSPRTGLDFNEMVLLQMENKAWLVHRQWFLDL | |
| | PLPWLPGADTQGSNWIQKETLVTFKNPHAKKQDVVVLGSQEGAMHTALT | |
| | GATEIQMSSGNLLFTGHLKCRLRMDKLQLKGMSYSMCTGKFKVVKEIAET | |
| | QHGTIVIRVQYEGDGSPCKIPFEIMDLEKRHVLGRLITVNPIVTEKDSPVNIE | |
| | AEPPFGDSYIIIGVEPGQLKLSWFKKGSSIGQMFETTMRGAKRMAILGDTA | |
| | WDFGSLGGVFTSIGKALHQVFGAIYGAAFSGVSWTMKILIGVVITWIGMNS | |
| | RSTSLSVSLVLVGVVTLYLGVMVQA | |
| tPA prME | gatatcgcaccatggatgccatgaagagaggactgtgttgcgtgctgctgctgtgtggcgccgtgttcgtgagccccgc | 35 |
| WT ZIKV | ggaggtcactagacgtgggagtgcatactatatgtacttggacagaagcgatgctggggaggccatatcttttccaacc | |
| | acactggggatgaataagtgttatatacagatcatggatcttggacacatgtgtgatgccaccatgagctatgaatgccct | |
| | atgctggatgagggggtagaaccagatgacgtcgattgttggtgcaacacgacgtcaacttgggttgtgtacggaacct | |
| | gccatcacaaaaaaggtgaagcacggagatctagaagagctgtgacgctcccctcccattccactaggaagctgcaaa | |
| | cgcggtcgcagacctggttggagtcaagagaatacacaaagcacttgattagagtcgaaaattggatattcaggaaccc | |
| | tggcttcgcgttagcagcagctgccatcgcttggcttttgggaagctcaacgagccaaaaagtcatatacttggtcatgat | |
| | actgctgattgccccggcatacagcatcaggtgcataggagtcagcaatagggactttgtggaaggtatgtcaggtggg | |
| | acttgggttgatgttgtcttggaacatggaggttgtgtcaccgtaatggcacaggacaaaccgactgtcgacatagagct | |
| | ggttacaacaacagtcagcaacatggcggaggtaagatcctactgctatgaggcatcaatatcggacatggcttcggac | |
| | agccgctgcccaacacaaggtgaagcctaccttgacaagcaatcagatacccaatatgtctgcaaaagaacgttagtg | |
| | gacagaggctggggaaatggatgtggactttttggaaaagggagcctggtgacatgcgctaagtttgcatgctccaaga | |
| | aaatgaccgggaagagcatccagccagagaatctggagtaccggataatgctgtcagtccatggctcccagcacagt | |
| | gggatgatcgttaatgacacaggacatgaaactgatgagaatagagcgaaggttgagataacgcccaattcaccaaga | |
| | gccgaagccaccctggggggttttggaagcctaggacttgattgtgaaccgaggacaggccttgacttttcagatttgta | |
| | ttacttgactatgaataacaagcactggttggttcacaaggagtggttccacgacattccattaccttggcacgctggggc | |
| | agacaccggaactccacactggaacaacaaagaagcactggtagagttcaaggacgcacatgccaaaaggcaaact | |
| | gtcgtggttctagggagtcaagaaggagcagttcacacggcccttgctggagctctggaggctgagatggatggtgca | |
| | aagggaaggctgtcctctggccacttgaaatgtcgcctgaaaatggacaaacttagattgaagggcgtgtcatactcctt | |
| | gtgtaccgcagcgttcacattcaccaagatcccggctgaaacactgcacgggacagtcacagtggaggtacagtacgc | |
| | agggacagatggaccctgcaaggttccagctcagatggcggtggacatgcaaactctgaccccagttgggaggttgat | |
| | aaccgctaaccccgtaatcactgaaagcactgagaactctaagatgatgctggaacttgatccaccatttggggactctt | |
| | acattgtcataggagtcggggagaagaagatcacccaccactggcacaggagtggcagtaccattggaaaagcatttg | |
| | aagccactgtgagaggtgccaagagaatggcagtcttgggagacacagcctgggactttggatcagttggaggcgct | |
| | ctcaactcattgggcaagggcatccatcaaatttttggagcagctttcaaatcattgtttggaggaatgtcctggttctcaca | |
| | aattctcattggaacgttgctgatgtggttgggcctgaacacaaagaatggatctatttcccttatgtgcttggccttagggg | |
| | gagtgttgatcttcttatccacagccgtctctgcttgattaattgatcgatacagcagcaattggcaagctgcttacatagaa | |
| | ggcgcgcc | |
| tPA prME | MDAMKRGLCCVLLLCGAVFVSPAEVTRRGSAYYMYLDRSDAGEAISFPTT | 36 |
| WT ZIKV | LGMNKCYIQIMDLGHMCDATMSYECPMLDEGVEPDDVDCWCNTTSTWV | |
| (aa) | VYGTCHHKKGEARRSRRAVTLPSHSTRKLQTRSQTWLESREYTKHLIRVEN | |
| | WIFRNPGFALAAAAIAWLLGSSTSQKVIYLVMILLIAPAYSIRCIGVSNRDFV | |
| | EGMSGGTWVDVVLEHGGCVTVMAQDKPTVDIELVTTTVSNMAEVRSYCY | |
| | EASISDMASDSRCPTQGEAYLDKQSDTQYVCKRTLVDRGWGNGCGLFGK | |
| | GSLVTCAKFACSKKMTGKSIQPENLEYRIMLSVHGSQHSGMIVNDTGHETD | |
| | ENRAKVEITPNSPRAEATLGGFGSLGLDCEPRTGLDFSDLYYLTMNNKHWL | |
| | VHKEWFHDIPLPWHAGADTGTPHWNNKEALVEFKDAHAKRQTVVVLGSQ | |
| | EGAVHTALAGALEAEMDGAKGRLSSGHLKCRLKMDKLRLKGVSYSLCTA | |
| | AFTFTKIPAETLHGTVTVEVQYAGTDGPCKVPAQMAVDMQTLTPVGRLIT | |
| | ANPVITESTENSKMMLELDPPFGDSYIVIGVGEKKITHHWHRSGSTIGKAFE | |
| | ATVRGAKRMAVLGDTAWDFGSVGGALNSLGKGIHQIFGAAFKSLFGGMS | |
| | WFSQILIGTLLMWLGLNTKNGSISLMCLALGGVLIFLSTAVSA | |
| tPA prME | gatatcgcaccatggatgccatgaagagaggactgtgttgcgtgctgctgctgtgtggcgccgtgttcgtgagccccttt | 37 |
| fusion | cacctgactacccggaacggcgagccacacatgatcgtgagcagacaggagaagggcaagagcctcctgtttaaaa | |
| loop | ccgaggacggagtgaacatgtgcactctgatggccatggacctcggagagctgtgtgaggacaccgtgacctacaatt | |
| mutation | gccctctgctgcgccagaacgagcctgaggacatcgattgttggtgcaactctacttctacctgggtgacctacggaac | |
| DENV2 | atgtaccgccacaggcgagcataggcgagaaaaacgctctgtggccctggtgcctcacgtgggcatgggcctggag | |
| | accaggacagagacatggatgagctctgaaggcgcttggaagcacgcacagcggatcgagacatggatcctgcggc | |
| | atcctgggtttactatcatggccgctatccttgcctatactatcggcacaacatactttcagagggtgctgatctttatcct | |
| | gctgacagccgtggccccctctatgaccatgaggtgtattggcatcagcaacagagactttgtagagggcgtgtccgggg | |
| | gcagctgggtggacatcgtgctggagcacgggagctgcgtgactactatggccaaaaacaagcctacactggacttc | |
| | gagctgatcaagaccgaggccaagcaacccgccacactgaggaagtactgcatcgaggccaagctgaccaacacca | |
| | ccaccgctagcagatgtcctcgcgagggcgaaccttcactgaacgaggagcaggacaagagattcgtgtgcaagcac | |
| | agcatggtggacagagggcgggggaatggctgtggcaggttcgggaagggcgggatcgtgacatgcgccatgttta | |
| | catgcaagaagaacatggagggcaaaatcgtccagccagagaacctggaatataccatcgtgatcacccctcacagc | |
| | ggagaggagaacgctgtgggaaacgacacaggcaagcatgggaaggaaattaaggtgactccacagagcagcatc | |
| | acagaggctgagctgaccggctatggcaccgtgaccatggagtgcagcccaagaaccggcctcgatttcaatgagat | |
| | ggtcctgctgcagatggaaaacaaagcctggctggtgcacagacagtggttcctggacctccccctgccttggctgcc | |
| | aggagccgacacccaggggtccaactggatccagaaagagaccctggtgacattcaagaaccctcacgcaaaaaag | |
| | caggacgtggtggtgctcggaagccaggagggcgctatgcataccgctctgaccggcgccacagagattcagatga | |
| | gcagcggcaatctgctcttcaccggacacctgaagtgtcggctgaggatggacaagctgcagctgaagggaatgtcct | |
| | actccatgtgcaccggcaagttcaaggtggtgaaggaaattgctgagacccagcacggcaccatcgtgattagggtgc | |
| | agtatgagggtgacggaagcccatgcaagatcccattcgaaatcatggatctggagaagcggcacgtgctggggaga | |
| | ctgatcaccgtgaatcctatcgtgaccgagaaagactcccccgtgaacatcgaagccgagccacctttcggggattctt | |
| | atatcatcattggcgtggagcctggccagctgaagctgtcctggttcaagaagggaagctctatcggacagatgttcga | |
| | gacaaccatgagaggcgccaagcggatggccatcctgggtgacactgcctgggatttcgggagcctcggcggcgtgt | |
| | tcactagtattggcaaggctctgcatcaagtgttcggcgccatctacggcgctgcattcagcggcgtctcctggacaatg | |
| | aaaattctgatcggcgtggtgatcacatggataggcatgaacagcaggtctacctctctgagcgtctctctggtgctggtc | |
| | ggggtggtgacactgtatctgggcgtgatggtgcaggcctgattaattgatcgatacagcagcaattggcaagctgctta | |
| | catagaaggcgcgcc | |
| tPA prME | MDAMKRGLCCVLLLCGAVFVSPFHLTTRNGEPHMIVSRQEKGKSLLFKTE | 38 |
| fusion | DGVNMCTLMAMDLGELCEDTVTYNCPLLRQNEPEDIDCWCNSTSTWVTY | |
| loop | GTCTATGEHRREKRSVALVPHVGMGLETRTETWMSSEGAWKHAQRIETWI | |
| mutation | LRHPGFTIMAAILAYTIGTTYFQRVLIFILLTAVAPSMTMRCIGISNRDFVEG | |
| DENV2 | VSGGSWVDIVLEHGSCVTTMAKNKPTLDFELIKTEAKQPATLRKYCIEAKL | |
| (aa) | TNTTTASRCPREGEPSLNEEQDKRFVCKHSMVDRGRGNGCGRFGKGGIVT | |
| | CAMFTCKKNMEGKIVQPENLEYTIVITPHSGEENAVGNDTGKHGKEIKVTP | |
| | QSSITEAELTGYGTVTMECSPRTGLDFNEMVLLQMENKAWLVHRQWFLDL | |
| | PLPWLPGADTQGSNWIQKETLVTFKNPHAKKQDVVVLGSQEGAMHTALT | |
| | GATEIQMSSGNLLFTGHLKCRLRMDKLQLKGMSYSMCTGKFKVVKEIAET | |
| | QHGTIVIRVQYEGDGSPCKIPFEIMDLEKRHVLGRLITVNPIVTEKDSPVNIE | |
| | AEPPFGDSYIIIGVEPGQLKLSWFKKGSSIGQMFETTMRGAKRMAILGDTA | |
| | WDFGSLGGVFTSIGKALHQVFGAIYGAAFSGVSWTMKILIGVVITWIGMNS | |
| | RSTSLSVSLVLVGVVTLYLGVMVQA | |
| tPA prME | gatatcgcaccatggatgccatgaagagaggactgtgttgcgtgctgctgctgtgtggcgccgtgttcgtgagccccgc | 39 |
| fusion | tgaagtgaccagaagaggctccgcttactacatgtatctggaccggagcgatgccggcgaagctattagcttcccaacc | |
| loop | accctgggcatgaataagtgttacattcagatcatggacctgggccacatgtgcgacgccaccatgtcttacgagtgtcc | |
| mutation | tatgctggacgagggcgtggagcccgacgacgtggactgctggtgcaatactaccagcacctgggtggtgtacggca | |
| ZIKV | cctgccaccacaagaagggagaggccagacgctctaggagagcagtgaccctgccctcccactccacaaggaagct | |
| | gcagacccggtcccagacctggctcgagtctagggagtacactaagcacctgatcagagtcgagaactggatctttag | |
| | aaatcccgggttcgccctggccgccgccgccatcgcctggctcctggggtccagcacctctcagaaagtgatctatctg | |
| | gtgatgatcctgctgattgcccccgcttacagcatcagatgtatcggagtgagcaacagagacttcgtggagggcatga | |
| | gcggcgggacatgggtggacgtggtgctggagcacggcggctgtgtgaccgtgatggcccaggacaagcccactgt | |
| | tgacatcgagctggtcaccaccaccgtgagcaacatggccgaggtgcggtcatactgctacgaggccagcatcagcg | |
| | atatggcctctgacagtaggtgcccaagagagggcgaggcctatctggacaagcagtctgacacccagtacgtctgta | |
| | agcgcacccttgtggataggggtaggggcaatggctgcggcagatttggtaagggctccctggtaacttgtgcaaagtt | |
| | tgcttgcagtaagaagatgacaggcaagtctatccagcccgagaacctggaatacagaattatgctgtccgtgcacggc | |
| | agtcagcatagcggaatgatcgtgaacgacaccggacacgagacagatgagaacagggccaaggtcgagatcaca | |
| | cctaactcccccagggccgaggccaccctgggcgggttcggatctctgggcctggactgcgagcctagaacaggcct | |
| | ggacttcagcgacctgtattatctgacaatgaataacaagcactggctggtgcacaaggagtggttccacgacatccctc | |
| | tgccctggcacgccggagctgacaccgggacccctcactggaacaataaggaggcactggttgagttcaaggacgc | |
| | ccacgccaagcggcagaccgtggtggttctgggtagccaggagggcgccgtgcacaccgcactggctggcgccctc | |
| | gaggctgaaatggatggcgccaagggaaggctgagttccggtcacctgaagtgtaggctgaagatggacaagctga | |
| | ggctcaagggcgtgtcctacagcctgtgcacagccgccttcacattcaccaaaatccctgccgaaacactccacggga | |
| | ccgtgaccgtggaggtgcagtacgccgggaccgatggcccatgcaaggtgcctgcacagatggctgtggatatgcag | |
| | actctgacccctgtggggagactgattaccgccaatcccgtgatcactgaatcaactgagaacagcaagatgatgctgg | |
| | agctggatccacctttcggcgactcatacatcgtgattggcgtgggcgagaaaaagatcacccaccattggcaccgctc | |
| | tggcagtaccatcggcaaggccttcgaggcaacagtgagaggcgccaagaggatggccgtgctgggagataccgct | |
| | tgggacttcggctcagtgggaggcgctctgaacagcctcggaaagggcatccatcagatcttcggcgcagctttcaagt | |
| | ctctgttcgggggcatgtcatggttcagccagattctgatcggtacactgctgatgtggctggggctgaatactaagaac | |
| | gggagcatctccctgatgtgcctggctctgggaggggtgctgatcttcctcagcaccgccgtgagcgcctgattaattga | |
| | tcgatacagcagcaattggcaagctgcttacatagaaggcgcgcc | |
| tPA prME | MDAMKRGLCCVLLLCGAVFVSPAEVTRRGSAYYMYLDRSDAGEAISFPTT | 40 |
| fusion | LGMNKCYIQIMDLGHMCDATMSYECPMLDEGVEPDDVDCWCNTTSTWV | |
| loop | VYGTCHHKKGEARRSRRAVTLPSHSTRKLQTRSQTWLESREYTKHLIRVEN | |
| mutation | WIFRNPGFALAAAAIAWLLGSSTSQKVIYLVMILLIAPAYSIRCIGVSNRDFV | |
| ZIKV (aa) | EGMSGGTWVDVVLEHGGCVTVMAQDKPTVDIELVTTTVSNMAEVRSYCY | |
| | EASISDMASDSRCPREGEAYLDKQSDTQYVCKRTLVDRGRGNGCGRFGKG | |
| | SLVTCAKFACSKKMTGKSIQPENLEYRIMLSVHGSQHSGMIVNDTGHETDE | |
| | NRAKVEITPNSPRAEATLGGFGSLGLDCEPRTGLDFSDLYYLTMNNKHWL | |
| | VHKEWFHDIPLPWHAGADTGTPHWNNKEALVEFKDAHAKRQTVVVLGSQ | |
| | EGAVHTALAGALEAEMDGAKGRLSSGHLKCRLKMDKLRLKGVSYSLCTA | |
| | AFTFTKIPAETLHGTVTVEVQYAGTDGPCKVPAQMAVDMQTLTPVGRLIT | |
| | ANPVITESTENSKMMLELDPPFGDSYIVIGVGEKKITHHWHRSGSTIGKAFE | |
| | ATVRGAKRMAVLGDTAWDFGSVGGALNSLGKGIHQIFGAAFKSLFGGMS | |
| | WFSQILIGTLLMWLGLNTKNGSISLMCLALGGVLIFLSTAVSA | |
| tPA NS3 | gatatcgcaccatggatgccatgaagagaggactgtgttgcgtgctgctgctgtgtggcgccgtgttcgtgagcccctc | 41 |
| PanFlavi | cggcgttctatgggacgtacccagccccccagagacacagaaagcagaactggaagagggggtctataggatcaaa | |
| (D) | cagcaaggaatttttgggaaaacccaagtaggggttggagtacagaaagaaggagtcttccacaccatgtggcacgtt | |
| | acaagaggggcggtgttgacatataatgggaaaagactggaaccaaactgggctagtgtgaaaaaagatctgatttcat | |
| | acggaggaggatggagattgagcgcacaatggcaaaagggggaggaggtgcaggttattgccgtagagcctggga | |
| | agaacccaaagaactttcaaaccatgccaggcacttttcagactacaacaggggaaataggagcaattgcactggattt | |
| | caagcctggaacttcaggatctcctatcataaacagagagggaaaggtagtgggactgtatggcaatggagtggttaca | |
| | aagaatggtggctatgtcagcggaatagcgcaaacgaatgcagaaccagatggaccgacaccagaattggaagaag | |
| | agatgttcaaaaagcgaaatctaaccataatggatcttcatcctgggtcaggaaagacacggaaataccttccagctatt | |
| | gttagagaggcaatcaagagacgtctaagaactctaattttggcaccgacaagggtggttgcagctgagatggaagaa | |
| | gcattgaaagggctcccaataaggtaccaaacaacagcaacaaaatctgaacacacaggaagagagattgttgatcta | |
| | atgtgccacgcaacgttcacaatgcgcctgctgtcaccagttagggttccaaattataacttgataataatggatgaagcc | |
| | catttcacagacccagccagtatagcggctagagggtacatatcgactcgtgttggaatgggagaggcagccgcaattt | |
| | tcatgacagcaacgccccctggaacagctgatgcctttcctcagagcaacgctccaattcaagatgaagaaagggaca | |
| | taccggaacgctcatggaattcaggcaatgaatggataaccgacttcgctgggaaaacggtgtggtttgtccccagcatt | |
| | aaagccggaaatgacatagcaaactgcttgcgaaaaaacgggaaaaaggtcattcaacttagtaggaagacttttgaca | |
| | cagaatatcagaaaactaaactgaatgattgggacttcgtagtgacaactgacatttcagaaatgggggccaatttcaaa | |
| | gcagatagagtgatcgacccaagaagatgtctcaaaccagtgatcctgacagatggaccagagcgggtgatcctggct | |
| | ggaccaatgccagtcaccgcggcgagtgctgcgcaaaggagagggagagttggcaggaacccacaaaaagaaaat | |
| | gaccagtacatattcatgggccaacctctcaataatgatgaagaccacgctcactggacagaagcaaaaatgctgctgg | |
| | acaacattaatacaccagaagggatcataccagctctctttgagccagaaagggagaagtcagccgccatagacggtg | |
| | agtatcgcttgaaaggtgaatctaggaagactttcgtggaactcatgaggaggggtgaccttccagtctggttagcccat | |
| | aaagtagcatcagaagggatcaaatatacagatagaaaatggtgctttgatggacaacgtaataatcaaattttagagga | |
| | gaacatggatgtggaaatctggacaaaggaaggagaaaagaaaaaattgagacctaggtggcttgatgcccgcactta | |
| | ttcagatcccttagcactcaaggaattcaaggactttgcggctggcagaaagtgattaattgatcgatacagcagcaattg | |
| | gcaagctgcttacatagaaggcgcgcc | |
| tPA NS3 | MDAMKRGLCCVLLLCGAVFVSPSGVLWDVPSPPETQKAELEEGVYRIKQQ | 42 |
| PanFlavi | GIFGKTQVGVGVQKEGVFHTMWHVTRGAVLTYNGKRLEPNWASVKKDLI | |
| (D) (aa) | SYGGGWRLSAQWQKGEEVQVIAVEPGKNPKNFQTMPGTFQTTTGEIGAIA | |
| | LDFKPGTSGSPIINREGKVVGLYGNGVVTKNGGYVSGIAQTNAEPDGPTPE | |
| | LEEEMFKKRNLTIMDLHPGSGKTRKYLPAIVREAIKRRLRTLILAPTRVVAA | |
| | EMEEALKGLPIRYQTTATKSEHTGREIVDLMCHATFTMRLLSPVRVPNYNL | |
| | IIMDEAHFTDPASIAARGYISTRVGMGEAAAIFMTATPPGTADAFPQSNAPI | |
| | QDEERDIPERSWNSGNEWITDFAGKTVWFVPSIKAGNDIANCLRKNGKKVI | |
| | QLSRKTFDTEYQKTKLNDWDFVVTTDISEMGANFKADRVIDPRRCLKPVIL | |
| | TDGPERVILAGPMPVTAASAAQRRGRVGRNPQKENDQYIFMGQPLNNDED | |
| | HAHWTEAKMLLDNINTPEGIIPALFEPEREKSAAIDGEYRLKGESRKTFVEL | |
| | MRRGDLPVWLAHKVASEGIKYTDRKWCFDGQRNNQILEENMDVEIWTKE | |
| | GEKKKLRPRWLDARTYSDPLALKEFKDFAAGRK | |
| tPA NS3 | gatatcgcaccatggatgccatgaagagaggactgtgttgcgtgctgctgctgtgtggcgccgtgttcgtgagcccctc | 43 |
| PanFlavi | cggagtcctgtgggacgtgcctagcccacccgaaacccagaaggctgagctggaggagggcgtgtacagaattaag | |
| (Z) | cagcagggcatttttggcaagacccaggtgggggtgggcgtgcagaaggagggggtgttccacaccatgtggcacgt | |
| | gactcgaggagcagtgctcacacacaacggcaagaggctggagcccaactgggcttccgtgaagaaggacctgattt | |
| | cttatggcggagggtggagactgtccgcccagtggcagaagggagaggaggtgcaggtgatcgccgtggaacccg | |
| | gcaagaacccaaagaacttccagaccatgcctggcacattccagacaaccacaggcgagatcggagctatcgctctg | |
| | gacttcaagcctggcacctccggctctcccatcatcaacaaggagggcaaagtggtggggctgtacggaaacggcgt | |
| | cgtgaccaagaacggcggctatgtgagcggcatcgcccagacaaacgccgaacctgacggccctactcccgagcta | |
| | gaggaggaaatgttcaagaaacgcaacctgacaatcatggacctgcaccctgggtccggcaaaacaaggaagtacct | |
| | gcccgccatcgtgcgcgaggctatcaagaggagactgaggaccctgatcctggccccaacaagagtggtggccgcc | |
| | gagatggaagaggctctgaagggcctgcccatcagataccagaccaccgcaaccaagagcgagcacaccggcagg | |
| | gaaatcgtggacctcatgtgtcatgccacctttaccatgcgcctgctgtctccagtgagggtgcctaactacaacctgatc | |
| | atcatggacgaggcccatttcaccgaccctgcctccatcgctgcccggggctacatcagcacccgggtgggcatggg | |
| | cgaggctgccgccatcttcatgacagctaccccaccaggtacagccgatgccttccctcagagtaacgccccaatcca | |
| | ggacgaagaaagagacatccctgaaagatcatggaatagcggcaacgaatggatcacagactttgccggaaagacc | |
| | gtgtggttcgtgccttccatcaaggctggcaatgacatcgctaactgtctgaggaaaaatggaaagaaggtgatccagct | |
| | gagcaggaagacatttgacacagagtaccagaagacaaagctgaacgactgggacttcgtcgtgaccacagacatca | |
| | gcgaaatgggagccaacttcaaggccgacagggtgatcgaccccaggcggtgcctgaaacctgtcatcctgacagac | |
| | ggccccgagcgggtgatcctggcggggccaatgcccgtgactgccgccagcgcagctcagagacgcggcagagtg | |
| | ggccggaacccccagaaggaaaacgaccagtacatcttcacaggccagcccctgaacaatgacgaggatcacgccc | |
| | attggaccgaagccaaaatgctgctggacaacattaacacccctgagggcatcctgcccgccctgttcgagcctgaga | |
| | gagagaaatccgccgccatcgatggcgagtaccgactgaaaggggagtccagaaagacatttgtggaactgatgcgg | |
| | agaggcgacctgcccgtgtggctggcccacaaggtcgctagcgagggaatcaaatacacagaccgcaagtggtgctt | |
| | cgatggccagaggaataatcagatcctggaggagaatatggatgtggagatttggactaaggagggcgagaagaaga | |
| | agctgaggccacgatggctggacgccaggacatacagcgaccctctggctctgaaggagttcaaagacttcgccgca | |
| | ggcaggaagtgattaattgatcgatacagcagcaattggcaagctgcttacatagaaggcgcgcc | |
| tPA NS3 | MDAMKRGLCCVLLLCGAVFVSPSGVLWDVPSPPETQKAELEEGVYRIKQQ | 44 |
| PanFlavi | GIFGKTQVGVGVQKEGVFHTMWHVTRGAVLTHNGKRLEPNWASVKKDLI | |
| (Z) (aa) | SYGGGWRLSAQWQKGEEVQVIAVEPGKNPKNFQTMPGTFQTTTGEIGAIA | |
| | LDFKPGTSGSPIINKEGKVVGLYGNGVVTKNGGYVSGIAQTNAEPDGPTPE | |
| | LEEEMFKKRNLTIMDLHPGSGKTRKYLPAIVREAIKRRLRTLILAPTRVVAA | |
| | EMEEALKGLPIRYQTTATKSEHTGREIVDLMCHATFTMRLLSPVRVPNYNL | |
| | IIMDEAHFTDPASIAARGYISTRVGMGEAAAIFMTATPPGTADAFPQSNAPI | |
| | QDEERDIPERSWNSGNEWITDFAGKTVWFVPSIKAGNDIANCLRKNGKKVI | |
| | QLSRKTFDTEYQKTKLNDWDFVVTTDISEMGANFKADRVIDPRRCLKPVIL | |
| | TDGPERVILAGPMPVTAASAAQRRGRVGRNPQKENDQYIFTGQPLNNDED | |
| | HAHWTEAKMLLDNINTPEGILPALFEPEREKSAAIDGEYRLKGESRKTFVEL | |
| | MRRGDLPVWLAHKVASEGIKYTDRKWCFDGQRNNQILEENMDVEIWTKE | |
| | GEKKKLRPRWLDARTYSDPLALKEFKDFAAGRK | |
| tPA NS3 | gatatcgcaccatggatgccatgaagagaggactgtgttgcgtgctgctgctgtgtggcgccgtgttcgtgagcccctc | 45 |
| PanDENV | cggagtcctgtgggacgtgcctagcccacccgaaacccagaaggctgagctggaggagggcgtgtacagaattaag | |
| | cagcagggcatttttggcaagacccaggtgggggtgggcgtgcagaaggagggggtgttccacaccatgtggcacgt | |
| | gactcgaggagcagtgctcacacacaacggcaagaggctggagcccaactgggcttccgtgaagaaggacctgattt | |
| | cttatggcggagggtggagactgtccgcccagtggcagaagggagaggaggtgcaggtgatcgccgtggaacccg | |
| | gcaggaacccaaagaacttccagaccatgcctggcatattccagacaaccacaggcgagatcggagctatcgctctg | |
| | gacttcaagcctggcacctccggctctcccatcatcaacagggagggcaaagtggtggggctgtacggaaacggcgt | |
| | cgtgaccaagaacggcggctatgtgagcggcatcgcccaggcaaacgccgaacctgagggccctactcccgagcta | |
| | gaggaggaaatgttcaagaaacgcaacctgacaatcatggacctgcaccctgggtccggcaaaacaaggaagtacct | |
| | gcccgccatcgtgcgcgaggctatcaagaggagactgaggaccctgatcctggccccaacaagagtggtggccgcc | |
| | gagatggaagaggctctgaagggcctgcccatcagataccagaccaccgcaaccaagagcgagcacaccggcagg | |
| | gaaatcgtggtgctcatgtgtcatgccacctttaccatgcgcctgctgtctccagtgagggtgcctaactacaacctgatc | |
| | atcatggacgaggcccatttcaccgaccctgcctccatcgctgcccggggctacatcagcacccgggtgggcatggg | |
| | cgaggctgccgccatcttcatgacagctaccccaccaggtacagccgaggccttccctcagagtaacgccccaatcca | |
| | ggacgaagaaagagacatccctgaaagatcatggaatagcggcaacgaatggatcacagactttgtgggaaagaccg | |
| | tgtggttcgtgccttccatcaaggctggcaatgacatcgctaactgtctgaggaaaaatggaaagaaggtgatccagctg | |
| | agcaggaagacatttgacacagagtaccagaagacaaagctgaacgactgggacttcgtcgtgaccacagacatcag | |
| | cgaaatgggagccaacttcaaggccgacagggtgatcgaccccaggcggtgcctgaaacctgtcatcctgacagacg | |
| | gccccgagcgggtgatcctggcggggccaatgcccgtgactgccgccagcgcagctcagagacgcggcagagtgg | |
| | gccggaacccccagaaggaaaacgaccagtacatcttcatgggccagcccctgaacaatgacgaggatcacgccca | |
| | ttggaccgaagccaaaatgctgctggacaacattaacacccctgagggcatcatccccgccctgttcgagcctgagag | |
| | agagaaatccgccgccatcgatggcgagtaccgactgaaaggggagtccagaaagacatttgtggaactgatgcgga | |
| | gaggcgacctgcccgtgtggctggcccacaaggtcgctagcgagggaatcaaatacacagaccgcaagtggtgcttc | |
| | gatggcgaaaggaataatcagatcctggaggagaatatggatgtggagatttggactaaggagggcgagagaaagaa | |
| | gctgaggccacgatggctggacgccaggacatacagcgaccctctggctctgaaggagttcaaagacttcgccgcag | |
| | gcaggaagtgattaattgatcgatacagcagcaattggcaagctgcttacatagaaggcgcgcc | |
| tPA NS3 | MDAMKRGLCCVLLLCGAVFVSPSGVLWDVPSPPETQKAELEEGVYRIKQQ | 46 |
| PanDENV | GIFGKTQVGVGVQKEGVFHTMWHVTRGAVLTHNGKRLEPNWASVKKDLI | |
| (aa) | SYGGGWRLSAQWQKGEEVQVIAVEPGRNPKNFQTMPGIFQTTTGEIGAIAL | |
| | DFKPGTSGSPIINREGKVVGLYGNGVVTKNGGYVSGIAQANAEPEGPTPEL | |
| | EEEMFKKRNLTIMDLHPGSGKTRKYLPAIVREAIKRRLRTLILAPTRVVAAE | |
| | MEEALKGLPIRYQTTATKSEHTGREIVVLMCHATFTMRLLSPVRVPNYNLII | |
| | MDEAHFTDPASIAARGYISTRVGMGEAAAIFMTATPPGTAEAFPQSNAPIQ | |
| | DEERDIPERSWNSGNEWITDFVGKTVWFVPSIKAGNDIANCLRKNGKKVIQ | |
| | LSRKTFDTEYQKTKLNDWDFVVTTDISEMGANFKADRVIDPRRCLKPVILT | |
| | DGPERVILAGPMPVTAASAAQRRGRVGRNPQKENDQYIFMGQPLNNDEDH | |
| | AHWTEAKMLLDNINTPEGIIPALFEPEREKSAAIDGEYRLKGESRKTFVELM | |
| | RRGDLPVWLAHKVASEGIKYTDRKWCFDGERNNQILEENMDVEIWTKEGE | |
| | RKKLRPRWLDARTYSDPLALKEFKDFAAGRK | |
| tPA NS3 | gatatcgcaccatggatgccatgaagagaggactgtgttgcgtgctgctgctgtgtggcgccgtgttcgtgagccccag | 47 |
| PanVIKV | tggtgctctatgggatgtgcctgctcccaaggaagtaaaaaagggggagaccacagatggagtgtacagagtaatgac | |
| | tcgtagactgctaggttcaacacaagttggagtgggagtcatgcaagagggggtctttcacactatgtggcacgtcaca | |
| | aaaggatccgcgctgagaagcggtgaagggagacttgatccatactggggagatgtcaagcaggatctggtgtcatac | |
| | tgtggtccatggaagctagatgccgcctgggacgggcacagcgaggtgcagctcttggccgtgccccccggagaga | |
| | gagcgaggaacatccagactctgcccggaatatttaagacaaaggatggggacattggagcggttgcgctggactac | |
| | ccagcaggaacttcaggatctccaatcctagacaagtgtgggagagtgataggactctatggcaatggggtcgtgatca | |
| | aaaatgggagttatgttagtgccatcacccaagggaggagggaggaagagactcctgttgagtgcttcgagccttcgat | |
| | gctgaagaagaagcagctaactgtcttagacttgcatcctggagctgggaaaaccaggagagttcttcctgaaatagtc | |
| | cgtgaagccataaaaacaagactccgtactgtgatcttagctccaactagggttgtcgctgctgaaatggaggaagccct | |
| | tagagggcttccagtgcgttatatgacaacagcagtcaatgtcacccactctgggacagaaatcgttgacttaatgtgcc | |
| | atgccaccttcacttcacgtctactacagccaaccagagtccccaactataatctgtatattatggatgaggcccacttcac | |
| | agatccctcaagtatagcagcaagaggatacatttcaacaagggttgagatgggcgaggcggctgccatcttcatgacc | |
| | gccacgccaccaggaacccgtgacgcatttccggactccaactcaccaattatggacaccgaagtggaagtcccgga | |
| | gagagcctggagctcaggctttgattgggtgacggaccattctggaaaaacagtttggtttgttccaagcgtgaggaac | |
| | ggcaatgagatcgcagcttgtctgacgaaggctggaaaacgggtcatacagctcagcagaaagacttttgagacagag | |
| | ttccagaaaacaaaacatcaagagtgggactttgtcgtgacaactgacatttcagagatgggcgccaactttaaagctga | |
| | ccgtgtcatagattccaggagatgcctaaagccggtcatacttgatggcgagagagtcattctggctggacccatgcctg | |
| | tcacacatgccagcgctgcccagaggagggggcgcataggcaggaatcccaacaaacctggagatgagtatctgtat | |
| | ggaggtgggtgcgcagagactgatgaagaccatgcacactggcttgaagcaagaatgctccttgacaatatctacctcc | |
| | aagatggcctcatagcctcgctctatcgacctgaggccgacaaagtagcagccattgagggagagttcaagcttagga | |
| | cggagcaaaggaagacctttgtggaactcatgaaaagaggagatcttcctgtttggctggcctatcaggttgcatctgcc | |
| | ggaataacctacacagatagaagatggtgctttgatggcacgaccaacaacaccataatggaagacagtgtgccggca | |
| | gaggtgtggaccagatacggagagaaaagagtgctcaaaccgaggtggatggacgccagagtctgttcagatcatgc | |
| | ggccctgaagtcattcaaggagtttgccgctgggaaaagatgattaattgatcgatacagcagcaattggcaagctgctt | |
| | acatagaaggcgcgcc | |
| tPA NS3 | MDAMKRGLCCVLLLCGAVFVSPSGALWDVPAPKEVKKGETTDGVYRVM | 48 |
| PanZIKV | TRRLLGSTQVGVGVMQEGVFHTMWHVTKGSALRSGEGRLDPYWGDVKQ | |
| (aa) | DLVSYCGPWKLDAAWDGHSEVQLLAVPPGERARNIQTLPGIFKTKDGDIG | |
| | AVALDYPAGTSGSPILDKCGRVIGLYGNGVVIKNGSYVSAITQGRREEETPV | |
| | ECFEPSMLKKKQLTVLDLHPGAGKTRRVLPEIVREAIKTRLRTVILAPTRVV | |
| | AAEMEEALRGLPVRYMTTAVNVTHSGTEIVDLMCHATFTSRLLQPTRVPN | |
| | YNLYIMDEAHFTDPSSIAARGYISTRVEMGEAAAIFMTATPPGTRDAFPDSN | |
| | SPIMDTEVEVPERAWSSGFDWVTDHSGKTVWFVPSVRNGNEIAACLTKAG | |
| | KRVIQLSRKTFETEFQKTKHQEWDFVVTTDISEMGANFKADRVIDSRRCLK | |
| | PVILDGERVILAGPMPVTHASAAQRRGRIGRNPNKPGDEYLYGGGCAETDE | |
| | DHAHWLEARMLLDNIYLQDGLIASLYRPEADKVAAIEGEFKLRTEQRKTFV | |
| | ELMKRGDLPVWLAYQVASAGITYTDRRWCFDGTTNNTIMEDSVPAEVWT | |
| | RYGEKRVLKPRWMDARVCSDHAALKSFKEFAAGKR | |

### REFERENCES

1. Sridhar S, Luedtke A, Langevin E, Zhu M, Bonaparte M, Machabert T, Savarino S, Zambrano B, Moureau A, Khromava A, Moodie Z, Westling T, Mascarenas C, Frago C, Cortes M, Chansinghakul D, Noriega F, Bouckenooghe A, Chen J, Ng S P, Gilbert P B, Gurunathan S and DiazGranados C A. Effect of Dengue Serostatus on Dengue Vaccine Safety and Efficacy. N Engl J Med. 2018.
2. Bhatt S, Gething P W, Brady O J, Messina J P, Farlow A W, Moyes C L, Drake J M, Brownstein J S, Hoen A G, Sankoh O, Myers M F, George D B, Jaenisch T, Wint G R, Simmons C P, Scott T W, Farrar J J and Hay S I. The global distribution and burden of dengue. Nature. 2013; 496:504-7.
3. Sabchareon A, Wallace D, Sirivichayakul C, Limkittikul K, Chanthavanich P, Suvannadabba S, Jiwariyavej V, Dulyachai W, Pengsaa K, Wartel T A, Moureau A, Saville M, Bouckenooghe A, Viviani S, Tomieporth N G and Lang J. Protective efficacy of the recombinant, live-attenuated, CYD tetravalent dengue vaccine in Thai schoolchildren: a randomised, controlled phase 2b trial. Lancet (London, England). 2012; 380:1559-67.
4. Marchette N J, Halstead S B, Falkler W A, Jr., Stenhouse A and Nash D. Studies on the pathogenesis of dengue infection in monkeys. 3. Sequential distribution of virus in primary and heterologous infections. The Journal of infectious diseases. 1973; 128:23-30.
5. Halstead S B. In vivo enhancement of dengue virus infection in rhesus monkeys by passively transferred antibody. The Journal of infectious diseases. 1979; 140:527-33.
6. Zellweger R M, Prestwood T R and Shresta S. Enhanced infection of liver sinusoidal endothelial cells in a mouse model of antibody-induced severe dengue disease. Cell host & microbe. 2010; 7:128-39.
7. Balsitis S J, Williams K L, Lachica R, Flores D, Kyle J L, Mehlhop E, Johnson S, Diamond M S, Beatty P R and Harris E. Lethal antibody enhancement of dengue disease in mice is prevented by Fc modification. PLoS pathogens. 2010; 6:e1000790.
8. Katzelnick L C, Gresh L, Halloran M E, Mercado J C, Kuan G, Gordon A, Balmaseda A and Harris E. Antibody-dependent enhancement of severe dengue disease in humans. Science (New York, N.Y.). 2017; 358:929-932.
9. Salje H, Cummings D A T, Rodriguez-Barraquer I, Katzelnick L C, Lessler J, Klungthong C, Thaisomboonsuk B, Nisalak A, Weg A, Ellison D, Macareo L, Yoon I K, Jarman R, Thomas S, Rothman A L, Endy T and Cauchemez S. Reconstruction of antibody dynamics and infection histories to evaluate dengue risk. Nature. 2018; 557:719-723.
10. Mangada M M and Rothman A L. Altered cytokine responses of dengue-specific CD4+ T cells to heterologous serotypes. Journal of immunology (Baltimore, Md.: 1950). 2005; 175:2676-83.
11. Mongkolsapaya J, Dejnirattisai W, Xu X N, Vasanawathana S, Tangthawornchaikul N, Chairunsri A, Sawasdivorn S, Duangchinda T, Dong T, Rowland-Jones S, Yenchitsomanus P T, McMichael A, Malasit P and Screaton G. Original antigenic sin and apoptosis in the pathogenesis of dengue hemorrhagic fever. Nature medicine. 2003; 9:921-7.
12. Elong Ngono A and Shresta S. Immune Response to Dengue and Zika. Annual review of immunology. 2018.
13. Vannice K S, Durbin A and Hombach J. Status of vaccine research and development of vaccines for dengue. Vaccine. 2016; 34:2934-2938.
14. Amorim J H, dos Santos Alves R P, Bizerra R, Araujo Pereira S, Ramos Pereira L, Nascimento Fabris D L, Santos R A, Romano C M and de Souza Ferreira L C. Antibodies are not required to a protective immune response against dengue virus elicited in a mouse encephalitis model. Virology. 2016; 487:41-9.
15. Alves R, Pereira L R, Fabris D L N, Salvador F S, Santos R A, Zanotto P M A, Romano C M, Amorim J H and Ferreira L C S. Production of a Recombinant Dengue Virus 2 NS5 Protein and Potential Use as a Vaccine Antigen. Clin Vaccine Immunol. 2016; 23:460-469.
16. Maeda D, Batista M T, Pereira L R, de Jesus Cintra M, Amorim J H, Mathias-Santos C, Pereira S A, Boscardin S B, Silva S D R, Faquim-Mauro E L, Silveira V B, Oliveira D B L, Johnston S A, Ferreira L C S and Rodrigues J F. Adjuvant-Mediated Epitope Specificity and Enhanced Neutralizing Activity of Antibodies Targeting Dengue Virus Envelope Protein. Frontiers in immunology. 2017; 8:1175.
17. Zaneti A B, Yamamoto M M, Sulczewski F B, Almeida B D S, Souza H F S, Ferreira N S, Maeda D, Sales N S, Rosa D S, Ferreira L C S and Boscardin S B. Dendritic Cell Targeting Using a DNA Vaccine Induces Specific Antibodies and CD4(+) T Cells to the Dengue Virus Envelope Protein Domain III. Frontiers in immunology. 2019; 10:59.
18. Amorim J H, Diniz M O, Cariri F A, Rodrigues J F, Bizerra R S, Goncalves A J, de Barcelos Alves A M and de Souza Ferreira L C. Protective immunity to DENV2 after immunization with a recombinant NS1 protein using a genetically detoxified heat-labile toxin as an adjuvant. Vaccine. 2012; 30:837-45.
19. Azevedo A S, Goncalves A J, Archer M, Freire M S, Galler R and Alves A M. The synergistic effect of combined immunization with a DNA vaccine and chimeric yellow fever/dengue virus leads to strong protection against dengue. PLoS One. 2013; 8:e58357.
20. Azevedo A S, Yamamura A M, Freire M S, Trindade G F, Bonaldo M, Galler R and Alves A M. DNA vaccines against dengue virus type 2 based on truncate envelope protein or its domain III. PLoS One. 2011; 6:e20528.
21. Costa S M, Azevedo A S, Paes M V, Sarges F S, Freire M S and Alves A M. DNA vaccines against dengue virus based on the ns1 gene: the influence of different signal sequences on the protein expression and its correlation to the immune response elicited in mice. Virology. 2007; 358:413-23.
22. Costa S M, Paes M V, Barreto D F, Pinhao A T, Barth O M, Queiroz J L, Armoa G R, Freire M S and Alves A M. Protection against dengue type 2 virus induced in mice immunized with a DNA plasmid encoding the non-structural 1 (NS1) gene fused to the tissue plasminogen activator signal sequence. Vaccine. 2006; 24:195-205.
23. Costa S M, Yorio A P, Goncalves A J, Vidale M M, Costa E C, Mohana-Borges R, Motta M A, Freire M S and Alves A M. Induction of a protective response in mice by the dengue virus NS3 protein using DNA vaccines. PLoS One. 2011; 6:e25685.
24. Goncalves A J, Oliveira E R, Costa S M, Paes M V, Silva J F, Azevedo A S, Mantuano-Barradas M, Nogueira A C, Almeida C J and Alves A M. Cooperation between CD4+ T Cells and Humoral Immunity Is Critical for Protection against Dengue Using a DNA Vaccine Based on the NS1 Antigen. PLoS Negl Trop Dis. 2015; 9:e0004277.
25. Oliveira E R, Goncalves A J, Costa S M, Azevedo A S, Mantuano-Barradas M, Nogueira A C and Alves A M. Aspects of T Cell-Mediated Immunity Induced in Mice by a DNA Vaccine Based on the Dengue-NS1 Antigen after Challenge by the Intracerebral Route. PLoS One. 2016; 11:e0163240.
26. Fowler A M, Tang W W, Young M P, Mamidi A, Viramontes K M, McCauley M D, Carlin A F, Schooley R T, Swanstrom J, Baric R S, Govero J, Diamond M S and Shresta S. Maternally Acquired Zika Antibodies Enhance Dengue Disease Severity in Mice. Cell host & microbe. 2018; 24:743-750 e5.
27. Yauch L E, Zellweger R M, Kotturi M F, Qutubuddin A, Sidney J, Peters B, Prestwood T R, Sette A and Shresta S. A protective role for dengue virus-specific CD8+ T cells. Journal of immunology (Baltimore, Md.: 1950). 2009; 182:4865-73.
28. Yauch L E, Prestwood T R, May M M, Morar M M, Zellweger R M, Peters B, Sette A and Shresta S. CD4+ T cells are not required for the induction of dengue virus-specific CD8+ T cell or antibody responses but contribute to protection after vaccination. Journal of immunology (Baltimore, Md.: 1950). 2010; 185:5405-16.
29. Zellweger R M, Tang W W, Eddy W E, King K, Sanchez M C and Shresta S. CD8+ T Cells Can Mediate Short-Term Protection against Heterotypic Dengue Virus Reinfection in Mice. Journal of virology. 2015; 89:6494-505.
30. Wen J, Elong Ngono A, Angel Regla-Nava J, Kim K, Gorman M J, Diamond M S and Shresta S. Dengue virus-reactive CD8+ T cells mediate cross-protection against subsequent Zika virus challenge. Nat Commun. 2017; 8:1459.
31. Regla-Nava J A, Elong Ngono A, Viramontes K M, Huynh A T, Wang Y T, Nguyen A T, Salgado R, Mamidi A, Kim K, Diamond M S and Shresta S. Cross-reactive Dengue virus-specific CD8(+) T cells protect against Zika virus during pregnancy. Nat Commun. 2018; 9:3042.
32. Zellweger R M, Miller R, Eddy W E, White L J, Johnston R E and Shresta S. Role of humoral versus cellular responses induced by a protective dengue vaccine candidate. PLoS pathogens. 2013; 9:e1003723.
33. Zellweger R M, Eddy W E, Tang W W, Miller R and Shresta S. CD8+ T cells prevent antigen-induced antibody-dependent enhancement of dengue disease in mice. Journal of immunology (Baltimore, Md.: 1950). 2014; 193:4117-24.
34. Elong Ngono A, Chen H W, Tang W W, Joo Y, King K, Weiskopf D, Sidney J, Sette A and Shresta S. Protective Role of Cross-Reactive CD8 T Cells Against Dengue Virus Infection. EBioMedicine. 2016; 13:284-293.
35. Ashour J, Morrison J, Laurent-Rolle M, Belicha-Villanueva A, Plumlee C R, Bernal-Rubio D, Williams K L, Harris E, Fernandez-Sesma A, Schindler C and Garcia-Sastre A. Mouse STAT2 restricts early dengue virus replication. Cell host & microbe. 2010; 8:410-21.
36. Yu C Y, Chang T H, Liang J J, Chiang R L, Lee Y L, Liao C L and Lin Y L. Dengue virus targets the adaptor protein MITA to subvert host innate immunity. PLoS pathogens. 2012; 8:e1002780.
37. Aguirre S, Maestre A M, Pagni S, Patel J R, Savage T, Gutman D, Maringer K, Bernal-Rubio D, Shabman R S, Simon V, Rodriguez-Madoz J R, Mulder L C, Barber G N and Fernandez-Sesma A. DENV inhibits type I IFN production in infected cells by cleaving human STING. PLoS pathogens. 2012; 8:e1002934.
38. Grant A, Ponia S S, Tripathi S, Balasubramaniam V, Miorin L, Sourisseau M, Schwarz M C, Sanchez-Seco M P, Evans M J, Best S M and Garcia-Sastre A. Zika Virus Targets Human STAT2 to Inhibit Type I Interferon Signaling. Cell host & microbe. 2016; 19:882-90.
39. Ding Q, Gaska J M, Douam F, Wei L, Kim D, Balev M, Heller B and Ploss A. Species-specific disruption of STING-dependent antiviral cellular defenses by the Zika virus NS2B3 protease. Proceedings of the National Academy of Sciences of the United States of America. 2018; 115:E6310-E6318.
40. Pinto A K, Brien J D, Lam C Y, Johnson S, Chiang C, Hiscott J, Sarathy V V, Barrett A D, Shresta S and Diamond M S. Defining New Therapeutics Using a More Immunocompetent Mouse Model of Antibody-Enhanced Dengue Virus Infection. mBio. 2015; 6:e01316-15.
41. Shresta S, Sharar K L, Prigozhin D M, Beatty P R and Harris E. Murine model for dengue virus-induced lethal disease with increased vascular permeability. Journal of virology. 2006; 80:10208-17.
42. Tang W W, Grewal R and Shresta S. Influence of antibodies and T cells on dengue disease outcome: insights from interferon receptor-deficient mouse models. Current opinion in virology. 2015; 13:61-6.
43. Zellweger R M and Shresta S. Mouse models to study dengue virus immunology and pathogenesis. Frontiers in immunology. 2014; 5:151.
44. Prestwood T R, Morar M M, Zellweger R M, Miller R, May M M, Yauch L E, Lada S M and Shresta S. Gamma interferon (IFN-gamma) receptor restricts systemic dengue virus replication and prevents paralysis in IFN-alpha/beta receptor-deficient mice. Journal of virology. 2012; 86:12561-70.
45. Weiskopf D, Angelo M A, de Azeredo E L, Sidney J, Greenbaum J A, Fernando A N, Broadwater A, Kolla R V, De Silva A D, de Silva A M, Mattia K A, Doranz B J, Grey H M, Shresta S, Peters B and Sette A. Comprehensive analysis of dengue virus-specific responses supports an HLA-linked protective role for CD8+ T cells. Proceedings of the National Academy of Sciences of the United States of America. 2013; 110:E2046-53.
46. de Alwis R, Bangs D J, Angelo M A, Cerpas C, Fernando A, Sidney J, Peters B, Gresh L, Balmaseda A, de Silva A D, Harris E, Sette A and Weiskopf D. Immunodominant Dengue Virus-Specific CD8+ T Cell Responses Are Associated with a Memory PD-1+ Phenotype. Journal of virology. 2016; 90:4771-9.
47. Weiskopf D, Cerpas C, Angelo M A, Bangs D J, Sidney J, Paul S, Peters B, Sanches F P, Silvera C G, Costa P R, Kallas E G, Gresh L, de Silva A D, Balmaseda A, Harris E and Sette A. Human CD8+ T-Cell Responses Against the 4 Dengue Virus Serotypes Are Associated With Distinct Patterns of Protein Targets. The Journal of infectious diseases. 2015; 212:1743-51.
48. Simon-Loriere E, Duong V, Tawfik A, Ung S, Ly S, Casademont I, Prot M, Courtejoie N, Bleakley K, Buchy P, Tarantola A, Dussart P, Cantaert T and Sakuntabhai A. Increased adaptive immune responses and proper feedback regulation protect against clinical dengue. Sci Transl Med. 2017; 9.
49. Rivino L, Kumaran E A, Them T L, Too C T, Gan V C, Hanson B J, Wilder-Smith A, Bertoletti A, Gascoigne N R, Lye D C, Leo Y S, Akbar A N, Kemeny D M and MacAry P A. Virus-specific T lymphocytes home to the skin during natural dengue infection. Sci Transl Med. 2015; 7:278ra35.
50. Wijeratne D T, Fernando S, Gomes L, Jeewandara C, Ginneliya A, Samarasekara S, Wijewickrama A, Hardman C S, Ogg G S and Malavige G N. Quantification of dengue virus specific T cell responses and correlation with viral load and clinical disease severity in acute dengue infection. PLoS Negl Trop Dis. 2018; 12:e0006540.
51. Hatch S, Endy T P, Thomas S, Mathew A, Potts J, Pazoles P, Libraty D H, Gibbons R and Rothman A L. Intracellular cytokine production by dengue virus-specific T cells correlates with subclinical secondary infection. The Journal of infectious diseases. 2011; 203:1282-91.
52. Croft M. The TNF family in T cell differentiation and function—unanswered questions and future directions. Semin Immunol. 2014; 26:183-90.
53. Salek-Ardakani S and Croft M. Tumor necrosis factor receptor/tumor necrosis factor family members in antiviral CD8 T-cell immunity. J Interferon Cytokine Res. 2010; 30:205-18.
54. Salek-Ardakani S, Flynn R, Arens R, Yagita H, Smith G L, Borst J, Schoenberger S P and Croft M. The TNFR family members OX40 and CD27 link viral virulence to protective T cell vaccines in mice. J Clin Invest. 2011; 121:296-307.
55. Amorim J H, Pereira Bizerra R S, dos Santos Alves R P, Sbrogio-Almeida M E, Levi J E, Capurro M L and de Souza Ferreira L C. A genetic and pathologic study of a DENV2 clinical isolate capable of inducing encephalitis and hematological disturbances in immunocompetent mice. PLoS One. 2012; 7:e44984.
56. McDermott D S and Varga S M. Quantifying antigen-specific CD4 T cells during a viral infection: CD4 T cell responses are larger than we think. Journal of immunology (Baltimore, Md.: 1950). 2011; 187:5568-76.
57. Butler N S, Moebius J, Pewe L L, Traore B, Doumbo O K, Tygrett L T, Waldschmidt T J, Crompton P D and Harty J T. Therapeutic blockade of P D-L1 and LAG-3 rapidly clears established blood-stage Plasmodium infection. Nature immunology. 2011; 13:188-95.
58. Welten S P, Redeker A, Franken K L, Benedict C A, Yagita H, Wensveen F M, Borst J, Melief C J, van Lier R A, van Gisbergen K P and Arens R. CD27-CD70 costimulation controls T cell immunity during acute and persistent cytomegalovirus infection. Journal of virology. 2013; 87:6851-65.
59. Lam J H, Chua Y L, Lee P X, Martinez Gomez J M, Ooi E E and Alonso S. Dengue vaccine-induced CD8+ T cell immunity confers protection in the context of enhancing, interfering maternal antibodies. JCI Insight. 2017; 2.
60. Earl P L, Americo J L, Wyatt L S, Eller L A, Whitbeck J C, Cohen G H, Eisenberg R J, Hartmann C J, Jackson D L, Kulesh D A, Martinez M J, Miller D M, Mucker E M, Shamblin J D, Zwiers S H, Huggins J W, Jahrling P B and Moss B. Immunogenicity of a highly attenuated MVA smallpox vaccine and protection against monkeypox. Nature. 2004; 428:182-5.
61. Vezys V, Yates A, Casey K A, Lanier G, Ahmed R, Antia R and Masopust D. Memory CD8 T-cell compartment grows in size with immunological experience. Nature. 2009; 457:196-9.
62. Wirth T C, Xue H H, Rai D, Sabel J T, Bair T, Harty J T and Badovinac V P. Repetitive antigen stimulation induces stepwise transcriptome diversification but preserves a core signature of memory CD8(+) T cell differentiation. Immunity. 2010; 33:128-40.
63. Masopust D, Ha S J, Vezys V and Ahmed R. Stimulation history dictates memory CD8 T cell phenotype: implications for prime-boost vaccination. Journal of immunology (Baltimore, Md.: 1950). 2006; 177:831-9.
64. Jabbari A and Harty J T. Secondary memory CD8+ T cells are more protective but slower to acquire a central-memory phenotype. J Exp Med. 2006; 203:919-32.
65. Van Braeckel-Budimir N, Varga S M, Badovinac V P and Harty J T. Repeated Antigen Exposure Extends the Durability of Influenza-Specific Lung-Resident Memory CD8(+) T Cells and Heterosubtypic Immunity. Cell Rep. 2018; 24:3374-3382 e3.
66. Muthumani K, Griffin B D, Agarwal S, Kudchodkar S B, Reuschel E L, Choi H, Kraynyak K A, Duperret E K, Keaton A A, Chung C, Kim Y K, Booth S A, Racine T, Yan J, Morrow M P, Jiang J, Lee B, Ramos S, Broderick K E, Reed C C, Khan A S, Humeau L, Ugen K E, Park Y K, Maslow J N, Sardesai N Y, Joseph Kim J, Kobinger G P and Weiner D B. In vivo protection against ZIKV infection and pathogenesis through passive antibody transfer and active immunisation with a prMEnv DNA vaccine. NPJ Vaccines. 2016; 1:16021.
67. Dowd K A, Ko S Y, Morabito K M, Yang E S, Pelc R S, DeMaso C R, Castilho L R, Abbink P, Boyd M, Nityanandam R, Gordon D N, Gallagher J R, Chen X, Todd J P, Tsybovsky Y, Harris A, Huang Y S, Higgs S, Vanlandingham D L, Andersen H, Lewis M G, De La Barrera R, Eckels K H, Jarman R G, Nason M C, Barouch D H, Roederer M, Kong W P, Mascola J R, Pierson T C and Graham B S. Rapid development of a DNA vaccine for Zika virus. Science (New York, N.Y.). 2016; 354:237-240.
68. Wang P G, Kudelko M, Lo J, Siu L Y, Kwok K T, Sachse M, Nicholls J M, Bruzzone R, Altmeyer R M and Nal B. Efficient assembly and secretion of recombinant subviral particles of the four dengue serotypes using native prM and E proteins. PLoS One. 2009; 4:e8325.
69. Fibriansah G, Tan J L, Smith S A, de Alwis R, Ng T S, Kostyuchenko V A, Jadi R S, Kukkaro P, de Silva A M, Crowe J E and Lok S M. A highly potent human antibody neutralizes dengue virus serotype 3 by binding across three surface proteins. Nat Commun. 2015; 6:6341.
70. de Alwis R, Smith S A, Olivarez N P, Messer W B, Huynh J P, Wahala W M, White L J, Diamond M S, Baric R S, Crowe J E, Jr. and de Silva A M. Identification of human neutralizing antibodies that bind to complex epitopes on dengue virions. Proceedings of the National Academy of Sciences of the United States of America. 2012; 109:7439-44.
71. Rouvinski A, Guardado-Calvo P, Barba-Spaeth G, Duquerroy S, Vaney M C, Kikuti C M, Navarro Sanchez M E, Dejnirattisai W, Wongwiwat W, Haouz A, Girard-Blanc C, Petres S, Shepard W E, Despres P, Arenzana-Seisdedos F, Dussart P, Mongkolsapaya J, Screaton G R and Rey F A. Recognition determinants of broadly neutralizing human antibodies against dengue viruses. Nature. 2015; 520:109-13.
72. Sales N S, Silva J R, Aps L, Silva M O, Porchia B, Ferreira L C S and Diniz M O. In vivo electroporation enhances vaccine-mediated therapeutic control of human papilloma virus-associated tumors by the activation of multifunctional and effector memory CD8(+) T cells. Vaccine. 2017; 35:7240-7249.
73. Elong Ngono A, Young M P, Bunz M, Xu Z, Hattakam S, Vizcarra E, Regla-Nava J A, Tang W W, Yamabhai M, Wen J and Shresta S. CD4+ T cells promote humoral immunity and viral control during Zika virus infection. PLoS pathogens. 2019; 15:e1007474.
74. Elong Ngono A, Vizcarra E A, Tang W W, Sheets N, Joo Y, Kim K, Gorman M J, Diamond M S and Shresta S. Mapping and Role of the CD8+ T Cell Response During Primary Zika Virus Infection in Mice. Cell host & microbe. 2017; 21:35-46.
75. Wen J, Tang W W, Sheets N, Ellison J, Sette A, Kim K and Shresta S. Identification of Zika virus epitopes reveals immunodominant and protective roles for dengue virus cross-reactive CD8+ T cells. Nature microbiology. 2017; 2:17036.
76. Masopust D, Ha S J, Vezys V and Ahmed R. Stimulation history dictates memory CD8 T cell phenotype: implications for prime-boost vaccination. Journal of immunology. 2006; 177:831-9.
77. Miller J D, van der Most R G, Akondy R S, Glidewell J T, Albott S, Masopust D, Murali-Krishna K, Mahar P L, Edupuganti S, Lalor S, Germon S, Del Rio C, Mulligan M J, Staprans S I, Altman J D, Feinberg M B and Ahmed R. Human effector and memory CD8+ T cell responses to smallpox and yellow fever vaccines. Immunity. 2008; 28:710-22.
78. Kaech S M and Wherry E J. Heterogeneity and cell-fate decisions in effector and memory CD8+ T cell differentiation during viral infection. Immunity. 2007; 27:393-405.
79. Gerlach C, Moseman E A, Loughhead S M, Alvarez D, Zwijnenburg A J, Waanders L, Garg R, de la Torre J C and von Andrian U H. The Chemokine Receptor CX3CR1 Defines Three Antigen-Experienced CD8 T Cell Subsets with Distinct Roles in Immune Surveillance and Homeostasis. Immunity. 2016; 45:1270-1284.
80. Jiang X, Clark R A, Liu L, Wagers A J, Fuhlbrigge R C and Kupper T S. Skin infection generates non-migratory memory CD8+ T(RM) cells providing global skin immunity. Nature. 2012; 483:227-31.
81. Steinert E M, Schenkel J M, Fraser K A, Beura L K, Manlove L S, Igyarto B Z, Southern P J and Masopust D. Quantifying Memory CD8 T Cells Reveals Regionalization of Immunosurveillance. Cell. 2015; 161:737-49.
82. Mueller S N, Gebhardt T, Carbone F R and Heath W R. Memory T cell subsets, migration patterns, and tissue residence. Annu Rev Immunol. 2013; 31:137-61.
83. Milner J J, Toma C, Yu B, Zhang K, Omilusik K, Phan A T, Wang D, Getzler A J, Nguyen T, Crotty S, Wang W, Pipkin M E and Goldrath A W. Runx3 programs CD8(+) T cell residency in non-lymphoid tissues and tumours. Nature. 2017; 552:253-257.
84. Anderson K G, Mayer-Barber K, Sung H, Beura L, James B R, Taylor J J, Qunaj L, Griffith T S, Vezys V, Barber D L and Masopust D. Intravascular staining for discrimination of vascular and tissue leukocytes. Nat Protoc. 2014; 9:209-22.
85. Grifoni A, Pham J, Sidney J, O'Rourke P H, Paul S, Peters B, Martini S R, de Silva A D, Ricciardi M J, Magnani D M, Silveira C G T, Maestri A, Costa P R, de-Oliveira-Pinto L M, de Azeredo E L, Damasco P V, Phillips E, Mallal S, de Silva A M, Collins M, Durbin A, Diehl S A, Cerpas C, Balmaseda A, Kuan G, Coloma J, Harris E, Crowe J E, Jr., Stone M, Norris P J, Busch M, Vivanco-Cid H, Cox J, Graham B S, Ledgerwood J E, Turtle L, Solomon T, Kallas E G, Watkins D I, Weiskopf D and Sette A. Prior Dengue virus exposure shapes T cell immunity to Zika virus in humans. Journal of virology. 2017.
86. Weiskopf D, Angelo M A, Sidney J, Peters B, Shresta S and Sette A. Immunodominance changes as a function of the infecting dengue virus serotype and primary versus secondary infection. Journal of virology. 2014; 88:11383-94.
87. Weiskopf D, Angelo M A, Bangs D J, Sidney J, Paul S, Peters B, de Silva A D, Lindow J C, Diehl S A, Whitehead S, Durbin A, Kirkpatrick B and Sette A. The human CD8+ T cell responses induced by a live attenuated tetravalent dengue vaccine are directed against highly conserved epitopes. Journal of virology. 2015; 89:120-8.
88. Nolz J C and Harty J T. Protective capacity of memory CD8+ T cells is dictated by antigen exposure history and nature of the infection. Immunity. 2011; 34:781-93.
89. Davis B S, Chang G J, Cropp B, Roehrig J T, Martin D A, Mitchell C J, Bowen R and Bunning M L. West Nile virus recombinant DNA vaccine protects mouse and horse from virus challenge and expresses in vitro a noninfectious recombinant antigen that can be used in enzyme-linked immunosorbent assays. Journal of virology. 2001; 75:4040-7.
90. Chang G J, Hunt A R, Holmes D A, Springfield T, Chiueh T S, Roehrig J T and Gubler D J. Enhancing biosynthesis and secretion of premembrane and envelope proteins by the chimeric plasmid of dengue virus type 2 and Japanese encephalitis virus. Virology. 2003; 306:170-80.
91. van Gisbergen K P, Klarenbeek P L, Kragten N A, Unger P P, Nieuwenhuis M B, Wensveen F M, ten Brinke A, Tak P P, Eldering E, Nolte M A and van Lier R A. The costimulatory molecule CD27 maintains clonally diverse CD8(+) T cell responses of low antigen affinity to protect against viral variants. Immunity. 2011; 35:97-108.
92. Rowley T F and Al-Shamkhani A. Stimulation by soluble CD70 promotes strong primary and secondary CD8+ cytotoxic T cell responses in vivo. Journal of immunology (Baltimore, Md: 1950). 2004; 172:6039-46.
93. Weiskopf D, Yauch L E, Angelo M A, John D V, Greenbaum J A, Sidney J, Kolla R V, De Silva A D, de Silva A M, Grey H, Peters B, Shresta S and Sette A. Insights into HLA-restricted T cell responses in a novel mouse model of dengue virus infection point toward new implications for vaccine design. Journal of immunology. 2011; 187:4268-79.
94. Thompson J D, Higgins D G and Gibson T J. CLUSTAL W: improving the sensitivity of progressive multiple sequence alignment through sequence weighting, position-specific gap penalties and weight matrix choice. Nucleic Acids Res. 1994; 22:4673-80.
95. Milligan G N, Sarathy V V, Infante E, Li L, Campbell G A, Beatty P R, Harris E, Barrett A D and Bourne N. A Dengue Virus Type 4 Model of Disseminated Lethal Infection in AG129 Mice. PLoS One. 2015; 10:e0125476.
96. White L J, Sariol C A, Mattocks M D, Wahala M P B W, Yingsiwaphat V, Collier M L, Whitley J, Mikkelsen R, Rodriguez I V, Martinez M I, de Silva A and Johnston R E. An alphavirus vector-based tetravalent dengue vaccine induces a rapid and protective immune response in macaques that differs qualitatively from immunity induced by live virus infection. Journal of virology. 2013; 87:3409-24.
97. Milner J J and Goldrath A W. Transcriptional programming of tissue-resident memory CD8(+) T cells. Curr Opin Immunol. 2018; 51:162-169.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Development of a Vaccine for Cross-Protection Against Various Flavivirus Serotypes or Species

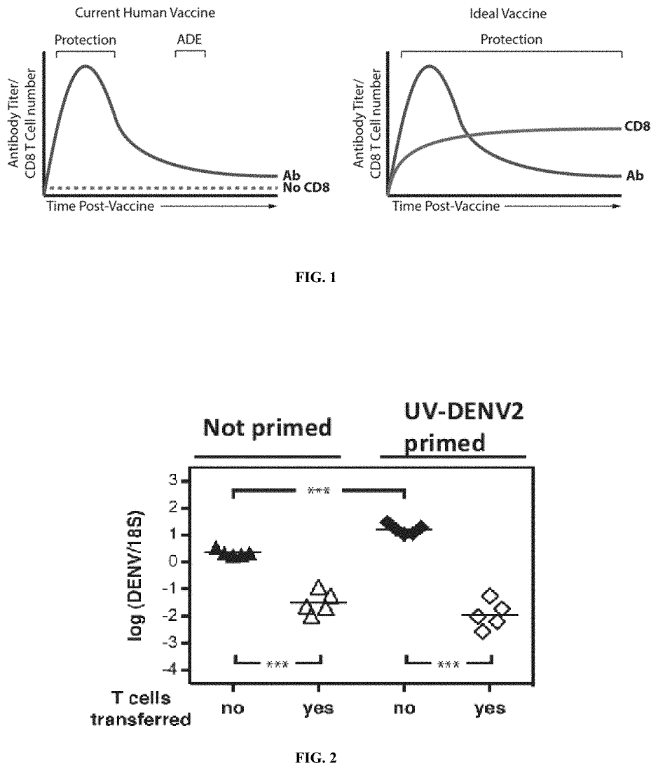

Example 1 refers to the results shown in FIG. 1 to FIG. 14.

Example 1 can be summarized as follows:

The Applicant describes experimental results and line of reasoning which supports the development of a vaccine for cross-protection against various flavivirus serotypes or flavivirus species.

Infection with DENV causes a spectrum of disease ranging from an acute, self-limited febrile illness (DF) to the life-threatening severe dengue, also known as dengue hemorrhagic fever/dengue shock syndrome (DHF/DSS). Signs and symptoms of DF consist of fever, retro-orbital headache, rash, and arthralgia. Severe dengue is characterized by increased vascular permeability, hemorrhagic manifestations, thrombocytopenia, and shock. The four serotypes of DENV (DENV1-4) are transmitted to humans primarily by the mosquitoes Aedes aegypti and Aedes albopictus. Each year, DENV causes an estimated 390 million infections, of which 500,000 cases develop into severe dengue². At present, a DENV-specific antiviral therapy or vaccine that is safe, effective, and widely available is lacking. Dengvaxia®, the only DENV vaccine approved for use in some countries, has multiple drawbacks, including lack of efficacy in DENV-naïve individuals and higher incidence of severe dengue disease in some vaccinated relative to unvaccinated individuals^{1, 3}.

Two central questions in the DENV field are: Why do secondary infections with DENV result in more severe illness than primary infections? Why do infants born to DENV-immune but not DENV-naïve mothers develop severe disease? To explain these epidemiological observations, two dominant hypotheses have been postulated: (i) antibody (Ab) dependent enhancement of infection (ADE) and (ii) original T cell antigenic sin. According to the ADE hypothesis, postulated almost 50 years ago, DENV-Ab complexes are formed and bind to Fcγ receptors on cells, facilitating viral entry and replication; increased viral burden resulting from ADE then drives the production of inflammatory mediators that increase vascular permeability^{4, 5}. The relevance of ADE to DHF/DSS pathogenesis had been controversial until the demonstration of ADE in vivo in 2010^{6, 7}. Recent evidence from cohort studies in DENV-endemic countries^{8, 9} and from clinical trials of Dengvaxia®¹ further support a major role for ADE in DENV pathogenesis.

According to the original T cell antigenic sin hypothesis, postulated over 30 years ago, serotype-cross-reactive memory T cells exhibit an altered phenotype in terms of cytokine production and degranulation, thereby contributing to the pathogenesis of severe denguel^{10, 11}. However, direct evidence for the pathogenic role of T cells during DENV infection is as yet lacking, and results obtained from studies using mouse models support an important contribution of T cells (both serotype-specific and -cross-reactive) to protection against DENV¹² (discussed below). In particular, Applicants have revealed that the interplay between the Ab and T cell responses influences the outcome of DENV infection and that CD8 T cells can actually prevent ADE. Consistent with mouse studies, Applicant's recent studies with human samples implicate a protective role for T cells against DENV and Zika Virus.

A long-term solution against the global DENV and ZIKV problem is the development of a safe and effective vaccine. Applicants have found a protective role for T cells, in particular CD8 T cells, against DENV and ZIKV infection, and set forth in the present application that CD8 T cell responses dictate the extent of dengue vaccine-mediated protection. To overcome the shortcomings of other available flavivirus vaccines (FIG. 1), Applicants herein describe a vaccine that elicits durable high-titer nAb and protective CD8 T cell responses against DENV serotypes, as well as against ZIKV. Further embodiments of the present invention provide protective immune responses against at least two flavivirus serotypes and/or flavivirus species.

Using mice lacking type I and/or type II IFN receptors, Applicants previously showed that the Ab response induced by a subunit DENV vaccine candidate or UV-irradiated DENV could mediate ADE whereas CD8 T cells protected even in the presence of ADE (FIG. 2)^{32, 33}. These results show that an interplay between Ab and CD8 T cell responses determines the outcome of DENV infection.

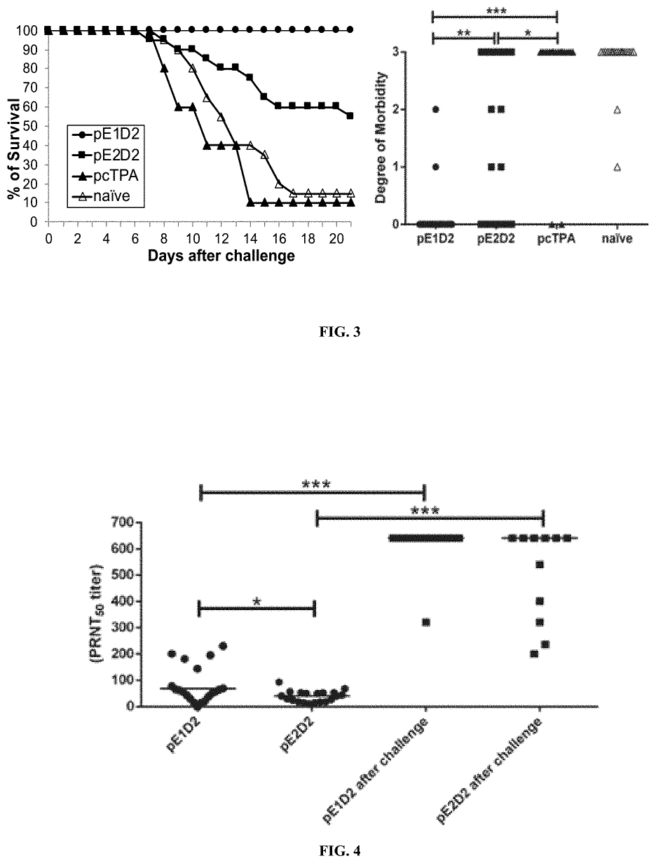

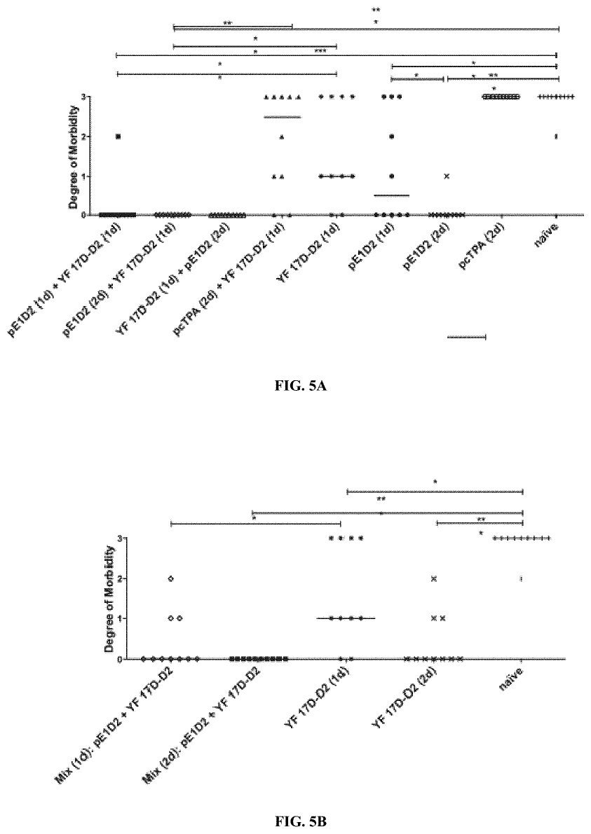

Applicants examined two DNA vaccines encoding the ectodomain (domains I, II and III) of the DENV2 E protein (pE1D2) or only domain III (pE2D2)²⁰. Briefly, BALB/C mice were immunized with these vaccines and then challenged via the intracerebral (i.c.) route with approximately 4 LD₅₀ (4.3 log₁₀ PFU) of the mouse brain-adapted DENV2 strain New Guinea C (NGC). All mice immunized with the pE1D2 vaccine survived the viral challenge (FIG. 3A), with low morbidity rates (10%) and mild degree (FIG. 3B). Immunization with the DNA vaccine encoding only E domain III was not as efficient, since 45% of animals died after virus challenge (FIG. 3A). Consistent with these efficacy data, levels of pre-challenge nAbs were significantly higher in pE1D2-vaccinated mice than in pE2D2-immunized animals (FIG. 4). Viral challenge induced a significant increase in the levels of the anti-DENV2 nAbs in the pE1D2-vaccinated group, as most mice in this group (95%) reached the maximal neutralization titer used in our assay (1≥640). In contrast, pE2D2-vaccinated animals that survived DENV2 challenge (11 mice) presented a broad range of nAb levels after challenge, and only six out of these 11 mice (55%) showed maximum PRNT titer. These results show that immunization with the DNA vaccine encoding the entire ectodomain of the DENV2 E protein afforded better protection against DENV2 than the vaccine encoding DENV2 E domain III.

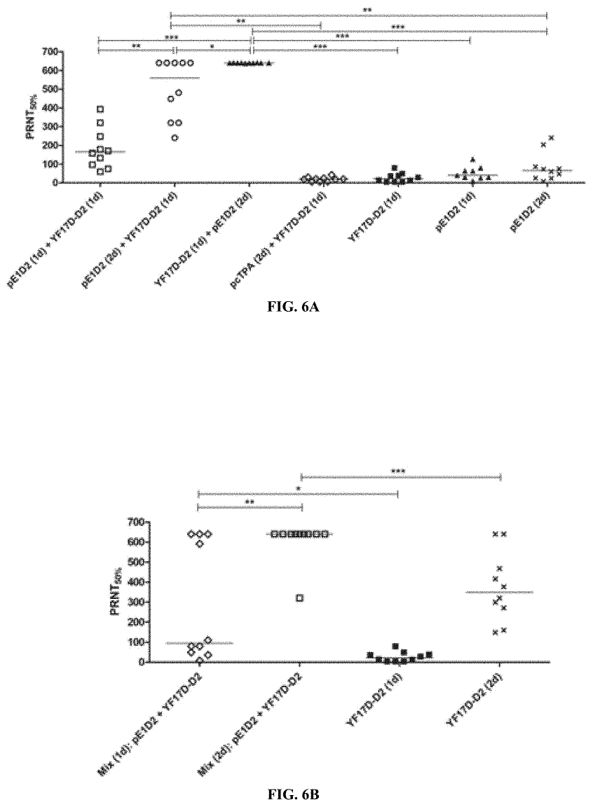

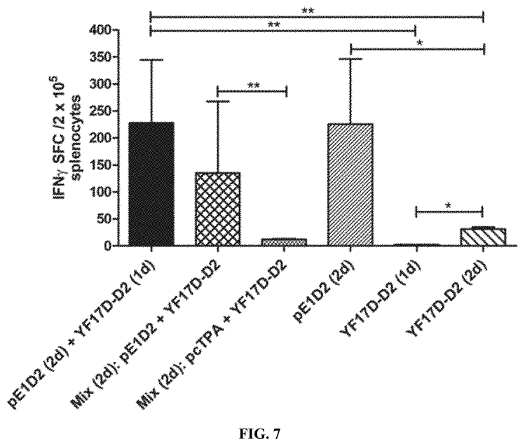

Applicants next combined immunization with the pE1D2 DNA plasmid with a chimeric YFV/DENV live-attenuated virus, named YF17D-D2, in which the prM and E proteins of YFV were substituted by DENV2 proteins¹⁹. The YF17D-D2 is similar to the DENV2 virus of Dengvaxia®. Mice immunized with these two vaccines using either a prime/boost or simultaneous injection protocol were fully protected without presenting any clinical sign of the DENV infection (FIGS. 5A-5B). It was observed that the two vaccines, in combination, induced higher levels of nAb responses against DENV2 as compared with the DNA or chimeric vaccine alone (FIGS. 6A-6B). In contrast, the DNA vaccine but not the chimeric virus vaccine elicited robust virus-specific CD8 T cell responses (FIG. 7). These results highlighted the efficient nature of DENV DNA vaccines in inducing the anti-DENV T cell response.

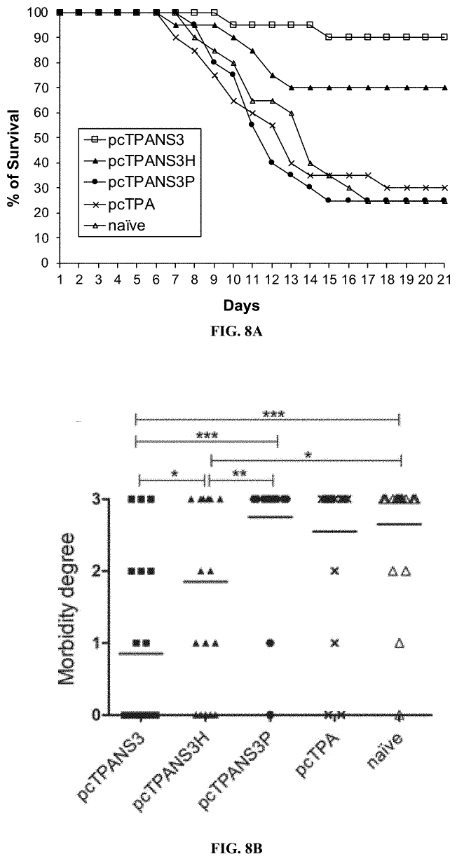

Applicants also evaluated the protective efficacy of DNA vaccines encoding DENV2 NS3²³. DNA plasmids encoding either the full-length NS3 (pcTPANS3) or only the protease and helicase domain (pcTPANS3H and pcTPANS3P, respectively) were constructed. BALB/c mice were immunized (i.m. route) with these DNA vaccines (100 μg of DNA, given two weeks apart) and then challenged via the i.c. route with DENV2 NGC DENV2 (40 LD₅₀) (FIGS. 8A-AB). Mice vaccinated with the plasmid encoding the protease domain (pcTPANS3P) exhibited similar mortality and morbidity as control mice (naïve or control plasmid-immunized mice), indicating that vaccination with the NS3 protease domain did not provide any protection. In contrast, animals immunized with the plasmid encoding the full-length NS3 or the helicase domain survived the viral challenge, albeit protection was partial (several of these vaccinated mice manifested hind leg paralysis and hunched posture). These data demonstrate that a NS3-based vaccine can provide protection against DENV. Altogether, these results with DNA-based DENV2 vaccines have established the foundation for the generation and testing of combination DENV1-4 and ZIKV prME and consensus NS3 DNA vaccines described herein.

### CD27 is Important for Generation of DENV-Specific CD8 T Cell Responses and Protection Against DENV-Induced Disease in Mice.

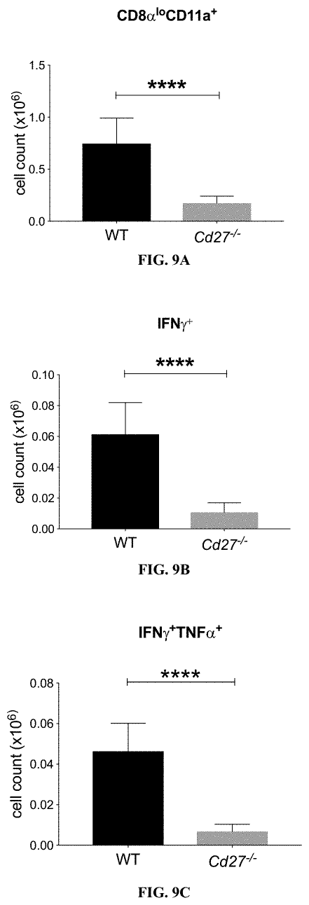

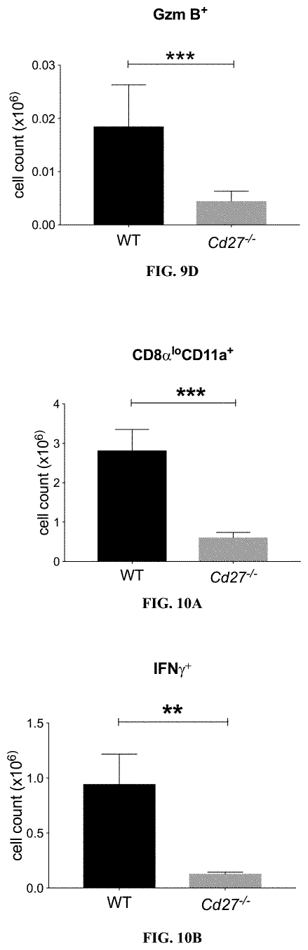

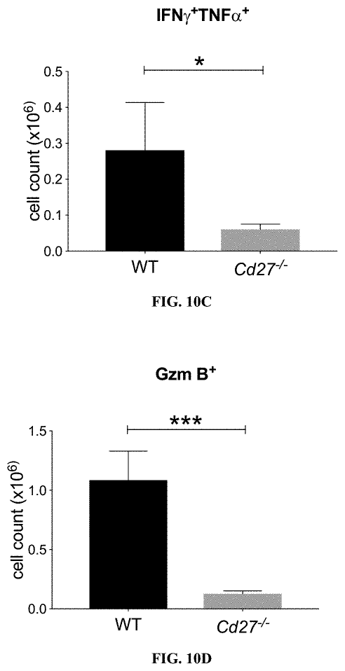

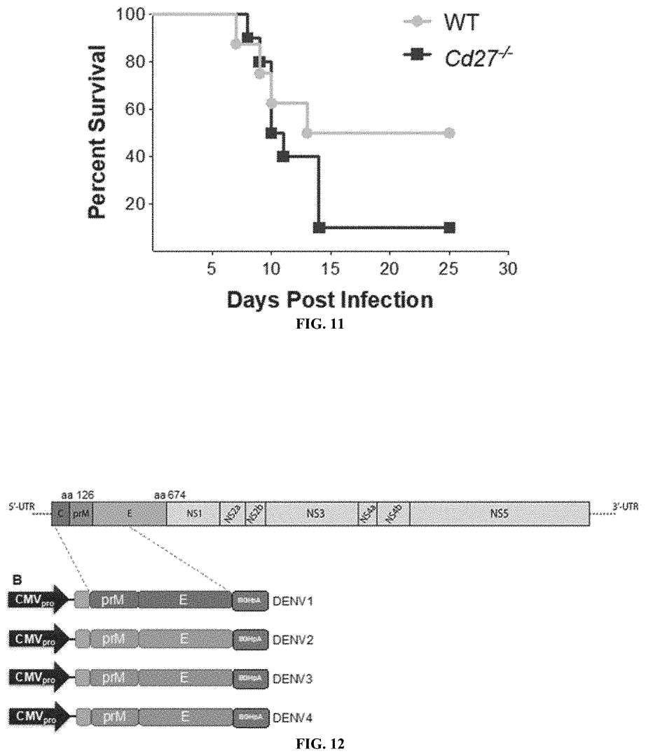

Studies with viruses such as vaccinia virus (VACV) and murine cytomegalovirus (MCMV) have demonstrated that several members of the TNF receptor superfamily, including CD27, can drive the generation of CD8 and CD4 T cell effector and memory cells, although the contribution to the responses can vary depending on the virus.⁵²⁻⁵⁴ Applicants have investigated the role of CD27 in modulating the anti-DENV CD8 T cell response and mediating protection against DENV using Cd27^{-/-} C57BL/6 mice and a clinical isolate of DENV2 (strain JHA1; Brazilian 2011 isolate). It was shown that DENV2 JHA1 infected and caused disease in BALB/c mice following an intracranial route of infection⁵⁵. For the present studies with Cd27^{-/-} mice, Applicants used this DENV2 strain to develop a WT C57BL/6 mouse model of DENV2 infection and disease upon subcutaneous route of infection (i.e. human relevant route of DENV infection). In preliminary experiments, Applicants observed that the total number of virus-specific CD8 T cells (i.e., CD8α^{lo}CD11a^{hi} cells that had been characterized as antigen-specific cells in various infection models^{56, 57}) and the numbers of immunodominant epitope-specific CD8 T cells expressing IFNγ, IFNγ and TNF, or Gzm B were significantly decreased in Cd27^{-/-} mice both at day 7 following primary DENV2 infection (FIGS. 9A-9D) and day 3 after secondary DENV 2 challenge (FIG. 10A-10D). These results demonstrate that CD27 plays a critical role in driving both the primary effector and secondary recall CD8 T cell responses during DENV infection in mice. This is similar to murine cytomegalovirus infection where the accumulation of both primary effector and memory CD8 T cell responses was reduced in the absence of the CD27-CD70 interactions⁵⁸. Additionally, a significantly greater percentage of Cd27^{-/-} mice succumbed to DENV2 challenge than WT mice (FIG. 11). These results suggest that T cell co-stimulation of CD27 can protect against DENV. Therefore, embodiments described herein manipulate the CD27-CD70 pathway to boost DENV and ZIKV vaccine-induced CD8 T cell immunity.

### Vaccines.

To create a tetravalent DENV-ZIKV vaccine, DNA plasmids encoding premembrane (prM) and E (or a consensus sequence thereof) of each DENV and ZIKV serotype are constructed. The sequences coding the prM and E proteins of each DENV serotype are cloned in the commercial vector pcDNA3, under the control of cytomegalovirus promoter (FIG. 12). Applicants have chosen prME-containing vaccines because co-expression of prM and E results in generation of secreted subviral particles⁶⁶⁻⁶⁸ which are more likely to induce potent nAbs that recognize complex quaternary epitopes than E alone⁶⁹⁻⁷¹. Sequences of recent DENV isolates from Brazil are selected to generate these vaccine candidates. WT mice in the C57BL/6 genetic background are immunized simultaneously with a mixture of all four prME DNA plasmids via an i.m. route using an electroporator.

Ab assays. To assess the immunogenicity of the ZIKV/DENV1-4 prME vaccine, Ab responses in the immunized mice are analyzed for levels of DENV-binding and nAb titers, as per standard approach in the field. DENV-binding IgG titers are measured by ELISA using plates coated with E protein from each virus or sucrose gradient-purified viral particles^{6, 32, 73}. In vitro neutralizing capacity of the Abs are measured by the standard Vero cell-based PRNT assay^{19, 20}.

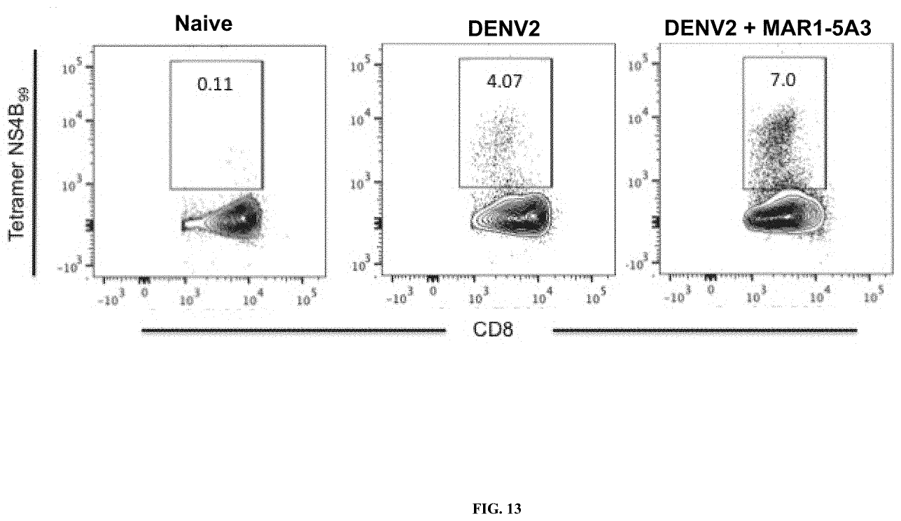

CD8 T cell assays. To monitor antigen-specific CD8 T cell responses in mice immunized with the ZIKA/DENV1-4 prME vaccine, peptide-MHC tetramer staining and ICS assays are used. Applicants identify K^{b}- and D^{b}-restricted epitopes in ZIKV and prM and E proteins of all four DENV serotypes^{30, 74, 75}. All immunodominant epitopes are tracked by ICS, and at least 1 by tetramer staining. Tetramers are obtained from the NIH tetramer facility (FIG. 13). To examine production of IFN-gamma, TNF, and IL-2 by epitope-specific CD8 T cells, cells are restimulated with peptide in vitro for ICS. CD8 T cells are examined for the expression of standard markers. In general, activated, pathogen-specific CD8 T cells are CD44^{hi} CD62^{lo} CD11α^{hi} CD8α^{lo}; effector CD8 T cells are CD107a⁺Gzm B⁺; short-lived effector cells (SLECs) are KLRG1⁺CD127⁻, memory precursor effector cells (MPECs) are KLRG1⁻CD127⁺; central memory T (T_{CM}) are CD62L⁺CD127⁺; and effector memory T (T_{EM}) are CD62L⁻CD127⁺⁷⁶⁻⁷⁸. As necessary, effector and memory CD8 T cells are also distinguished based on their expression of CX3CR1; T_{CM} are CX3CR1^{hi} and T_{EM} are CX3CR1^{hi} populations⁷⁹. As skin resident memory T cells (T_{RM}) offer superior protection to T_{CM} cells⁸⁰, and skin is a major target of DENV in humans and mouse models of DENV2 infection⁴⁴, T_{RM} cells are identified from their expression of CD69 and/or CD103, which mediate their retention within tissues⁸¹⁻⁸³. Intravascular and extravascular CD8 T cells are distinguished by i.v. injection of anti-CD8α 3 minutes prior to euthanasia. Extravascular cells are inaccessible to the anti-CD8α Ab, allowing tissue-resident cells to be identified by anti-CD8P staining⁸⁴.

To determine the optimal dose of immunization with ZIKV/DENV1-4 prME DNA plasmids for induction of CD8 T cell responses in WT mice, three different doses of ZIKV and/or DENV1-4 prME (0.5, 5, or 50 μg of each of the 4 plasmids mixed together) are tested. Groups of male and female mice at 5-6 weeks of age are immunized with one of two vaccine formulations: ZIKV/DENV1-4 prME or empty backbone plasmid. Mice vaccinated with a DNA plasmid expressing an irrelevant protein such as influenza hemagglutinin also serve as nonspecific controls. At 1, 3, and 6 months after immunization, CD8 T cell responses in the major lymphoid and non-lymphoid tissue targets of DENV and ZIKV (blood, spleen, lymph nodes, liver, and skin) are analyzed by tetramer staining and ICS. The anti-DENV and anti-ZIKV Ab responses are measured by ELISA and PRNT.

Without being bound to a particular theory, the medium and high doses (5 and 50 μg) of ZIKV/DENV1-4 prME vaccine elicit robust CD8 T cell (polyfunctional memory cells expressing multiple cytokines and Gzm B) and Ab responses (high ELISA-binding Ab titer and high nAb titer to each of the four DENV serotypes). Studies with other viruses, such as vaccinia virus, have demonstrated that the level of CD8 T cell immunity depends on viral virulence⁵⁴. Accordingly, the highest ZIKV/DENV1-4 prME dose (translating to a greater antigenic load over time) induces more memory CD8 T cells than lower doses of the vaccine. Tetramer staining and ICS at 1, 3, and 6 months after immunization defines the precise quantity, quality, location, and persistence of ZIKV/DENV1-4 prME-specific CD8 T cell responses in WT C57BL/6 mice. As previous DENV exposure or vaccination with a live attenuated vaccine focuses the CD8 T cell response on conserved/cross-reactive epitopes^{75, 85-87} Applicants observed immunodominance of conserved/cross-reactive epitopes. A complete set of H-2^{b}-restricted, cross-reactive CD8 T cell epitopes derived from each DENV serotype are identified, as DENV2-elicited, cross-reactive epitopes recognized by CD8 T cells in H-2^{b} mice with ZIKV infection³⁰ and HLA transgenic mice with DENV3 infection³⁴ were defined.

To determine the impact of the number of immunizations on levels of memory CD8 T cell responses, WT mice are immunized with ZIKV/DENV1-4 prME 1, 2, or 3 times with a 30-day interval between immunizations. The quantity, quality, and persistence of antigen-specific CD8 T cell responses on 1, 3, and 6 months after the last immunization are analyzed via tetramer staining and ICS. The optimal ZIKV/DENV1-4 prME dose was chosen based on results obtained from studies described above. Without being bound to a particular theory, mice with 3 immunizations have the highest levels of ZIKV/DENV1-4 prME-specific memory CD8 T cell responses. As repeated antigen exposure favors development of T_{EM} cells⁶¹⁻⁶⁴, the T_{EM} to T_{CM} ratio increases proportionally with additional immunization. The number of ZIKV/DENV1-4 prME-specific CD8 T_{RM} cells in the liver and skin are highest in mice with three immunizations.

prME expression was assessed by Western blotting of transiently transfected 293 T cell lysates. Additionally, secretion of subviral particles was determined by electron microscopic analysis of subviral particles that are isolated from culture supernatants of the transfected 293 T cells, sucrose gradient-purified, and then negative-stained. In certain alternative embodiments, the ZIKV/DENV prM signal sequence can be swapped with the analogous region of Japanese encephalitis virus (JEV)⁸⁹, or the signal peptide sequence of the human tissue plasminogen activator (t-PA)^{20, 21} to improve expression. In still other embodiments, the stem and transmembrane regions of DENV E can be substituted with corresponding JEV sequences⁹⁰ to improve subviral particle secretion. In still other embodiments, the dose of each plasmid relative to other three plasmids is modified to achieve balanced CD8 T cell responses against all four DENV serotypes.

In addition to the dose and frequency, a key parameter of immunization regimens is the interval between immunizations. Applicants compare 9 versus 30 days to determine the optimal immunization interval for the induction of ZIKV/DENV1-4 prME-specific CD8 T cell responses. In certain embodiments, two different immunization regimens induce similar levels of CD8 T cell responses; additional parameters related to the quality of CD8 T cell response are assessed. In particular, T cell avidity is measured by performing IFNγ ICS and tetramer straining with graded concentrations of DENV and/or ZIKV peptide. Additionally, the cytotoxic activity of DENV1-4 prME-specific or ZIKV-specific CD8 T cells against peptide-pulsed target cells is assessed via in vivo cytotoxicity assay. DENV1-4 prME-specific or ZIKV-specific CD8 T cell responses in other major targets of DENV and/or ZIKV, such as the intestine and kidney, are examined to see whether a particular immunization protocol influences the generation of CD8 T cell responses in a tissue-dependent manner.

Targeting CD27-CD70 interactions.

The TNF receptor superfamily members play an important role in modulating T cell effector and memory responses, and engagement of T cell costimulatory members of this superfamily improves antiviral CD8 T cell responses.⁵²⁻⁵⁴ Applicants show that CD27-deficient mice have decreased DENV2-specific primary effector and secondary recall CD8 T cell responses relative to WT mice, indicating a role for this TNF receptor superfamily member in regulating the anti-DENV2 CD8 T cell response (FIGS. 9A-9D and 10A-10D). Without being bound to a particular theory, targeting the CD27-CD70 pathway will boost ZIKV/DENV1-4 prME vaccine-induced CD8 T cell responses.

To boost ZIKV/DENV1-4 prME vaccine-induced CD8 T cell responses, the key T cell costimulatory molecule CD27, which is expressed on the surface of CD8 T cells, is stimulated. A DNA plasmid is designed to express soluble CD70 (which interacts with CD27), as the soluble recombinant CD70 mimics the function of membrane-bound CD70 on antigen-presenting cells⁹². WT mice are immunized with a mixture of 5 DNA plasmids (DENV1-4 prME and mouse CD70 or control (empty backbone) plasmid). Vaccinated mice are analyzed for CD8 T cell responses at 1, 3, and 6 months after the last immunization. Without being bound to a particular theory, CD27 engagement by soluble CD70 to increase the number of ZIKV and/or DENV-specific CD8 T cell memory cells.

Testing a pentavalent vaccine that includes ZIKV/DENV1-4 prME and consensus NS3. NS proteins, in particular NS3, NS4B, and NS5, are major targets of ZIKV and DENV-specific T cell responses in humans and mouse models^{27, 34, 45, 47, 93} Without being bound to a particular theory, ZIKV/DENV1-4 prME expressing a consensus NS3 protein induces a greater antigen-specific CD8 T cell and B cell responses than ZIKV/DENV1-4 prME alone.

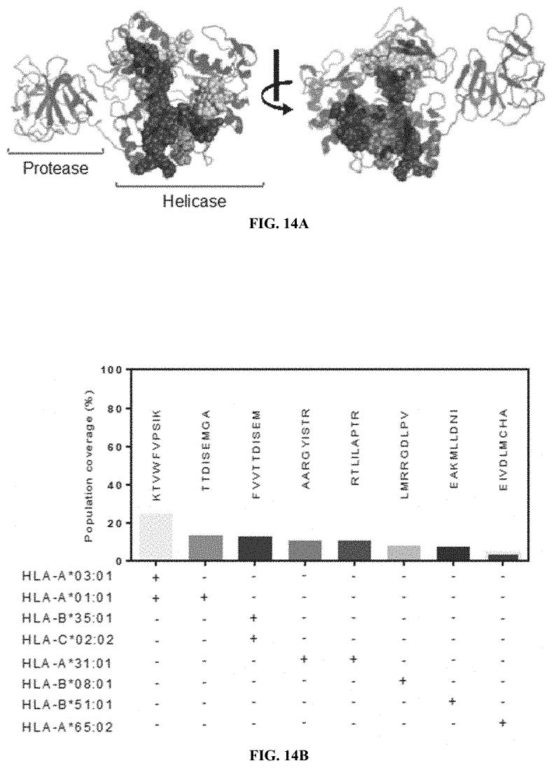

A DNA plasmid encoding a consensus sequence of NS3 from the ZIKV and 4 DENV serotypes is created (cNS3) (FIGS. 14A-14B). To design a cNS3 sequence, Applicants built a database of NS3 amino acid sequences from ZIKV and DENV1-4 isolates representing different years (1989 to 2018) and geographic regions of the world (Central and South America, Southeast Asia, and Africa). Fully annotated ZIKV and DENV NS3 sequences that were devoid of undefined amino acids were retrieved from the National Center for Biotechnology Information (NCBI). A consensus sequence among these ZIKV and DENV isolates is designed based on results obtained from multiple alignment of the different NS3 sequences using ClustalW method⁹⁴. The cNS3 amino acid sequence is used to compute a 3D model using the I-TASSER modeling method. The best computed model is subjected to TM-align structural alignment program to match the first I-TASSER model to all structures in the PDB library (Protein Data Bank—http://www.rcsb.org). The top 1 model is also used to determine epitope distribution in protein domains. Epitopes within the cNS3 protein that are restricted by commonly occurring HLA alleles in Brazil are predicted using the IEBD recommended method. The predicted epitopes from cNS3 are ranked by their percentile rank value and those with a percentile rank ≤1 are selected. Only predicted epitopes with a percentile rank <1 and are 100% conserved among all sequences of DENV1-4 are shown in FIGS. 14A-14B.

The cNS3-derived epitopes recognized by CD8 T cells from WT C57BL/6 mice are identified by using the IEDB prediction method and screening the predicted epitopes via IFNgamma-ELISpot analysis^{30, 74, 75}; overlapping peptides covering the cNS3 protein are tested. The optimized ZIKV/DENV1-4 prME immunization protocol described herein is used to compare antigen-specific CD8 T cell responses induced by the tetravalent ZIKV/DENV1-4 prME vaccine versus the pentavalent ZIKV/DENV1-4 prME plus cNS3. WT mice are immunized with each vaccine formulation, followed by analysis of antigen-specific CD8 T cell responses at 1, 3, and 6 months after the last immunization via ICS and tetramer staining. Non-vaccinated control are injected with DNA plasmids containing the empty backbone.

Without being bound to a particular theory, vaccination with ZIKV/DENV1-4 prME plus cNS3 elicits more robust antigen-specific CD8 T cell responses than ZIKV/DENV1-4 prME alone, in terms of magnitude, breadth, and persistence of the CD8 T cell response. The general features of the antigen-specific CD8 T cell responses, such as polyfunctionality, ratio between T_{EM} to T_{CM}, and anatomic location/T_{RM} numbers, is between the tetravalent and pentavalent vaccine candidates. Further embodiments combine the cNS3 DNA plasmid with T cell costimulatory receptor targeting (by way of example, and not by way of limitation, a DNA plasmid encoding both cNS3 and CD70).

In certain other embodiments, other TNF receptor superfamily members that are known to regulate CD8 T cell responses (such as OX40 and 4-1BB) are targeted⁵²⁻⁵⁴. Molecules like CD27 can be expressed on both activated T cells and NK cells. In still other embodiments, DNA plasmids encoding consensus NS4B and NS5 sequences from ZIKV and/or DENV1-4 are used either individually or together with the cNS3. In alternative embodiments describe the use of DNA plasmids encoding small domains of the NS protein from ZIKV and each of the four DENV serotypes.

Effect of ZIKV/DENV1-4 prME vaccine dose and number of immunizations.

Without being bound to a particular theory, the immunization protocol for the induction of the highest frequency of CD8 memory T cells with a polyfunctional phenotype (e.g., IFNγTNF+CD107a+) at 6 months after immunization provides superior protection against ZIKV/DENV challenge in mice.

To assess protection conferred by each of immunization protocol, the following 2 types of viral challenge experiments are performed:

(i) Immunization and challenge of WT mice. Groups of WT mice at 5-6 weeks of age are immunized and then challenged with each ZIKV and/or DENV serotype. Control animals include those vaccinated with plasmids that are empty or express influenza hemagglutinin protein. The immunized mice are treated with anti-Ifnar1 Ab (1 mg MAR1-5A3 via i.p. route) one day before s.c. challenge with the highest practical dose of DENV1 West Pacific 74 (1974 Pacific Island of Nauru isolate), DENV2 JHA1 (2011 Brazilian isolate)⁵⁵, DENV3 C0360/94 (1994 Thailand isolate)⁴⁰, and DENV4 703-4 (1994 Thailand isolate)⁹⁵. At day 3 after ZIKV and/or DENV challenge of the immunized mice, levels of viral RNA and infectious viral particles in tissues (serum, spleen, liver, skin, and brain) are measured by qRT-PCR and focus-forming assay (FFA), respectively. To assess the duration of vaccine-induced protection (i.e., to determine the longest-lived protection), mice are challenged on 1, 3, or 6 months after the last immunization. To determine whether CD8 T cells are required for the vaccine-induced protection, the immunized WT mice are depleted of CD8 T cells just prior to ZIKV/DENV challenge via i.v. injection of anti-mouse CD8 Ab or isotype control Ab as previously described^{27, 31, 74} CD8 T cell depletion in tissues is assessed via flow cytometry. Viral burden in tissues of CD8 T cell-depleted versus-undepleted immunized mice is compared at day 3 after viral challenge by qRT-PCR and FFA.

(ii) Adoptive transfer of T cells from immunized WT mice into congenic Ifnar1^{-/-} mice. WT mice are immunized, and CD8 T cells from the immunized mice are isolated on 1, 3, or 6 months after the last immunization. CD8 T cells are purified from spleen and lymph node cell suspensions using magnetic beads (Miltenyi Biotec), and then graded numbers (10⁷, 10⁶, and 10⁵) of the isolated CD8 T cells are transferred i.v. into naïve Ifnar1^{-/-} mice. One day after the adoptive transfer, the recipient mice are challenged via a s.c. route with a lethal dose of DENV2 JHA1, DENV3 C0360/94, or DENV4 703-4 or highest practical dose of DENV1 West Pacific 74.

Without being bound to a particular theory, the immunization protocol of 50 μg ZIKV/DENV1-4 prME and 3 rounds of immunization elicits the highest frequency of antigen-specific, memory CD8 T cells with a polyfunctional phenotype at 6 months after and provides the best protection. WT mice have decreased levels of viral RNA and infectious virus upon viral challenge and exhibit the longest-lived protection, as compared to mice immunized with suboptimal protocols for the induction of CD8 T cell responses. CD8 T cell depletion validates the importance of CD8 T cells in mediating vaccine-induced protection, particularly when the vaccinated mice are challenged at a late time point (6 months) after immunization. In agreement with these anticipated results from WT mouse experiments, Ifnar1^{-/-} mice that are adoptively transferred with CD8 T cells from the WT group immunized with the optimal protocol exhibit improved survival and decreased viral burden relative to Ifnar1^{-/-} recipients that are adoptively transferred with equal numbers of naïve T cells or T cells from WT mice immunized with suboptimal protocols. The level of protection conferred by the adoptively transferred CD8 T cells positively correlates with the number of transferred cells.

Applicants evaluate the protective efficacy of these two new vaccine modalities that are expected to induce higher magnitude of ZIKV- and DENV-specific CD8 T cell and B cell responses relative to ZIKV/DENV1-4 prME. Without being bound to a particular theory, new vaccine modalities described herein provide a more robust and longer-lived protection against ZIKV/DENV challenge in mice than ZIKV/DENV1-4 prME alone.

The efficacy of protection mediated by ZIKV/DENV1-4 prME plus mouse CD70 or cNS3 is assessed by performing the two different types of challenge experiments described above. Appropriate control animals are vaccinated with plasmids that are empty or express influenza hemagglutinin protein.

It is important to assess how the different vaccine modalities alter the anti-DENV and or anti-ZIKV Ab response in vivo. The capacity of the vaccine-induced Ab responses to mediate protection or ADE in mice is assessed. Graded amounts of immune sera harvested from immunized WT mice are passively transferred into naïve Ifnar1^{-/-} mice, followed by challenge of the passively transferred recipient mice with ZIKV/DENV. On day 3 after ZIKV/DENV challenge, mice are monitored for survival and analyzed for viral burden in tissues. The quality of immune sera from the immunized mice in terms of ELISA binding and PRNT titers is correlated with protective versus pathogenic effect in mice.

Mice are treated with Abs that deplete CD4 T cells and/or B cells prior to each immunization. Alternatively, gene-deficient mice lacking B cells or CD4 T cells are used. In case 2 different immunization protocols or vaccine modalities induce similar levels of protection, other key features of DENV infection besides survival and tissue viral burden (e.g., cytokine storm, vascular leakage, low platelet count, and hemorrhage) are examined. Additional time points after viral challenge besides day 3 (e.g., days 1, 5, and 7 after viral challenge) may be necessary to observe differences between two immunization protocols/vaccine modalities. Additional non-lymphoid target tissues such as the intestine and kidney are analyzed for viral burden, and the magnitude and phenotype of CD8 T_{RM} populations in these tissues as correlated with viral burden is assessed.

### Example 2: Evaluation of Immunogenicity and Protective Efficacy of Pan-Flavivirus Vaccine

Interplay between pre-existing Ab and T cell responses can determine the outcome of DENV/ZIKV infections. In particular, studies using mouse models that recapitulate key aspects of severe dengue such as plasma leakage and cytokine storm in different vaccination and maternal Ab-mediated ADE settings have demonstrated that DENV Abs do not protect against different DENV strains of the same serotype and that CD8 T cells can actually prevent DENV-ADE. A vaccine that elicits both Ab and CD8 T cell responses may also be suitable for infants, as maternal DENV Abs seem to interfere less with induction of vaccine-induced CD8 T cell responses than with Ab responses. Although infants and young children represent key target populations for DENV vaccination, Dengvaxia® cannot be used in children who are younger than 9 years of age or are DENV-naïve. The next two leading DENV vaccine candidates, created by NIH and Takeda, were also not designed to generate optimal CD8 T cell responses. The NIH vaccine lacks the NS proteins (i.e. major CD8 T cell target proteins) of DENV2, a serotype that is frequently associated with severe dengue disease manifestations and is also most common breakthrough virus for people immunized with Dengvaxia®. The Takeda vaccine lacks the NS proteins of DENV1, DENV3, and DENV4, and all DENV serotypes can cause severe dengue. Thus, a vaccine that elicits both Ab and T cell responses to the E and NS proteins from DENV1-4 and ZIKV could reduce the global impact of DENV and ZIKV. A pan-flaviviral vaccine strategy that elicits robust Ab and T cell responses against the 4 DENV serotypes and ZIKV is tested for providing protection against all five viruses in mice.

Determination Whether Vaccination with a Pan-Flavivirus Vaccine Containing prME or NS3 of Each of the Five Viruses (4 DENV Serotypes and ZIKV) Generate Robust Ab and T Cell Responses Against Each Virus in WT Mice

The immunogenicity of a decavalent vaccine in WT C57BL/6 mice is evaluated. DENV replicons encoding prME with the mutant FL epitope in E-DII or NS3 is generated using contemporaneous DENV sequences. Mice are immunized with a combination of 10 replicons twice (10 μg each, i.m. route, 28 days apart), and sacrificed 28 days after the boost to assess vaccine-induced Ab and T cell responses in the serum and spleen, respectively. Although mice have been injected with 90 μg of mRNA-LNP formulation, the replicon dose is reduced to 5 μg each if toxicity is observed with 10 μg. Control mice are injected with saline or a replicon encoding influenza hemagglutinin.

CD8 T cell assays. To monitor vaccine-elicited CD8 T cell responses, expression of granzyme B, IFNg, and TNF by antigen-specific CD8 T cells is evaluated in splenocytes restimulated with peptides, followed by ICS. ZIKV- and DENV2-derived epitopes recognized by CD8 T cells in H-2^{b} mice and DENV1-, DENV3-, and DENV4-derived epitopes restricted by H-2^{b} CD8 T cells are identified as was determined for ZIKV and DENV2. ICS is performed with each identified peptide in case the vaccination strategy impacts the immunodominance patterns. Peptide-specific CD8 T cells are examined for the expression of markers associated with various differentiation states. Short-lived effector cells (SLECs) that may not persist and become memory cells have been described as KLRG1⁻CD127⁺; (among other markers), memory precursor effector cells (MPECs) as KLRG1⁻CD127⁺; central memory T cells (TCM) as CD62L⁺CD127⁺; and effector memory T cells (TEM) as CD62L⁻CD127⁺. To further define distinct CD8 T cell populations, CD8 T cells are also subsetted into CX3CR1⁻ (T_{CM}), CX3CR1^{hi} (T_{EM}), and CX3CR1^{int} (peripheral memory T; T_{PM}) cells.

CD4 T cell assays. To assess vaccine-elicited total CD4 T cell responses in the spleen, the surrogate-activation-marker approach is applied that is based on the expression of the integrins CD49d and CD11a and has been extensively characterized in various infection models, including viruses, to define antigen-specific CD4 T cells as CD49d^{hi}CD11a^{hi}CD3⁺CD4⁺ cells by flow cytometry. Each virus-specific CD4 T cell response is measured in the spleen by ICS. Based on H-2b-restricted DENV2- and ZIKV-derived epitopes recognized by CD4 T cells DENV1-, DENV3-, and DENV4-derived epitopes restricted by H-2^{b} CD4 T cells are identified. CD4 T cells are analyzed for the expression of CD40L, CD44, CD62L, CD25, CD127, Foxp3 (regulatory T cell marker), CD107a and granzyme B (cytotoxicity marker), and intracellular cytokines and transcription factors that define the following Th subsets: IFNg, TNF, IL-2, T-bet (Th1), IL-10, IL-4, IL-5, GATA3 (Th2), IL-17, RORC (Th17), and IL-21, Bcl6 (Tfh). GC and non-GC Tfh are identified based on the expression of CXCR5 and PD-1, and Tfh functions are assessed by measuring the frequency of non-GC (CD19⁺CD20⁺B220⁺) and GC (CD19⁺B220⁺GL7⁺Fas⁺) B cells and plasma cells (CD19⁺B220⁺CD138⁺).

Ab assays. To assess the quantity and quality of the Ab response to the vaccine, sera are analyzed for levels of virus-specific IgG by ELISA using plates coated with E protein from each virus or sucrose gradient-purified viral particles. Serum avidity is measured by urea ELISA where strength of serum binding to the antigen is measured by the susceptibility of binding to disruption by 7, 8, or 9 M urea. In vitro neutralizing capacity of the Abs is measured by the standard U937-DC-SIGN/FACS assay.

The decavalent pan-flavivirus vaccine elicits robust CD8 T cell (polyfunctional memory cells expressing multiple cytokines and the cytotoxic granule protein granzyme B; >>10⁶ immunodominant epitope-specific cells), CD4 T cell (antigen-experienced Tfh and polyfunctional Th1 cells expressing multiple cytokines and granzyme B; >>10⁵ immunodominant epitope-specific cells), and Ab responses (high ELISA-binding Ab titer and avidity and high nAb titer; NT50 values of >>1/1000) to each of the five viruses. The precise Ab titers and T cell numbers that correlate with protection are determined. As prior DENV exposure or vaccination with a live attenuated tetravalent DENV vaccine focuses the CD8 T cell response on conserved/cross-reactive epitopes, immunodominance of conserved/cross-reactive epitopes is sometimes observed. If necessary, a complete set of H-2^{b}-restricted, cross-reactive CD8 and CD4 T cell epitopes derived from each virus is identified, as DENV2-elicited, cross-reactive epitopes recognized by CD8 T cells in H-2^{b} mice with ZIKV infection and HLA transgenic mice with DENV3 infection were previously determined.

Determination of Protection of the Pan-Flavivirus Vaccine Against Each DENV Serotype and ZIKV in WT Mice

The short-term (1 month-long) protective efficacy of the pan-flavivirus vaccine is evaluated (i.e. the vaccination regimen described herein). WT C57BL/6 mice are immunized as described above, followed by treatment with an Ifnar1-blocking Ab (1 mg MAR1-5A3 via i.p. route) on day 56 (28 days after the boost), and challenge with each DENV serotype or ZIKV on day 57 (29 days after the boost). On day 3 after viral challenge, tissues are harvested to assess viral burden.

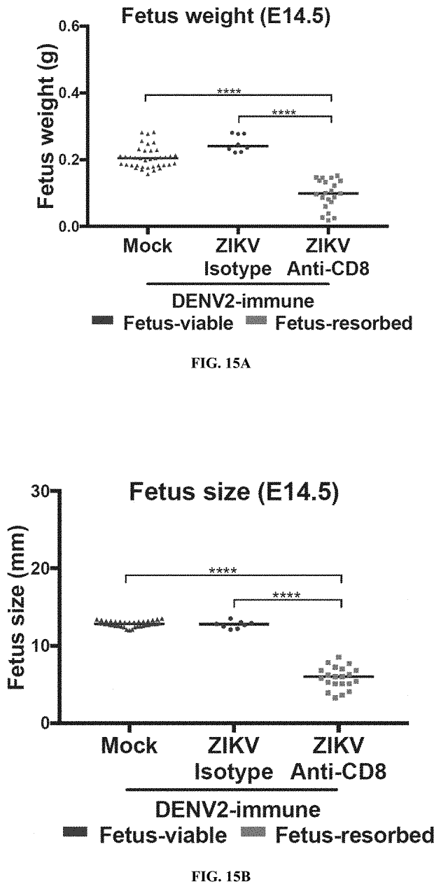

Viral challenge. The following DENV strains are used to challenge vaccinated mice: DENV1 West Pacific 74 (1974 Pacific Island of Nauru isolate), DENV2 S221 (mouse-adapted in our laboratory using Taiwanese strain PL046), DENV3 C0360/94 (1994 Thailand isolate), and DENV4 703-4 (1994 Thailand isolate), based on published and unpublished data showing that these strains replicate in Ifnar1−^{/-} or anti-Ifnar1 Ab-treated WT mice. ZIKV strain SD001 (2016 clinical isolate from a San Diego traveler to Venezuela; Asian lineage), representing a Brazil 2014-2016 epidemic strain, are used. Viruses are propagated in C6/36 Aedes albopictus cells, and viral titers will be measured by BHK-21 cell-based focus-forming assay (FFA). Virgin male and female WT mice that have been immunized with the pan-flavivirus vaccine are challenged with the highest practical dose (at least 10⁶ FFU) of DENV1, DENV2, DENV3, DENV4, or ZIKV via a subcutaneous (s.c.) route. As adult 14- to 16-week-old WT mice do not exhibit overt signs of clinical disease following DENV/ZIKV infection, protection is assessed based on viral burden in key target organs of each virus. At day 3 after viral challenge, mice are sacrificed, and levels of viral RNA and infectious particles in tissues (serum, spleen, liver, draining lymph nodes, intestine, brain, eyes, testes, and female reproductive organ) are measured via qRT-PCR and FFA, respectively. The day 3 post-infection (p.i.) time point allows focus on the vaccine-induced immune response since the primary T cell response to DENV or ZIKV infection in mice is not detectable until day 5 p.i. As congenital Zika syndrome is a key feature of ZIKV pathogenesis, vaccine efficacy against ZIKV infection is evaluated in pregnant mice. A cross-protective role for DENV2-elicited CD8 T cells against subsequent ZIKV infection in syngeneically pregnant Ifnar1−^{/-} and anti-Ifnar1 Ab-treated WT mice was established using the more human relevant allogeneically pregnant mice (FIGS. 15A-15B). WT C57BL/6 virgin females are thus immunized and then mated with naïve WT BALB/c sires to generate allogeneically pregnant, vaccinated mice, followed by injection of pregnant mice at E6.5 with anti-Ifnar1 Ab and inoculation at E7.5 with ZIKV or mock-infected with PBS/10% FBS. At E14.5 (7 days after ZIKV challenge), fetal weights and sizes are measured using an analytical balance and digital caliper, respectively, and viral burden in maternal tissues (serum, brain, liver, and lymph nodes), placenta, and fetal tissues (head and body) are quantified by qRT-PCR.

Vaccinated mice exhibit lower viral burden than naïve mice following each viral challenge. In vaccinated pregnant mice with ZIKV infection, the placenta and fetal tissues contain lower levels of ZIKV RNA and fetal weights and sizes are higher relative to those in naive pregnant mice with ZIKV infection. Fetal weights and sizes in vaccinated pregnant mice with ZIKV infection are normal and thus similar to those in mock-infected pregnant mice.

Optimization of the pan-flavivirus vaccine. First, the immunization regimen is optimized, including each replicon dose and the frequency and interval of the vaccine administration.

Second, bigenic replicons encoding both prME and NS3 of each virus are tested. Bigenic replicons not only allow immunization of mice with a combination of 5 (instead of 10) replicons, but expression of both prME and NS3 in the same replicon can result in increased protection.

Third, a consensus NS3 replicon that contains conserved regions in all 5 viruses is tested. To create this consensus replicon, NS3 sequence of one of the DENV serotypes is modified to encode regions that are conserved in NS3 proteins from other three DENV serotypes and ZIKV, which share 66-67% amino acid identity. If the consensus vaccine does not protect equally against each of the five viruses, a new composite NS3 gene is synthesized that contains both cross-reactive and virus-specific NS3 epitopes restricted by multiple HLA alleles and recognized by most humans in the world. Epitopes from another T cell target protein such as NS5 are also added in some cases. The immunogenicity and efficacy of this novel composite NS3 gene is then tested using HLA transgenic mice. HLA-transgenic mice have been used to investigate the anti-DENV and anti-ZIKV T cell responses restricted by human MHC class I alleles A*0101, A*0201, A*1101, and B*0702 (which cover four different HLA supertypes and cover ˜90% of the population worldwide 110) and MHC class II allele DRB1*010110, 12, 83. Overall, either the consensus or composite vaccine replaces five virus-specific NS3 replicons, allowing use of a hexavalent or trivalent instead of decavalent or pentavalent formulation.

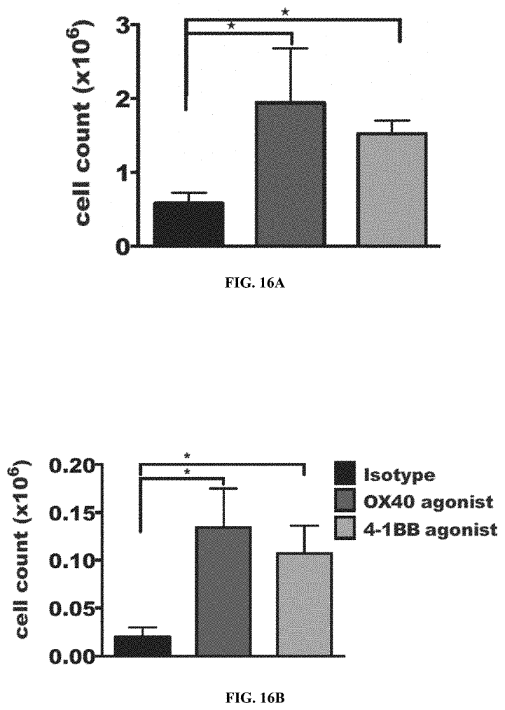

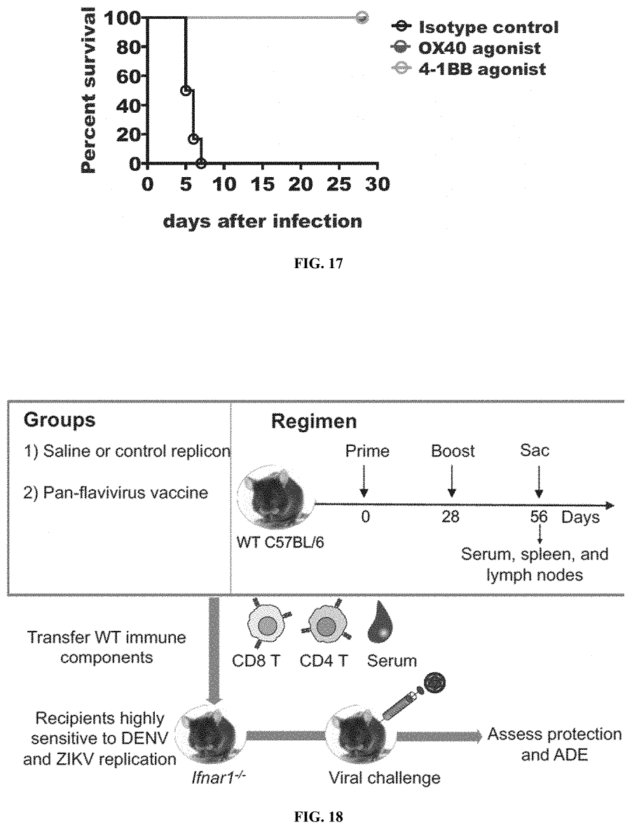

Fourth, T cell costimulatory molecules are manipulated, such as the TNF receptor superfamily members OX40 and 4-1BB, that are present on activated T cells. Based on preliminary results suggesting that stimulation OX40 and 4-1BB via agonistic Abs promotes DENV2-specific T cell responses and protects against lethal DENV2 challenge in Ifnar1−^{/-} mice (FIGS. 16A-16B and FIG. 17), replicons encoding OX40 ligand and 4-1BB ligand are created.

### Example 3: Assessing Durability and Immune Mechanisms Underlying the Pan-Flavivirus Vaccine-Induced Protective Immunity and ADE

Based on the general paradigm that Abs prevent viral infection while effector T cells control replication once infection has been established, a vaccine is developed that elicits both humoral and cellular immunity against the four DENV serotypes and ZIKV. This approach generates a safe and effective pan-flavivirus vaccine because a long-lived CD8 T cell response will destroy virus-infected cells and prevent ADE as Ab levels decline. Eliciting the effector functions of both Abs and T cells may also be key for protecting against fetal ZIKV infection. Indeed, the Ab-centric ZIKV vaccine candidates tested to date produce only partial protection against fetal ZIKV infection, and CD8 T cells have been shown to protect against ZIKV transplacental transmission. Thus, the vaccine strategy will confer durable protection against all five viruses.

Vaccination with the Pan-Flavivirus Vaccine Induces Long-Lived CD8 T Cell-Mediated Protection in Mice

The contribution of CD8 T cells to the protection induced by the pan-flavivirus vaccine is evaluated by depleting CD8 T cells in vaccinated WT mice; and confirmed by adoptive transfer of CD8 T cells from vaccinated WT C57BL/6 mice into congenic Ifnar1−^{/-} recipient mice. The adoptive transfer approach allows assessment of vaccine-induced immune response produced within a normal environment in WT mice, and to evaluate the contribution of CD8 T cells vs. CD4 T cells vs. Abs separately in vaccine-induced protection (FIG. 18). Moreover, Ifnar1−^{/-} recipient mice are highly sensitive to infection with both DENV and ZIKV, and thus represent a very stringent challenge system. To define the role of CD8 T cells, tissue viral burden of vaccinated WT mice with or without CD8 T cell depletion and both survival and tissue viral burden of Ifnar1−^{/-} T cell recipient mice and control mice after challenge with each of the 5 viruses is compared. The durability of vaccine-mediated protection is assessed by challenging mice at 1, 3, and 6 months after immunization.

CD8 T cell depletion and adoptive transfer of CD8 T cells. To evaluate whether CD8 T cells are required for the pan-flavivirus vaccine-mediated protection, WT mice that have been vaccinated for 1, 3, and 6 months are i.v. injected with anti-mouse CD8 Ab or isotype control Ab. CD8 T cell depletion in tissues is monitored by flow cytometry. Mice are sacrificed at day 3 after challenge with the highest practical dose of each virus to measure viral burden in tissues via qRT-PCR and FFA. To evaluate protective capacity of CD8 T cells by themselves, spleens and lymph nodes of WT mice immunized with the pan-flavivirus vaccine or saline/irrelevant replicon (controls) are harvested at 1, 3, and 6 months after the last immunization. The magnitude and quality of the vaccine-induced CD8 T cell response in the spleen and lymph nodes are assessed via ICS as described above. For adoptive transfer, CD8 T cells are isolated by magnetic bead negative selection, followed by i.v. transfer of graded numbers (10⁷, 10⁶, and 10⁵) of CD8 T cells into naïve Ifnar1^{-/-} mice 1 day before viral challenge. Control Ifnar1^{-/-} recipients are infused with naïve T cells from mock-vaccinated WT mice. Recipient mice are s.c. challenged with a lethal dose of DENV2 S221, DENV3 C0360/94, DENV4 703-4, or ZIKV SD001. As a DENV1 strain that can cause lethal disease in Ifnar1^{-/-} mice is as yet to be identified, the highest practical dose (10⁶ FFU) of DENV1 West Pacific 74 is used. Survival, weight loss, and clinical score are monitored for 30 days, and viral RNA and infectious virus levels in various tissues are quantified via qRT-PCR and FFA, respectively, on day 3 after viral challenge. Pregnant Ifnar1^{-/-} recipients are challenged with ZIKV and then evaluated for viral burden as described above.

CD8 T cell-depleted mice exhibit higher viral burden (and increased fetal ZIKV burden and abnormal fetal growth in pregnant mice) compared with their CD8 T cell intact counterparts. Adoptive transfer of CD8 T cells from WT mice immunized with the pan-flavivirus vaccine protect against all 5 viruses in Ifnar1^{-/-} mice, as measured by improved survival, weight loss, and clinical score and decreased viral burden relative to control mice (and reduced maternal and fetal ZIKV burden and fetal demise in pregnant mice). The durability of protection correlates with the magnitude and phenotype (including breadth and polyfunctionality) of the vaccine-elicited CD8 T cell responses at 1, 3, and 6 months after immunization.

Vaccination with the Pan-Flavivirus Vaccine Induces Long-Lived CD4 T Cell-Mediated Protection in Mice

To investigate the role of CD4 T cells in mediating the protection induced by the pan-flavivirus vaccine, the same T cell depletion and adoptive transfer approaches described above for analyzing the role of CD8 T cell responses in protection are used.

CD4 T cell depletion and adoptive transfer of CD4 T cells. Vaccinated WT mice are depleted of CD4 T cells. CD4 T cells from the vaccinated WT mice are isolated, defined via FACS and ICS as described above, and adoptively transferred into naïve Ifnar1^{-/-} mice as described above. CD4 T cell-depleted, vaccinated WT mice and adoptively transferred Ifnar1^{-/-} recipient mice are challenged with each virus and analyzed for clinical and virologic phenotype, as described above.

The pan-flavivirus vaccine elicits a high frequency of antigen-specific effector CD4 T cells with a polyfunctional Th1 phenotype that confer protection against all five viruses even 6 months after immunization. However, effector CD8 T cells are more important than effector CD4 T cells in directly inhibiting DENV and ZIKV replication and vaccine-induced protection may be similar in CD4 T cell-depleted and -sufficient mice. In this case, the adoptive transfer studies are focused on evaluating the ability of CD4 T cells to boost Ab responses, as the longevity of Tfh cells can be key to Ab persistence. Tfh responses are evaluated both by FACS and immunohistochemistry for the expression of peanut agglutinin (PNA) or GL7 (which marks GC B cells), and IgM or IgD (which stains for follicular B cells) in Ifnar1^{-/-} recipient mice following viral challenge.

Vaccination with the Pan-Flavivirus Vaccine Induces Long-Lived Ab-Mediated Protection in Mice

To examine protective vs. pathogenic Ab (ADE-mediating) responses induced by the pan-flavivirus vaccine, sera from vaccinated WT mice is harvested at 1, 3, and 6 months after immunization to perform passive transfer experiments (FIG. 18).

Passive transfer of Abs. Pooled sera (e.g., 2, 20, and 200 μl, representing different nAb titers) are transferred via an i.p. route into naïve Ifnar1^{-/-} mice one day before viral challenge. Recipient mice are s.c. challenged with a lethal/highest possible dose of each of the five viruses. Separate groups of recipient mice are challenged with a sub-lethal viral dose, which allows us to assess the ADE capacity of the immune sera. One group of control mice receives sera from mice immunized with replicons containing the E-DII FL epitope, while another receives sera from mice immunized with and irrelevant replicon or saline. For both sublethal and lethal/highest possible viral challenge dose studies, one group of mice will be monitored for weight loss, clinical score, and survival for 30 days, while a second group will be used to measure viral RNA and infectious virus levels in tissues on day 3 p.i. by qRT-PCR and FFA, respectively.

The anti-flaviviral Ab response can have three different effects: protection, enhancement, or negligible effect. The major features of the Ab response that determine the outcome of infection include epitope specificity/accessibility, concentration, and avidity. Neutralization versus enhancement is a function of the stoichiometry of Ab binding to the viral particle. Even “neutralizing” Abs neutralize only if present at sufficiently high concentration; if present at concentrations below the threshold required for neutralization, they may promote ADE or exert “negligible” effect. Accordingly, the following results are observed in Ifnar1^{-/-} recipients: A high amount/nAb titer (e.g., 200 μl) of immune sera protects against all 5 viruses whereas low doses (2 or 20 μl) of the immune serum should induces a neutral effect rather than ADE due to the absence of the immunodominant E-DII FL epitope that elicits cross-reactive enhancing Abs. In contrast, the immune sera from mice vaccinated with replicons containing the E-DII FL epitope mediates ADE.

### Example 4: Assess the Immune Response and Protection Induced by the Pan-Flavivirus Vaccine in Nonhuman Primates

Although mice are invaluable models for understanding the mechanisms underlying vaccine-mediate protection, nonhuman primates are the gold standard for predicting vaccine efficacy in humans. Therefore, the ability of the pan-flavivirus vaccine described herein to protect rhesus macaques (RM) from challenge with ZIKV and one DENV serotype, DENV2 is determined. DENV2 is selected over other DENV serotypes, because DENV2 is a dominant global serotype and is often associated with severe dengue, Dengvaxia® is least effective against DENV2, the next leading DENV vaccine candidate (i.e. the NIH vaccine) lacks DENV2 NS proteins, and DENV2 infection in RM is better characterized than other DENV serotypes. Although it would be informative to challenge animals with each of the five viruses, limiting the study to one DENV serotype and ZIKV reasonably balances the use of the limited RM resource. The pan-flavivirus vaccine that protects against all five viruses in the mouse studies induces robust Ab and T cell responses against all five viruses and confers protection against DENV2 and ZIKV in RM.

Nonhuman primate models of ZIKV and DENV infection. Nonhuman primates (NHP) are highly susceptible to ZIKV infection and infection of fetal RM results in a range of neurodevelopmental pathology that is consistent with human fetal ZIKV infection. A ZIKV vaginal transmission model in RM127 has shown that RM are infected after vaginal inoculation with a dose of ZIKV that is within the range found in human semen, further supporting the use of RM as an excellent animal model of human ZIKV infection. In comparison, NHP develop few clinical signs after experimental DENV infections. The level of viremia after experimental DENV infection in NHP is lower than in natural infection of humans, likely because DENV is unable to inhibit Ifnar1 signaling in NHP cells as efficiently as in human cells. In addition, NHP challenge protocols often involve DENV inoculation via s.c. or intradermal (i.d.) routes, which are thought to reflect a mosquito bite. Sustained levels of viremia accompanied by signs of hemorrhages have been reported for RM experimentally infected with DENV via an i.v. route, which may mimic direct injection into blood capillaries when a mosquito feeds on its host. The attenuated disease course following s.c. or i.d. inoculation may have overestimated vaccine efficacy in preclinical testing of DENV vaccines, including Dengvaxia®. Indeed, results of a recently published NHP study of Dengvaxia®, in which the vaccinated monkeys were i.v. challenged and assessed for viremia, were consistent with reported poor clinical vaccine efficacy of Dengvaxia® against DENV2. Thus, despite its limitation of not manifesting severe dengue disease, the NHP model can predict the efficacy of DENV vaccines in humans if an i.v. challenge system is used.

The criteria for selecting a vaccine formulation to test in NHP are: 1) degree of protection in mice, 2) nAb titers, 3) magnitude and phenotype of the T cell responses (polyfunctional>mono- or bi-functional) that correlate with protection against each of the five viruses in mice, 4) ability to prevent ADE, and 5) durability of vaccine induced immune responses. The formulation that provides the strongest T cell and nAb responses, and shows an ability to prevent ADE is selected.

The Pan-Flavivirus Vaccine Elicits Each of the Five Virus-Specific Ab and T Cell Responses in RM

To confirm the immunogenicity of the pan-flavivirus vaccine from Examples 2 and 3 in NHP, a group of 6 flavivirus-naïve RM (Group 1) is immunized twice (8 weeks apart) with 100 μg of each RNA replicon. This dose of replicon is based on a study demonstrating that RM immunized with 50 μg or 600 μg are protected from ZIKV challenge. Juvenile RM of both genders are used. Ab and T cell responses in blood are assessed the day of immunization, 7 and 14 days after each immunization, and 8 weeks after the last immunization.

Group 1 (N=6) is immunized with the pan-flavivirus vaccine, and blood collected for assessing anti-DENV and anti-ZIKV immunity.

Ab assays. Serum samples are used to measure ELISA-binding and nAb titers and avidity as described in Example 2.

T cell assays. Recent publications have characterized ZIKV-specific T cells in blood of ZIKV-infected RM using flow cytometry after in vitro stimulation of cells with peptide pools. To measure the frequency of DENV- and ZIKV-specific CD4 and CD8 T cells, peripheral blood mononuclear cells (PBMC) are stimulated with peptide pools (ZIKV/DENV1-4 NS3 and prME) for 6 hours and ICS is performed to measure cytokine expression (IFNg, IL-2, IL-17, and TNF) in CD8 and CD4 T cells. Phenotype staining of these T cells before and after stimulation focuses on markers of cytotoxic potential (CD107, perforin, or granzyme B), Tfh differentiation, and memory/effector function. The memory/effector phenotype of T cells will be defined based on CD28 and CD95 expression. Naïve T cells are CD28^{int/hi}CD95⁻; TCM cells are CD28^{hi}CD95^{hi}; and TEM cells are CD28⁻/CD95^{hi}. Tfh functions will be assessed by measuring the frequency of immature (CD3⁻CD10⁺CD20⁺) and mature (CD3⁻CD10⁻CD20⁺) B cells, plasmablasts (CD19⁺CD20⁻CD38⁺CD138⁺) and plasma cells (CD19⁺CD20⁻CD38⁺⁺ CD138).

The pan-flavivirus vaccine elicits robust CD8 and CD4 T cell and Ab responses (with similar phenotype/features as observed in mice) to each of the five viruses in RM. Vaccine-induced T cell responses are detectable 7 days after the first immunization and vaccine-induced Abs are detectable by 14 days post-vaccination, with both responses being rapidly boosted by the second immunization, persisting at high levels until challenge.

The Pan-Flavivirus Vaccine Affords Protection Against DENV2 and ZIKV in RM

To confirm the efficacy of the pan-flavivirus vaccine, 2 groups of 6 RM (Groups 2 and 4) are immunized with the pan-flavivirus vaccine and 2 groups will be immunized with an irrelevant RNA replicon (Groups 3 and 5). Flavivirus-naïve, juvenile (2- to 3-year-old) RM of both genders are immunized and boosted exactly as above. Each group of immunized animals will then be challenged with either DENV2 or ZIKV.

Group 2 (N=6) is immunized with the pan-flavivirus vaccine, then challenged s.c. with ZIKV.

Group 3 (N=6) is immunized with an irrelevant RNA replicon, then challenged s.c. with ZIKV.

Group 4 (N=6) is immunized with the pan-flavivirus vaccine, then challenged i.v. with DENV2.

Group 5 (N=6) is immunized with an irrelevant RNA replicon, then challenged i.v. with DENV2.

Viral challenge. The DENV2 strain16681 that reliably produces high level viremia after i.v. inoculation of macaques and ZIKV strain SD00113 are used. Vaccinated animals are inoculated with 10⁷ FFU of DENV2 via the i.v. and ZIKV via s.c. route. Protection is assessed by determining viral burden (viral RNA via qRT-PCR and infectious virus by FFA) in plasma collected at 2, 4, 6, 8, 10, 12, 14, 16 and 18 days after challenge. In addition, at day 21 after viral challenge, RM are sacrificed, and levels of viral RNA and infectious particles in tissues (plasma, spleen, liver, draining lymph nodes, intestine, brain, eyes, testes, and reproductive tract) are measured via qRT-PCR and FFA, respectively. The day 21 p.i. time point allows assessment the strength and quality of vaccine-induced anamnestic immune responses to DENV or ZIKV challenge in RM.

The vaccinated RM are completely protected from the ZIKV and DENV2 challenges, with very low or undetectable viral RNA in plasma that is associated with strong Ab and T cell responses before challenge. If some vaccinated RM become infected, they will have lower viral burden than naïve RM after viral challenge. The level of protection correlates with vaccine-induced Ab and T cell responses.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments are not meant to be construed in a limiting sense. Numerous variations, changes, and substitutions will now occur to those skilled in the art without departing from the invention. Furthermore, it shall be understood that all aspects of the invention are not limited to the specific depictions, configurations, or relative proportions set forth herein which depend on a variety of conditions and variables. It should be understood that various alternatives to the embodiments described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A composition comprising:
a) a protein or peptide, or variant, homologue, derivative, or subsequence thereof, that comprises, consists, or consists essentially of a flavivirus B cell epitope, or a nucleic acid molecule encoding the protein or peptide, or variant, homologue, derivative, or subsequence thereof, and
b) a protein or peptide, or variant, homologue, derivative, or subsequence thereof, that comprises, consists, or consists essentially of a flavivirus T cell epitope or a nucleic acid molecule encoding the protein or peptide, or variant, homologue, derivative, or subsequence thereof,
wherein the composition elicits, stimulates, induces, promotes, increases, or enhances an antibody response and a T cell response against two or more different serotypes of a flavivirus or two or more different species of flavivirus.

2. The composition of claim 1, wherein the composition elicits, stimulates, induces, promotes, increases, or enhances an antibody response against two or more different serotypes of a flavivirus and two or more different species of flavivirus and a T cell response against two or more different serotypes of a flavivirus and two or more different species of flavivirus.

3. The composition of claim 1, wherein the protein or peptide, or variant, homologue, derivative, or subsequence thereof, that comprises, consists, or consists essentially of a flavivirus B cell epitope, is a flavivirus envelope, membrane, premembrane, core, NS1, NS2A, NS2B, NS3, NS4A, NS4B or NS5 protein or peptide or wherein the protein or peptide, or variant, homologue, derivative, or subsequence thereof, that comprises, consists, or consists essentially of a flavivirus T cell epitope, is a flavivirus envelope, membrane, premembrane, core, NS1, NS2A, NS2B, NS3, NS4A, NS4B or NS5 protein or peptide.

4.-8. (canceled)

9. The composition of claim 1, comprising proteins or peptides, or variants, homologues, derivatives, or subsequences thereof from DENV 1-4 Dengue virus serotypes or Zika virus, or nucleic acid molecules encoding the proteins or peptides, or variants, homologues, derivatives, or subsequences thereof, from DENV 1-4 Dengue virus serotypes or Zika virus.

10.-14. (canceled)

15. The composition of claim 1, comprising a protein, or variant, homologue, derivative or subsequence thereof, that comprises, consists or consists essentially of a consensus amino acid sequence derived from proteins or peptides from two or more different serotypes of a flavivirus or proteins or peptides from two or more different species of flavivirus, or nucleic acid molecules encoding the consensus amino acid sequence derived from proteins or peptides from two or more different serotypes of a flavivirus or proteins or peptides from two or more different species of flavivirus.

16.-21. (canceled)

22. The composition of claim 1, further comprising a CD70 protein or peptide, or variant, homologue, derivative, or subsequence thereof, or a nucleic acid molecule encoding a CD70 protein, or variant, homologue, derivative, or subsequence thereof.

23.-27. (canceled)

28. A method of eliciting, stimulating, inducing, promoting, increasing, or enhancing an immune response against a flavivirus, the method comprising administering the composition of claim 1.

29. The method of claim 28, wherein the composition elicits, stimulates, induces, promotes, increases, or enhances an immune response against two or more different serotypes of a flavivirus or two or more different species of flavivirus.

30.-36. (canceled)

37. A composition comprising (a) at least one polypeptide having an amino acid sequence at least 90% identical to a sequence set forth as SEQ ID NO: 11-14, (b) at least one polypeptide having an amino acid sequence at least 90% identical to a sequence set forth as SEQ ID NO: 1-5, at least one polypeptide having an amino acid sequence at least 90% identical to a sequence set forth as SEQ ID NO: 6-10, at least one polypeptide having an amino acid sequence at least 90% identical to a sequence set forth as SEQ ID NO: 15-19, or at least one polypeptide having an amino acid sequence at least 90% identical to a sequence set forth as SEQ ID NO: 20-24, and (c) a pharmaceutically acceptable buffer or excipient.

38. The composition of claim 37, comprising at least two polypeptides having an amino acid sequence at least 90% identical to a sequence set forth as SEQ ID NO: 1-5, at least two polypeptides having an amino acid sequence at least 90% identical to a sequence set forth as SEQ ID NO: 6-10, at least two polypeptide having an amino acid sequence at least 90% identical to a sequence set forth as SEQ ID NO: 15-19, or at least two polypeptide having an amino acid sequence at least 90% identical to a sequence set forth as SEQ ID NO: 20-24.

39.-48. (canceled)

49. The composition of claim 37, further comprising an effective amount of an adjuvant.

50. (canceled)

51. The composition of claim 49, wherein the adjuvant comprises CD70.

52. The composition of claim 51, wherein the adjuvant comprises a nucleic acid encoding a CD70 polypeptide.

53.-54. (canceled)

55. A composition comprising (a) at least one polynucleotide encoding an amino acid sequence at least 90% identical to a sequence set forth as SEQ ID NO: 11-14, (b) at least one polynucleotide encoding an amino acid sequence at least 90% identical to a sequence set forth as SEQ ID NO: 1-5, at least one polynucleotide encoding an amino acid sequence at least 90% identical to a sequence set forth as SEQ ID NO: 6-10, at least one polynucleotide encoding an amino acid sequence at least 90% identical to a sequence set forth as SEQ ID NO: 15-19, or at least one polynucleotide encoding an amino acid sequence at least 90% identical to a sequence set forth as SEQ ID NO: 20-24, and (c) a pharmaceutically acceptable buffer or excipient.

56. The composition of claim 55, comprising at least two polynucleotides encoding an amino acid sequence at least 90% identical to a sequence set forth as SEQ ID NO: 1-5, at least two polynucleotide encoding an amino acid sequence at least 90% identical to a sequence set forth as SEQ ID NO: 6-10, at least two polynucleotide encoding an amino acid sequence at least 90% identical to a sequence set forth as SEQ ID NO: 15-19, or at least two polynucleotide encoding an amino acid sequence at least 90% identical to a sequence set forth as SEQ ID NO: 20-24.

57.-66. (canceled)

67. The composition of claim 55, further comprising an effective amount of an adjuvant.

68. (canceled)

69. The composition of claim 67, wherein the adjuvant comprises CD70.

70. The composition of claim 69, wherein the adjuvant comprises a nucleic acid encoding a CD70 polypeptide.

71.-106. (canceled)

107. A method of eliciting an immune response against a flavivirus in a subject, the method comprising administering an effective amount of the composition of claim 37.

108. The method of claim 107, wherein the flavivirus comprises a dengue virus or a zika virus.

109.-117. (canceled)
